SEQUENCE LISTING (327 sequences)

SEQ ID NO: 1 - protein, 126 aa, synthetic construct
  source 1..126: note = Synthetic Construct
EVQLVESGGGLVQPGGSLRLSCAASGRAFSDYAMAWFRQAPGQEREFVAGIGWSGGDTLYADSVRGRFTNSRDNSKNTLYLQMNSLRAEDTAVYYCAARQGQYIYSSMRSDSYDYWGQGTLVTVSS

SEQ ID NO: 2 - protein, 126 aa, synthetic construct
  source 1..126: note = Synthetic Construct
EVQLVESGGGLVQPGGSLRLSCAASGRAFSDYAMAWFRQAPGQEREFVAGIGWSGGDTLYADSVRGRFTISRDNAKNTLYLQMNSLRAEDTAVYYCAARQGQYIYSSMRSDSYDYWGQGTLVTVSS

SEQ ID NO: 3 - protein, 126 aa, synthetic construct
  source 1..126: note = Synthetic Construct
EVQLVESGGGLVQPGGSLRLSCAASGRAFSDYAMAWFRQAPGQEREFVAGIGWSGGDTLYADSVRGRFTISRDNSKNTMYLQMNSLRAEDTAVYYCAARQGQYIYSSMRSDSYDYWGQGTLVTVSS

SEQ ID NO: 4 - protein, 126 aa, synthetic construct
  source 1..126: note = Synthetic Construct
EVQLVESGGGLVQPGGSLRLSCAASGRAFSDYAMAWFRQAPGQGLEFVAGIGWSGGDTLYADSVRGRFTISRDNSKNTLYLQMNSLRAEDTAVYYCAARQGQYIYSSMRSDSYDYWGQGTLVTVSS

SEQ ID NO: 5 - protein, 132 aa, synthetic construct
  source 1..132: note = Synthetic Construct
EVQLVESGGGLVQPGGSLRLSCAASGRTFSGILSPYAVGWFRQAPGKGLEFVSTITSGGSAIYTDSVKGRFTISRDNAKDSLYLQMNSLRAEDTAVYYCAVRTRRYGSNLGEVPQENEYGYWGQGTLVTVSS

SEQ ID NO: 6 - protein, 132 aa, synthetic construct
  source 1..132: note = Synthetic Construct
EVQLVESGGGLVQPGGSLRLSCAASGRTFSGILSPYAVGWFRQAPGKGLEFVSTITSGGSAIYTDSVKGRFTISRDNAKNTLYLQMNSLRAEDTAVYYCAVRTRRYGSNLGEVPQENEYGYWGQGTLVTVSS

SEQ ID NO: 7 - protein, 132 aa, synthetic construct
  source 1..132: note = Synthetic Construct
EVQLVESGGGLVQPGGSLRLSCAASGRTFSGILSPYAVGWFRQAPGKGLEFVSTITSGGSAIYTDSVKGRFTISRDNAKNSVYLQMNSLRAEDTAVYYCAVRTRRYGSNLGEVPQENEYGYWGQGTLVTVSS

SEQ ID NO: 8 - protein, 132 aa, synthetic construct
  source 1..132: note = Synthetic Construct
EVQLVESGGGLVQPGGSLRLSCAASGRTFSGILSPYAVGWFRQAPGQGLEFVATITSGGSAIYTDSVKGRFTISRDNSKNTLYLQMNSLRAEDTAVYYCAVRTRRYGSNLGEVPQENEYGYWGQGTLVTVSS

SEQ ID NO: 9 - protein, 126 aa, synthetic construct
  source 1..126: note = Synthetic Construct
EVQLVESGGGLVQPGGSLRLSCAASGRHFSDYAMAWFRQAPGQEREFVAGIGWSGGDTLYADSVRGRFTISRDNSKNTLYLQMNSLRAEDTAVYYCAARQGQYIYSSMRSDSYDYWGQGTLVTVSS

SEQ ID NO: 10 - protein, 126 aa, synthetic construct
  source 1..126: note = Synthetic Construct
EVQLVESGGGLVQPGGSLRLSCAASGRAHSDYAMAWFRQAPGQEREFVAGIGWSGGDTLYADSVRGRFTISRDNSKNTLYLQMNSLRAEDTAVYYCAARQGQYIYSSMRSDSYDYWGQGTLVTVSS

SEQ ID NO: 11 - protein, 126 aa, synthetic construct
  source 1..126: note = Synthetic Construct
EVQLVESGGGLVQPGGSLRLSCAASGRAHSDYAMAWFRQAPGQEREFVAGIGWSGGDTLYADSVRGRFTNSRDNSKNTLYLQMNSLRAEDTAVYYCAARQGQYIYSSMRSDSYDYWGQGTLVTVSS

SEQ ID NO: 12 - protein, 126 aa, synthetic construct
  source 1..126: note = Synthetic Construct
EVQLVESGGGLVQPGGSLRLSCAASGRHHSDYAMAWFRQAPGQEREFVAGIGWSGGDTLYADSVRGRFTISRDNSKNTLYLQMNSLRAEDTAVYYCAARQGQYIYSSMRSDSYDYWGQGTLVTVSS

SEQ ID NO: 13 - protein, 10 aa, synthetic construct
  source 1..10: note = Synthetic Construct
GRAFSDYAMA

SEQ ID NO: 14 - protein, 14 aa, synthetic construct
  source 1..14: note = Synthetic Construct
GRTFSGILSPYAVG

SEQ ID NO: 15 - protein, 10 aa, synthetic construct
  source 1..10: note = Synthetic Construct
GRHFSDYAMA

SEQ ID NO: 16 - protein, 10 aa, synthetic construct
  source 1..10: note = Synthetic Construct
GRAHSDYAMA

SEQ ID NO: 17 - protein, 10 aa, synthetic construct
  source 1..10: note = Synthetic Construct
GRHHSDYAMA

SEQ ID NO: 18 - protein, 17 aa, synthetic construct
  source 1..17: note = Synthetic Construct
GIGWSGGDTLYADSVRG

SEQ ID NO: 19 - protein, 16 aa, synthetic construct
  source 1..16: note = Synthetic Construct
TITSGGSAIYTDSVKG

SEQ ID NO: 20 - protein, 19 aa, synthetic construct
  source 1..19: note = Synthetic Construct
AARQGQYIYSSMRSDSYDY

SEQ ID NO: 21 - protein, 22 aa, synthetic construct
  source 1..22: note = Synthetic Construct
AVRTRRYGSNLGEVPQENEYGY

SEQ ID NO: 22 - protein, 127 aa, synthetic construct
  source 1..127: note = Synthetic Construct
QVQLVESGGGLVQAGGSLRLSCAASGRTFGSDAAGWFRQASGKEREFVASISWSGGYTYYADSVKGRFTISSDNVKNTVYLQMNSLTPEDTAVYFCATGNRYSDYRISLVTPSQYEYWGQGTLVTVS

SEQ ID NO: 23 - protein, 124 aa, synthetic construct
  source 1..124: note = Synthetic Construct
QVQLVESGGGLVQPGGSLRLSCTGSGHSFSTYTVGWFRQAPGEERKFVASISWSGEVTLYGDSVKGRFTISRDNRKKTVYLQMHSLKPEDSAIYYCAAKRGGRPTDSSDDYFYWGQGTQVTVSS

SEQ ID NO: 24 - protein, 123 aa, synthetic construct
  source 1..123: note = Synthetic Construct
QVQLNESGGGMVQAGGSLRLSCAASGRTVSNYAAGWFRQAPGKEREFVAAINWNKTTTYADSVKGRFIISREYAKNTVALQMNSLKPEDTAVYYCAAVFRIVAPKTQYEYDYWGQGTQVTVSS

SEQ ID NO: 25 - protein, 123 aa, synthetic construct
  source 1..123: note = Synthetic Construct
QVQLIESGGGLVQAGGSLGLSCAASGRPVSNYAAAWFRQAPGKEREFVAAINWNKTATYADSVKGRFTISRDNAKSTVALQMNSLKPEDTAVYYCAAVFRVVAPKTQYDYDYWGQGTQVTVSS

SEQ ID NO: 26 - protein, 123 aa, synthetic construct
  source 1..123: note = Synthetic Construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSS

SEQ ID NO: 27 - protein, 123 aa, synthetic construct
  source 1..123: note = Synthetic Construct
QVQLVESGGGLVQAGGSLRLSCAASGRTFSSYAIGWFRQAPGKAREFVARVSTIAGDTDYADSVKGRFTISRDNAKNTVYLQMNSLKPEDTAVYYCAADSYNVRLVTGEADYWGEGTQVTVSS

SEQ ID NO: 28 - protein, 123 aa, synthetic construct
  source 1..123: note = Synthetic Construct
QVQLVESGGGLVQAGGSLRLSCAASGRTFSSYAIGWFRQAPGKAREFVARVSTIAGDTDYADSVKGRFTISRDNAKNTVYLQMNSLKPEDTAVYYCAADSYNVRLGTGEADYWGEGTQVTVSS

SEQ ID NO: 29 - protein, 123 aa, synthetic construct
  source 1..123: note = Synthetic Construct
EVQLVESGGGLVQAGDSLRLSCAASGFTFSNYAIGWFRQAPGKAREFVARVSTIAGDTDYANAVKGRFTISRDNAKNTVYLQMNSLKPDDTAVYYCAAESYNVRLVTGEADYWGEGTQVTVSS

SEQ ID NO: 30 - protein, 127 aa, synthetic construct
  source 1..127: note = Synthetic Construct
QVRLAESGGGRVQAGESLRLSCVASGRTFSNDAAGWFREASGKEREFVASISWSGNYTYYADSVKGRFTISEDNVKNTVYLQMTSLKPEDTAVYYCAAGNRYSDYRISLVTPRLYEYWGQGTQVTVS

SEQ ID NO: 31 - protein, 127 aa, synthetic construct
  source 1..127: note = Synthetic Construct
QVQLVESGGGLVQAGGSLRLSCAASGRTFSSDAAGWFRQASGKEREFVAAISWSGNYTYSADSVKGRFTISSDNVKNTVYLQMNSLKPEDTAVYLCAAGNRYSDYRISLVTPSQYEYWGQGTQVTVS

SEQ ID NO: 32 - protein, 127 aa, synthetic construct
  source 1..127: note = Synthetic Construct
QVQLVESGGGLVQAGGSLRLSCAASGRTFGSDAAGWFRQASGKEREFVASISWSGGYTYYADSGTGRFTISSDNVKNTVYLQMNSLTPEDTAVYFCATGNRDSDYRISLVTPSQYEYWGQGTQVTVS

SEQ ID NO: 33 - protein, 127 aa, synthetic construct
  source 1..127: note = Synthetic Construct
QVQLVESGGGLVQAGGSLRLSCAASGRTFGSDAAGWFRQASGKEREFVASISWSGGYTYYADSGKGRFTISSDNVKNTVYLQMNSLTPEDTAVYFCATGNRYSDYRISLVTPSQYDYWGQGTQVTVS

SEQ ID NO: 34 - protein, 127 aa, synthetic construct
  source 1..127: note = Synthetic Construct
QVQLVESGGGLVQAGGSLRLSCAASGRTFGSDAAGWFRQASGKEREFVASISWSGGYTYYADSVKGRFTSSSDNVKNTVYLQMNSLTPEDTAVYFCATVNRYSDYRISLVTPSQYEYWGQGTQVTVS

SEQ ID NO: 35 - protein, 8 aa, synthetic construct
  source 1..8: note = Synthetic Construct
GRTFGSDA

SEQ ID NO: 36 - protein, 8 aa, synthetic construct
  source 1..8: note = Synthetic Construct
GHSFSTYT

SEQ ID NO: 37 - protein, 8 aa, synthetic construct
  source 1..8: note = Synthetic Construct
GRTVSNYA

SEQ ID NO: 38 - protein, 8 aa, synthetic construct
  source 1..8: note = Synthetic Construct
GRPVSNYA

SEQ ID NO: 39 - protein, 8 aa, synthetic construct
  source 1..8: note = Synthetic Construct
GRTFSSYA

SEQ ID NO: 40 - protein, 8 aa, synthetic construct
  source 1..8: note = Synthetic Construct
GFTFSNYA

SEQ ID NO: 41 - protein, 8 aa, synthetic construct
  source 1..8: note = Synthetic Construct
GRTFSNDA

SEQ ID NO: 42 - protein, 8 aa, synthetic construct
  source 1..8: note = Synthetic Construct
GRTFSSDA

SEQ ID NO: 43 - protein, 8 aa, synthetic construct
  source 1..8: note = Synthetic Construct
GRTFGSDA

SEQ ID NO: 44 - protein, 8 aa, synthetic construct
  source 1..8: note = Synthetic Construct
ISWSGGYT

SEQ ID NO: 45 - protein, 8 aa, synthetic construct
  source 1..8: note = Synthetic Construct
ISWSGEVT

SEQ ID NO: 46 - protein, 8 aa, synthetic construct
  source 1..8: note = Synthetic Construct
INWNKTTT

SEQ ID NO: 47 - protein, 8 aa, synthetic construct
  source 1..8: note = Synthetic Construct
INWNKTAT

SEQ ID NO: 48 - protein, 8 aa, synthetic construct
  source 1..8: note = Synthetic Construct
INWQKTAT

SEQ ID NO: 49 - protein, 8 aa, synthetic construct
  source 1..8: note = Synthetic Construct
VSTIAGDT

SEQ ID NO: 50 - protein, 8 aa, synthetic construct
  source 1..8: note = Synthetic Construct
ISWSGNYT

SEQ ID NO: 51 - protein, 8 aa, synthetic construct
  source 1..8: note = Synthetic Construct
ISWSGGYT

SEQ ID NO: 52 - protein, 21 aa, synthetic construct
  source 1..21: note = Synthetic Construct
ATGNRYSDYRISLVTPSQYEY

SEQ ID NO: 53 - protein, 17 aa, synthetic construct
  source 1..17: note = Synthetic Construct
AAKRGGRPTDSSDDYFY

SEQ ID NO: 54 - protein, 17 aa, synthetic construct
  source 1..17: note = Synthetic Construct
AAVFRIVAPKTQYEYDY

SEQ ID NO: 55 - protein, 17 aa, synthetic construct
  source 1..17: note = Synthetic Construct
AAVFRVVAPKTQYDYDY

SEQ ID NO: 56 - protein, 16 aa, synthetic construct
  source 1..16: note = Synthetic Construct
AADSYNVRLVTGEADY

SEQ ID NO: 57 - protein, 16 aa, synthetic construct
  source 1..16: note = Synthetic Construct
AADSYNVRLGTGEADY

SEQ ID NO: 58 - protein, 16 aa, synthetic construct
  source 1..16: note = Synthetic Construct
AAESYNVRLVTGEADY

SEQ ID NO: 59 - protein, 21 aa, synthetic construct
  source 1..21: note = Synthetic Construct
AAGNRYSDYRISLVTPRLYEY

SEQ ID NO: 60 - protein, 21 aa, synthetic construct
  source 1..21: note = Synthetic Construct
AAGNRYSDYRISLVTPSQYEY

SEQ ID NO: 61 - protein, 21 aa, synthetic construct
  source 1..21: note = Synthetic Construct
ATGNRDSDYRISLVTPSQYEY

SEQ ID NO: 62 - protein, 21 aa, synthetic construct
  source 1..21: note = Synthetic Construct
ATGNRYSDYRISLVTPSQYDY

SEQ ID NO: 63 - protein, 21 aa, synthetic construct
  source 1..21: note = Synthetic Construct
ATVNRYSDYRISLVTPSQYEY

SEQ ID NO: 64 - protein, 256 aa, synthetic construct
  source 1..256: note = Synthetic Construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSGGGGSGGGGSEVQLVESGGGLVQPGGSLRLSCAASGRHFSDYAMAWFRQAPGQEREFVAGIGWSGGDTLYADSVRGRFTISRDNAKNTLYLQMNSLRAEDTAVYYCAARQGQYIYSSMRSDSYDYWGQGTLVTVSS

SEQ ID NO: 65 - protein, 256 aa, synthetic construct
  source 1..256: note = Synthetic Construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSGGGGSGGGGSEVQLVESGGGLVQPGGSLRLSCAASGRAHSDYAMAWFRQAPGQEREFVAGIGWSGGDTLYADSVRGRFTISRDNAKNTLYLQMNSLRAEDTAVYYCAARQGQYIYSSMRSDSYDYWGQGTLVTVSS

SEQ ID NO: 66 - protein, 256 aa, synthetic construct
  source 1..256: note = Synthetic Construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSGGGGSGGGGSEVQLVESGGGLVQPGGSLRLSCAASGRHHSDYAMAWFRQAPGQEREFVAGIGWSGGDTLYADSVRGRFTISRDNAKNTLYLQMNSLRAEDTAVYYCAARQGQYIYSSMRSDSYDYWGQGTLVTVSS

SEQ ID NO: 67 - protein, 256 aa, synthetic construct
  source 1..256: note = Synthetic Construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSGGGGSGGGGSEVQLVESGGGLVQPGGSLRLSCAASGRHFSDYAMAWFRQAPGQEREFVAGIGWSGGDTLYADSVRGRFTISRDNSKNTMYLQMNSLRAEDTAVYYCAARQGQYIYSSMRSDSYDYWGQGTLVTVSS

SEQ ID NO: 68 - protein, 256 aa, synthetic construct
  source 1..256: note = Synthetic Construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSGGGGSGGGGSEVQLVESGGGLVQPGGSLRLSCAASGRAHSDYAMAWFRQAPGQEREFVAGIGWSGGDTLYADSVRGRFTISRDNSKNTMYLQMNSLRAEDTAVYYCAARQGQYIYSSMRSDSYDYWGQGTLVTVSS

SEQ ID NO: 69 - protein, 256 aa, synthetic construct
  source 1..256: note = Synthetic Construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSGGGGSGGGGSEVQLVESGGGLVQPGGSLRLSCAASGRHHSDYAMAWFRQAPGQEREFVAGIGWSGGDTLYADSVRGRFTISRDNSKNTMYLQMNSLRAEDTAVYYCAARQGQYIYSSMRSDSYDYWGQGTLVTVSS

SEQ ID NO: 70 - protein, 264 aa, synthetic construct
  source 1..264: note = Synthetic Construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGSGGGGSGGGGSEVQLVESGGGLVQPGGSLRLSCAASGRHFSDYAMAWFRQAPGQEREFVAGIGWSGGDTLYADSVRGRFTISRDNAKNTLYLQMNSLRAEDTAVYYCAARQGQYIYSSMRSDSYDYWGQGTLVTVSS

SEQ ID NO: 71 - protein, 264 aa, synthetic construct
  source 1..264: note = Synthetic Construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGSGGGGSGGGGSEVQLVESGGGLVQPGGSLRLSCAASGRAHSDYAMAWFRQAPGQEREFVAGIGWSGGDTLYADSVRGRFTISRDNAKNTLYLQMNSLRAEDTAVYYCAARQGQYIYSSMRSDSYDYWGQGTLVTVSS

SEQ ID NO: 72 - protein, 264 aa, synthetic construct
  source 1..264: note = Synthetic Construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGSGGGGSGGGGSEVQLVESGGGLVQPGGSLRLSCAASGRHHSDYAMAWFRQAPGQEREFVAGIGWSGGDTLYADSVRGRFTISRDNAKNTLYLQMNSLRAEDTAVYYCAARQGQYIYSSMRSDSYDYWGQGTLVTVSS

SEQ ID NO: 73 - protein, 264 aa, synthetic construct
  source 1..264: note = Synthetic Construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGSGGGGSGGGGSEVQLVESGGGLVQPGGSLRLSCAASGRHFSDYAMAWFRQAPGQEREFVAGIGWSGGDTLYADSVRGRFTISRDNSKNTMYLQMNSLRAEDTAVYYCAARQGQYIYSSMRSDSYDYWGQGTLVTVSS

SEQ ID NO: 74 - protein, 264 aa, synthetic construct
  source 1..264: note = Synthetic Construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGSGGGGSGGGGSEVQLVESGGGLVQPGGSLRLSCAASGRAHSDYAMAWFRQAPGQEREFVAGIGWSGGDTLYADSVRGRFTISRDNSKNTMYLQMNSLRAEDTAVYYCAARQGQYIYSSMRSDSYDYWGQGTLVTVSS

SEQ ID NO: 75 - protein, 264 aa, synthetic construct
  source 1..264: note = Synthetic Construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGSGGGGSGGGGSEVQLVESGGGLVQPGGSLRLSCAASGRHHSDYAMAWFRQAPGQEREFVAGIGWSGGDTLYADSVRGRFTISRDNSKNTMYLQMNSLRAEDTAVYYCAARQGQYIYSSMRSDSYDYWGQGTLVTVSS

SEQ ID NO: 76 - protein, 262 aa, synthetic construct
  source 1..262: note = Synthetic Construct
EVQLVESGGGLVQPGGSLRLSCAASGRTFSGILSPYAVGWFRQAPGKGLEFVSTITSGGSAIYTDSVKGRFTISRDNAKDSLYLQMNSLRAEDTAVYYCAVRTRRYGSNLGEVPQENEYGYWGQGTLVTVSSGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 77 - protein, 270 aa, synthetic construct
  source 1..270: note = Synthetic Construct
EVQLVESGGGLVQPGGSLRLSCAASGRTFSGILSPYAVGWFRQAPGKGLEFVSTITSGGSAIYTDSVKGRFTISRDNAKDSLYLQMNSLRAEDTAVYYCAVRTRRYGSNLGEVPQENEYGYWGQGTLVTVSSGGGGSGGGGSGGGGSEVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSS

SEQ ID NO: 78 - protein, 262 aa, synthetic construct
  source 1..262: note = Synthetic Construct
EVQLVESGGGLVQPGGSLRLSCAASGRTFSGILSPYAVGWFRQAPGKGLEFVSTITSGGSAIYTDSVKGRFTISRDNAKNTLYLQMNSLRAEDTAVYYCAVRTRRYGSNLGEVPQENEYGYWGQGTLVTVSSGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 79 - protein, 270 aa, synthetic construct
  source 1..270: note = Synthetic Construct
EVQLVESGGGLVQPGGSLRLSCAASGRTFSGILSPYAVGWFRQAPGKGLEFVSTITSGGSAIYTDSVKGRFTISRDNAKNTLYLQMNSLRAEDTAVYYCAVRTRRYGSNLGEVPQENEYGYWGQGTLVTVSSGGGGSGGGGSGGGGSEVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSS

SEQ ID NO: 80 - protein, 262 aa, synthetic construct
  source 1..262: note = Synthetic Construct
EVQLVESGGGLVQPGGSLRLSCAASGRTFSGILSPYAVGWFRQAPGKGLEFVSTITSGGSAIYTDSVKGRFTISRDNAKNSVYLQMNSLRAEDTAVYYCAVRTRRYGSNLGEVPQENEYGYWGQGTLVTVSSGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 81 - protein, 270 aa, synthetic construct
  source 1..270: note = Synthetic Construct
EVQLVESGGGLVQPGGSLRLSCAASGRTFSGILSPYAVGWFRQAPGKGLEFVSTITSGGSAIYTDSVKGRFTISRDNAKNSVYLQMNSLRAEDTAVYYCAVRTRRYGSNLGEVPQENEYGYWGQGTLVTVSSGGGGSGGGGSGGGGSEVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSS

SEQ ID NO: 82 - protein, 262 aa, synthetic construct
  source 1..262: note = Synthetic Construct
EVQLVESGGGLVQPGGSLRLSCAASGRTFSGILSPYAVGWFRQAPGQGLEFVATITSGGSAIYTDSVKGRFTISRDNSKNTLYLQMNSLRAEDTAVYYCAVRTRRYGSNLGEVPQENEYGYWGQGTLVTVSSGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 83 - protein, 270 aa, synthetic construct
  source 1..270: note = Synthetic Construct
EVQLVESGGGLVQPGGSLRLSCAASGRTFSGILSPYAVGWFRQAPGQGLEFVATITSGGSAIYTDSVKGRFTISRDNSKNTLYLQMNSLRAEDTAVYYCAVRTRRYGSNLGEVPQENEYGYWGQGTLVTVSSGGGGSGGGGSGGGGSEVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSS

SEQ ID NO: 84 - protein, 256 aa, synthetic construct
  source 1..256: note = Synthetic Construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSGGGGSGGGGSEVQLVESGGGLVQPGGSLRLSCAASGRAFSDYAMAWFRQAPGQEREFVAGIGWSGGDTLYADSVRGRFTNSRDNSKNTLYLQMNSLRAEDTAVYYCAARQGQYIYSSMRSDSYDYWGQGTLVTVSS

SEQ ID NO: 85 - protein, 264 aa, synthetic construct
  source 1..264: note = Synthetic Construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGSGGGGSGGGGSEVQLVESGGGLVQPGGSLRLSCAASGRAFSDYAMAWFRQAPGQEREFVAGIGWSGGDTLYADSVRGRFTNSRDNSKNTLYLQMNSLRAEDTAVYYCAARQGQYIYSSMRSDSYDYWGQGTLVTVSS

SEQ ID NO: 86 - protein, 256 aa, synthetic construct
  source 1..256: note = Synthetic Construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSGGGGSGGGGSEVQLVESGGGLVQPGGSLRLSCAASGRAFSDYAMAWFRQAPGQEREFVAGIGWSGGDTLYADSVRGRFTISRDNAKNTLYLQMNSLRAEDTAVYYCAARQGQYIYSSMRSDSYDYWGQGTLVTVSS

SEQ ID NO: 87 - protein, 264 aa, synthetic construct
  source 1..264: note = Synthetic Construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGSGGGGSGGGGSEVQLVESGGGLVQPGGSLRLSCAASGRAFSDYAMAWFRQAPGQEREFVAGIGWSGGDTLYADSVRGRFTISRDNAKNTLYLQMNSLRAEDTAVYYCAARQGQYIYSSMRSDSYDYWGQGTLVTVSS

SEQ ID NO: 88 - protein, 256 aa, synthetic construct
  source 1..256: note = Synthetic Construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSGGGGSGGGGSEVQLVESGGGLVQPGGSLRLSCAASGRAFSDYAMAWFRQAPGQEREFVAGIGWSGGDTLYADSVRGRFTISRDNSKNTMYLQMNSLRAEDTAVYYCAARQGQYIYSSMRSDSYDYWGQGTLVTVSS

SEQ ID NO: 89 - protein, 264 aa, synthetic construct
  source 1..264: note = Synthetic Construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGSGGGGSGGGGSEVQLVESGGGLVQPGGSLRLSCAASGRAFSDYAMAWFRQAPGQEREFVAGIGWSGGDTLYADSVRGRFTISRDNSKNTMYLQMNSLRAEDTAVYYCAARQGQYIYSSMRSDSYDYWGQGTLVTVSS

SEQ ID NO: 90 - protein, 256 aa, synthetic construct
  source 1..256: note = Synthetic Construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSGGGGSGGGGSEVQLVESGGGLVQPGGSLRLSCAASGRAFSDYAMAWFRQAPGQGLEFVAGIGWSGGDTLYADSVRGRFTISRDNSKNTLYLQMNSLRAEDTAVYYCAARQGQYIYSSMRSDSYDYWGQGTLVTVSS

SEQ ID NO: 91 - protein, 264 aa, synthetic construct
  source 1..264: note = Synthetic Construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGSGGGGSGGGGSEVQLVESGGGLVQPGGSLRLSCAASGRAFSDYAMAWFRQAPGQGLEFVAGIGWSGGDTLYADSVRGRFTISRDNSKNTLYLQMNSLRAEDTAVYYCAARQGQYIYSSMRSDSYDYWGQGTLVTVSS

SEQ ID NO: 92 - protein, 256 aa, synthetic construct
  source 1..256: note = Synthetic Construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSGGGGSGGGGSEVQLVESGGGLVQPGGSLRLSCAASGRAHSDYAMAWFRQAPGQEREFVAGIGWSGGDTLYADSVRGRFTNSRDNSKNTLYLQMNSLRAEDTAVYYCAARQGQYIYSSMRSDSYDYWGQGTLVTVSS

SEQ ID NO: 93 - protein, 264 aa, synthetic construct
  source 1..264: note = Synthetic Construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGSGGGGSGGGGSEVQLVESGGGLVQPGGSLRLSCAASGRAHSDYAMAWFRQAPGQEREFVAGIGWSGGDTLYADSVRGRFTNSRDNSKNTLYLQMNSLRAEDTAVYYCAARQGQYIYSSMRSDSYDYWGQGTLVTVSS

SEQ ID NO: 94 - protein, 256 aa, synthetic construct
  source 1..256: note = Synthetic Construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSGGGGSGGGGSEVQLVESGGGLVQPGGSLRLSCAASGRAHSDYAMAWFRQAPGQGLEFVAGIGWSGGDTLYADSVRGRFTISRDNSKNTLYLQMNSLRAEDTAVYYCAARQGQYIYSSMRSDSYDYWGQGTLVTVSS

SEQ ID NO: 95 - protein, 264 aa, synthetic construct
  source 1..264: note = Synthetic Construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGSGGGGSGGGGSEVQLVESGGGLVQPGGSLRLSCAASGRAHSDYAMAWFRQAPGQGLEFVAGIGWSGGDTLYADSVRGRFTISRDNSKNTLYLQMNSLRAEDTAVYYCAARQGQYIYSSMRSDSYDYWGQGTLVTVSS

SEQ ID NO: 96 - protein, 264 aa, synthetic construct
  source 1..264: note = Synthetic Construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGAGGGGAGGGGSEVQLVESGGGLVQPGGSLRLSCAASGRAHSDYAMAWFRQAPGQEREFVAGIGWSGGDTLYADSVRGRFTNSRDNSKNTLYLQMNSLRAEDTAVYYCAARQGQYIYSSMRSDSYDYWGQGTLVTVSS

SEQ ID NO: 97 - protein, 270 aa, synthetic construct
  source 1..270: note = Synthetic Construct
EVQLVESGGGLVQPGGSLRLSCAASGRTFSGILSPYAVGWFRQAPGKGLEFVSTITSGGSAIYTDSVKGRFTISRDNAKDSLYLQMNSLRAEDTAVYYCAVRTRRYGSNLGEVPQENEYGYWGQGTLVTVSSGGGGAGGGGAGGGGSEVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSS

SEQ ID NO: 98 - protein, 266 aa, synthetic construct
  source 1..266: note = Synthetic Construct
EVQLVESGGGVVQAGDSLTLTCTAPVGTISDYGMGWFRQAPGKEREFVASISWGGMWTDYADSVKGRFTISRDNDKNAVYLRMNSLNAEDTAVYYCGRGRMYRGIGNSLAQPKSYGYWGQGTQVTVSSGGGGAGGGGAGGGGSEVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSS

SEQ ID NO: 99 - protein, 270 aa, synthetic construct
  source 1..270: note = Synthetic Construct
EVQLVESGGGLVQAGGSLRLSCAASGRTFSGILSAYAVGWFRQAPGKEREFVSTITSGGSTLSADSVKGRFTLSRDNAKDTVYLQMNSLKPEDTAVYYCAVRTWPYGSNRGEVPTENEYGHWGQGTQVTVSSGGGGAGGGGAGGGGSEVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSS

SEQ ID NO: 100 - protein, 266 aa, synthetic construct
  source 1..266: note = Synthetic Construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGAGGGGAGGGGSEVQLVESGGGVVQAGDSLTLTCTAPVGTISDYGMGWFRQAPGKEREFVASISWGGMWTDYADSVKGRFTISRDNDKNAVYLRMNSLNAEDTAVYYCGRGRMYRGIGNSLAQPKSYGYWGQGTQVTVSS

SEQ ID NO: 101 - protein, 270 aa, synthetic construct
  source 1..270: note = Synthetic Construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGAGGGGAGGGGSEVQLVESGGGLVQAGGSLRLSCAASGRTFSGILSAYAVGWFRQAPGKEREFVSTITSGGSTLSADSVKGRFTLSRDNAKDTVYLQMNSLKPEDTAVYYCAVRTWPYGSNRGEVPTENEYGHWGQGTQVTVSS

SEQ ID NO: 102 - protein, 15 aa, synthetic construct
  source 1..15: note = Synthetic Construct
GGGGAGGGGAGGGGS

SEQ ID NO: 103 - protein, 15 aa, synthetic construct
  source 1..15: note = Synthetic Construct
GGGGSGGGGSGGGGS

SEQ ID NO: 104 - protein, 5 aa, synthetic construct
  source 1..5: note = Synthetic Construct
GGGGS

SEQ ID NO: 105 - protein, 10 aa, synthetic construct
  source 1..10: note = Synthetic Construct
GGGGSGGGGS

SEQ ID NO: 106 - protein, 15 aa, synthetic construct
  source 1..15: note = Synthetic Construct
GGGGSGGGGSGGGGS

SEQ ID NO: 107 - protein, 20 aa, synthetic construct
  source 1..20: note = Synthetic Construct
GGGGSGGGGSGGGGSGGGGS

SEQ ID NO: 108 - protein, 25 aa, synthetic construct
  source 1..25: note = Synthetic Construct
GGGGSGGGGSGGGGSGGGGSGGGGS

SEQ ID NO: 109 - protein, 30 aa, synthetic construct
  source 1..30: note = Synthetic Construct
GGGGSGGGGSGGGGSGGGGSGGGGSGGGGS

SEQ ID NO: 110 - protein, 15 aa, synthetic construct
  source 1..15: note = Synthetic Construct
EAAAKEAAAKEAAAK

SEQ ID NO: 111 - protein, 5 aa, synthetic construct
  source 1..5: note = Synthetic Construct
PAPAP

SEQ ID NO: 112 - protein, 10 aa, synthetic construct
  source 1..10: note = Synthetic Construct
GGGGSPAPAP

SEQ ID NO: 113 - protein, 10 aa, synthetic construct
  source 1..10: note = Synthetic Construct
PAPAPGGGGS

SEQ ID NO: 114 - protein, 12 aa, synthetic construct
  source 1..12: note = Synthetic Construct
GSTSGKSSEGKG

SEQ ID NO: 115 - protein, 10 aa, synthetic construct
  source 1..10: note = Synthetic Construct
GGGDSGGGDS

SEQ ID NO: 116 - protein, 10 aa, synthetic construct
  source 1..10: note = Synthetic Construct
GGGESGGGES

SEQ ID NO: 117 - protein, 10 aa, synthetic construct
  source 1..10: note = Synthetic Construct
GGGDSGGGGS

SEQ ID NO: 118 - protein, 10 aa, synthetic construct
  source 1..10: note = Synthetic Construct
GGGASGGGGS

SEQ ID NO: 119 - protein, 10 aa, synthetic construct
  source 1..10: note = Synthetic Construct
GGGESGGGGS

SEQ ID NO: 120 - protein, 6 aa, synthetic construct
  source 1..6: note = Synthetic Construct
ASTKGP

SEQ ID NO: 121 - protein, 13 aa, synthetic construct
  source 1..13: note = Synthetic Construct
ASTKGPSVFPLAP

SEQ ID NO: 122 - protein, 4 aa, synthetic construct
  source 1..4: note = Synthetic Construct
GGGP

SEQ ID NO: 123 - protein, 8 aa, synthetic construct
  source 1..8: note = Synthetic Construct
GGGGGGGP

SEQ ID NO: 124 - protein, 9 aa, synthetic construct
  source 1..9: note = Synthetic Construct
PAPNLLGGP

SEQ ID NO: 125 - protein, 6 aa, synthetic construct
  source 1..6: note = Synthetic Construct
GGGGGG

SEQ ID NO: 126 - protein, 12 aa, synthetic construct
  source 1..12: note = Synthetic Construct
GGGGGGGGGGGG

SEQ ID NO: 127 - protein, 8 aa, synthetic construct
  source 1..8: note = Synthetic Construct
APELPGGP

SEQ ID NO: 128 - protein, 8 aa, synthetic construct
  source 1..8: note = Synthetic Construct
SEPQPQPG

SEQ ID NO: 129 - protein, 15 aa, synthetic construct
  source 1..15: note = Synthetic Construct
GGGSSGGGSSGGGSS

SEQ ID NO: 130 - protein, 14 aa, synthetic construct
  source 1..14: note = Synthetic Construct
GGGGGGGGGSGGGS

SEQ ID NO: 131 - protein, 15 aa, synthetic construct
  source 1..15: note = Synthetic Construct
GGGGSGGGGGGGGGS

SEQ ID NO: 132 - protein, 15 aa, synthetic construct
  source 1..15: note = Synthetic Construct
GGSSSGGSSSGGSSS

SEQ ID NO: 133 - protein, 15 aa, synthetic construct
  source 1..15: note = Synthetic Construct
GSSSSGSSSSGSSSS

SEQ ID NO: 134 - protein, 15 aa, synthetic construct
  source 1..15: note = Synthetic Construct
GGGGAGGGGSGGGGS

SEQ ID NO: 135 - protein, 15 aa, synthetic construct
  source 1..15: note = Synthetic Construct
GGGGSGGGGAGGGGS

SEQ ID NO: 136 - protein, 15 aa, synthetic construct
  source 1..15: note = Synthetic Construct
GGGASGGGGSGGGGS

SEQ ID NO: 137 - protein, 15 aa, synthetic construct
  source 1..15: note = Synthetic Construct
GGGGSGGGASGGGGS

SEQ ID NO: 138 - protein, 15 aa, synthetic construct
  source 1..15: note = Synthetic Construct
GGGGSAGGGSGGGGS

SEQ ID NO: 139 - protein, 15 aa, synthetic construct
  source 1..15: note = Synthetic Construct
GGGGSGGGGSAGGGS

SEQ ID NO: 140 - protein, 15 aa, synthetic construct
  source 1..15: note = Synthetic Construct
GGGGSAGGGSAGGGS

SEQ ID NO: 141 - protein, 15 aa, synthetic construct
  source 1..15: note = Synthetic Construct
GGGGDGGGGSGGGGS

SEQ ID NO: 142 - protein, 15 aa, synthetic construct
  source 1..15: note = Synthetic Construct
GGGGSGGGGDGGGGS

SEQ ID NO: 143 - protein, 15 aa, synthetic construct
  source 1..15: note = Synthetic Construct
GGGGDGGGGDGGGGS

SEQ ID NO: 144 - protein, 15 aa, synthetic construct
  source 1..15: note = Synthetic Construct
GGGGEGGGGSGGGGS

SEQ ID NO: 145 - protein, 15 aa, synthetic construct
  source 1..15: note = Synthetic Construct
GGGGSGGGGEGGGGS

SEQ ID NO: 146 - protein, 15 aa, synthetic construct
  source 1..15: note = Synthetic Construct
GGGGEGGGGEGGGGS

SEQ ID NO: 147 - protein, 5 aa, synthetic construct
  source 1..5: note = Synthetic Construct
EVQLV

SEQ ID NO: 148 - protein, 11 aa, synthetic construct
  source 1..11: note = Synthetic Construct
WGQGTLVTVSS

SEQ ID NO: 149 - protein, 115 aa, synthetic construct [sequence omitted]

SEQ ID NO: 150 - protein, 114 aa, synthetic construct
  source 1..114: note = Synthetic Construct
EVQLVESGGGLVQTGGSLRLSCAASTSGSDFSGKKMAWYRQAPGNGREFVAIIFSNKVTDYADSVKGRFTISRDNAKKTVYLQMSSLTPTDTAVYYCHDQEISWGQGTQVTVSS

SEQ ID NO: 151 - protein, 122 aa, synthetic construct
  source 1..122: note = Synthetic Construct
EVQLVESGGGLVQAGGSLRLSCAASGTSVVINSMGWYRQAPGKQRELVATIDLSGTTNYADSAQGRFTISRDNAENLNLVYLQMNNLNPDDTAVYYCNALLSRAVSGSYVYWGQGTQVTVSS

SEQ ID NO: 152 - protein, 118 aa, synthetic construct
  source 1..118: note = Synthetic Construct
EVQLVESGGGLVQPGGSLRLSCTSRIGTISNIDLMNWYRQAPGKQREFVASLQSNGATNYADSVKGRFTISRDNAKNTLFLQMNSLNPEDTAVYFCHALLPRSPYNSWGQGTQVTVSS

SEQ ID NO: 153 - protein, 116 aa, synthetic construct
  source 1..116: note = Synthetic Construct
EVQLVESGGGLVQAGGSLRLSCAASSIIPNIYAMGWYRQAPGKQRELVASIENGLPANYADSVKGRFTISRDNAKNTVFLQMHSLKSEDTAVYYCYAFRPGVPTTWGQGTQVTVSS

SEQ ID NO: 154 - protein, 116 aa, synthetic construct
  source 1..116: note = Synthetic Construct
EVQLVESGGGLVQAGESLRLSCAASGSISAINAMGWYRQAPGKQREFVADITRAGVSDYADAVKGRFTISRDNAKNTFYLQMNDLKPEDTAVYYCDALLIAGGVYWGQGTQVTVSS

SEQ ID NO: 155 - protein, 126 aa, synthetic construct
  source 1..126: note = Synthetic Construct
EVQLVESGGGLVQAGGSLRLSCTASGRTISTTVMGWFRQAPGKEREFVAAVHWGDGNTVYADSVKGRFTISRDDAKNTVYLQLNYLKPEDTSVYYCAARPPTYVGTSRNSRSYDYWGQGTQVTVSS

SEQ ID NO: 156 - protein, 125 aa, synthetic construct
  source 1..125: note = Synthetic Construct
EVQLVESGGGLVQAGGSLRLSCVVSGRAIDRNAMGWFRQAPGKERESVAAISASSGNTYYSDSVTGRFTISRDNTKNTVYLQMNSLKPEDTAVYYCAAGSRGSWYLFDRREYDYWGQGTQVTVSS

SEQ ID NO: 157 - protein, 120 aa, synthetic construct
  source 1..120: note = Synthetic Construct
EVQLVESGGGLVQAGGSLRLTCTASETSFDINVMGWYRQAPGKQRELVAIITASGNTEYADSAKGRFTISRDNTKNTVAMQMNNLKPDDTAVYYCYVLLSGAVSGVYAHWGQGTQVTVSS

SEQ ID NO: 158 - protein, 130 aa, synthetic construct
  source 1..130: note = Synthetic Construct
EVQLVESGGGLVQAGGSLTLSCAASGRTDSRYAMGWFRQAPGKERELMAAISWSGRPTYYADSVKGRFTISRDNAKNTVSLQMNSLKPEDTAVYYCAYKRLPAWYTGSAYYSQESEYDYWGQGTQVTVSS

SEQ ID NO: 159 - protein, 118 aa, synthetic construct
  source 1..118: note = Synthetic Construct
EVQLVESGGGLVQPGGSLRLSCTSRIGTISNIDLMNWYRQAPGKQREFVASLQSTGTTDYADSVKGRFTISRDNAKNTLFLQMNSLNPEDTAVYYCHALIPRSPYNVWGQGTQVTVSS

SEQ ID NO: 160 - protein, 126 aa, synthetic construct
  source 1..126: note = Synthetic Construct
EVQLVESGGGLVQAGGSLRLSCTASGRTISTTVMAWFRQAPGKEREFVAADHWGDAGTVYADSVKGRFTISRDNAKNTVYLQMNYLKPEDTSVYYCAARPPTYVGTSRDSRAYDYWGQGTQVTVSS

SEQ ID NO: 161 - protein, 117 aa, synthetic construct
  source 1..117: note = Synthetic Construct
EVQLVESGGGLVQPGGSLRLSCAASESISSDSPMAWYRQAPGKQREMVARILPIGPPDYADAVKDRFSISRENAKNTVYLQMNSLKPEDTAVYYCNLLHLPSGLNYWGQGTQVTVSS

SEQ ID NO: 162 - protein, 109 aa, synthetic construct
  source 1..109: note = Synthetic Construct
EVQLVESGGDLVQAGGSLRLSCVASRSISSAMNWYRQPPGKQRELVALITRGFNTNYADSVKGRFTISRDNAKNTVYLQMNSLKPEDTGVYYCNSLNYWGQGTQVTVSS

SEQ ID NO: 163 - protein, 124 aa, synthetic construct
  source 1..124: note = Synthetic Construct
EVQLVESGGGLVQAGGSLRLSCAASGRTDSMWSMGWFRQAPGQEREFVAAISWSVGTYYEDSVKGRFTLSRDDDKDTAYLEMSDLKLEDTADYYCAASTRHGTNLVLPRDYDYWGQGTQVTVSS

SEQ ID NO: 164 - protein, 118 aa, synthetic construct
  source 1..118: note = Synthetic Construct
EVQLVESGGGLVQPGGSLRLSCTSRIGTISNIDLMNWYRQAPGKQREFVASLQSTGTTDYADSVKGRFTISRDNAKNTLFLQMNSLNPEDTAVYYCHALLPRSPYNAWGQGTQVTVSS

SEQ ID NO: 165 - protein, 116 aa, synthetic construct
  source 1..116: note = Synthetic Construct
EVQLVESGGGLVQAGGSLRLSCAASGIIPNIYAMGWYRQAPGKQRELVASIENGGSTNYADSVKGRFTISRDNARNTVFLQMHSLKSEDTAVYYCYAFRPGVPTDWGQGTQVTVSS

SEQ ID NO: 166 - protein, 118 aa, synthetic construct
  source 1..118: note = Synthetic Construct
EVQLVESGGGLVQAGGSLTLSCVASGRTFSNYRMGWFRQAPGAEREFVGTIYWSTGRSYYGDSVKGRFIISGDNAKNTIHLQMNSLKPEDTGVYYCASGPENSAFDSWGQGTQVTVSS

SEQ ID NO: 167 - protein, 124 aa, synthetic construct
  source 1..124: note = Synthetic Construct
EVQLVESGGGLVQAGDSLRLSCAASGRPFSSYTMGWFRQAPGKERDFVATISWSGGIKYYADSVEGRFSISRDNAKNMVYLQMNSLKPEDTAVYYCAATELRTWSRQTFEYDYWGQGTQVTVSS

SEQ ID NO: 168 - protein, 126 aa, synthetic construct
  source 1..126: note = Synthetic Construct
EVQLVESGGGLVQAGGSLRLSCTASGRTISTTVMAWFRQAPGKEREFVAAVHWGDESTVYADSVKGRFTISRDNAKNTVYLQMNYLKPEDTSVYYCAARPPTYVGSSRSSRAYDYWGQGTQVTVSS

SEQ ID NO: 169 - protein, 120 aa, synthetic construct
  source 1..120: note = Synthetic Construct
EVQLVESGGGLVQAGGSLRLSCVVSGSILDINVMAWYRQAPGKQREFVARITSGGDIDYADPVKGRFTISTNGAKNTVYLQMNSLKPEDTAAYYCNVLLSRSSAGRYTHWGQGTQVTVSS

SEQ ID NO: 170 - protein, 112 aa, synthetic construct
  source 1..112: note = Synthetic Construct
EVQLVESGGGLVQPGGSLRLSCAASGFPFSLYDMGWYRQAPEKQRESVAIITQSGSTDYADSVKGRFTISRDNAKNTLYLQMNSLKPEDTAVYYCRLVGVTWGQGTQVTVSS

SEQ ID NO: 171 - protein, 120 aa, synthetic construct
  source 1..120: note = Synthetic Construct
EVQLVESGGGLVQAGGSLTLSCAASGRTFSSYGIGWFRQAPGKEREFVAAISRTGQTTHYADSIRFTISRDNAKNTVYLQMNSLKPEDTAVYYCAARTGGPIYGSEYHYWGQGTQVTVSS

SEQ ID NO: 172 - protein, 124 aa, synthetic construct
  source 1..124: note = Synthetic Construct
EVQLVESGGGLVQAGDSLTLSCAASGRPFSSLTMGWFRQAPGKGREFVATTSWSGDIKYYADFVKGRFTISRDNAKNMVYLQMNSLKPEDTAVYYCAATLLRTWSRQTNEYEYWGQGTQVTVSS

SEQ ID NO: 173 - protein, 118 aa, synthetic construct
  source 1..118: note = Synthetic Construct
EVQLVESGGGLVQPGGSLRLSCTSRIGTISNIDLMNWYRQAPGKQREFVASLQSTGTTDYADSVRGRFTISRDNAKNTLFLQMNSLNPEDTAVYYCHALLPRSPYNVWGQGTQVTVSS

SEQ ID NO: 174 - protein, 132 aa, synthetic construct
  source 1..132: note = Synthetic Construct
EVQLVESGGGLVQAGGSLRLSCAASGRTFSGILSPYAVGWFRQAPGKGREFVSTITSGGSAIYTDSVKGRFTLSRDNAKDTVYLQMNSLKPEDTAVYYCAVRTRRYGSNLGEVPQENEYGYWGQGTQVTVSS

SEQ ID NO: 175 - protein, 120 aa, synthetic construct
  source 1..120: note = Synthetic Construct
EVQLVESGGGLVQAGGSLRLSCAAPETGATINVMAWYRQAPGKQRELVARVAIDNNTDYADHAKGRFTISRDNTKNTVYLQMNNLKPDDTAVYYCNVLLSRQISGSYGHWGQGTQVTVSS

SEQ ID NO: 176 - protein, 133 aa, synthetic construct
  source 1..133: note = Synthetic Construct
EVQLVESGGGLVQAGGSLTLSCAMSGGTRPFEDYVMAWFRQATGKEREFVATITWMGETTYYKDSVNGRFAISRDNAENTVALQMNSLEPEDTAVYFCAAHSRSSFSTSGGRYNPRPTEYDYWGQGTQVTVSS

SEQ ID NO: 177 - protein, 126 aa, synthetic construct
  source 1..126: note = Synthetic Construct
EVQLVESGGGLVQAGGSLRLSCTASGRTISTTVMGWFRQAPGKEREFVAAVHWGDEGTVYADSVKGRFTISRDNAKNTVYLQMNALKPEDTSVYYCAAKPPTYVGTSRSSRAYVYWGQGTQVTVSS

SEQ ID NO: 178 - protein, 120 aa, synthetic construct
  source 1..120: note = Synthetic Construct
EVQLVESGGGLVQAGDSLTLSCAASGSGFSINVMAWYRQAPGKQRDLVASMTIGGRTNYKDSLKGRFTISRDNTKNTAYLQMNSLKPEDTAVYYCYALLDRGIGGNYVYWGQGTQVTVSS

SEQ ID NO: 179 - protein, 125 aa, synthetic construct
  source 1..125: note = Synthetic Construct
EVQLVESGGGLVQAGGSLRLSCAASGLTFSDYYMGWFRQAPGKERDFLARIGKSGIGKSYADSVRGRFTISRDNAKNTVYLQMNNLKLEDTAVYYCAADRDIAYDARLTAEYDYWGQGTQVTVSS

SEQ ID NO: 180 - protein, 126 aa, synthetic construct
  source 1..126: note = Synthetic Construct
EVQLVESGGGLVQAGGSLRLSCTASGRTISTTVMGWFRQAPGKEREFVAAVHWGDESTVYADSVKGRFTISRDNAKNTVYLQMNYLKPEDTAVYYCAARPPTYVGTSRSSRAYDYWGQGTQVTVSS

SEQ ID NO: 181 - protein, 123 aa, synthetic construct
  source 1..123: note = Synthetic Construct
EVQLVESGGGLVQAGGSLRLSCAASVASETIVSINDMAWYRQAPGKQRELVASITIHNNRDYADSAKGRFTISRDDTKNTVYLQMTHLKPDDTAVYYCTVLLSRALSGSYRFWGQGTQVTVSS

SEQ ID NO: 182 - protein, 123 aa, synthetic construct
  source 1..123: note = Synthetic Construct
EVQLVESGGGLVQAGGSLRLSCTGSETSGTIFNINVMGWYRQAPGKQRELVAIMDIGGTTDYADSVKGRFTISRDNAKNTVYVQMNNLKSEDTAVYYCYCALDRAVAGRYTYWGQGTQVTVSS

SEQ ID NO: 183 - protein, 124 aa, synthetic construct
  source 1..124: note = Synthetic Construct
EVQLVESGGGLVQPGGSLRLSCEASGISLNDYNMGWFRQAPGKDREIVAALSRRSHGIYQSDSVKYRFSISRDNTKNMVSLQMDSLRPEDTAVYYCAADGDPYFTGRDMNPEYWGQGTQVTVSS

SEQ ID NO: 184 - protein, 124 aa, synthetic construct
  source 1..124: note = Synthetic Construct
EVQLVESGGGSVQAGGSLRLSCAFSGGRFSDYGMAWFRQGPGKEREFVSRISGNGRGTQYTDSVSGRFIISRDNDKNTVYLQMNDLKVEDTAIYYCARGSGPSSFNEGSVYDYWGQGTQVTVSS

SEQ ID NO: 185 - protein, 113 aa, synthetic construct
  source 1..113: note = Synthetic Construct
EVQLVESGGGLVQSGGSLTLSCVLSGSIFSSNTMGWHRQAPGKQREWVAITTSGGTTKYADSVKGRFTISRDNAKNTVYLRMNNLKPEDTGVYFCYASLAGIWGQGTQVTVSS

SEQ ID NO: 186 - protein, 120 aa, synthetic construct
  source 1..120: note = Synthetic Construct
EVQLVESGGGLVQAGGSLRLSCAAPETEATYNVMGWYRRAPGKQRELVATMTIDYNTNYADSAKGRFTISRDNTKNTVYLQMNNLRPDDTAVYYCRVDLSRQISGSYNYWGQGTQVTVSS

SEQ ID NO: 187 - protein, 125 aa, synthetic construct
  source 1..125: note = Synthetic Construct
EVQLVESGGGLVQPGESLRLSCAISGFAFTDVGMSWVRQAPGKGLEWVSSISSGSSITTYSDSVKGRFTISRDNARNTLFLQMNSLKPEDTAVYYCGRYYCTGLGCHPRRDSALWGQGTQVTVSS

SEQ ID NO: 188 - protein, 127 aa, synthetic construct
  source 1..127: note = Synthetic Construct
EVQLVESGGGLVQPGGSLRLSCRASGFTYSTAAMGWVRQAPGKGLEWVSSISSLGSDRKSADSVKGRFTISRDNAKNTLYLQMNSLKPEDTAVYYCARFISNRWSRDVHAPSDFGSRGQGTQVTVSS

SEQ ID NO: 189 - protein, 122 aa, synthetic construct
  source 1..122: note = Synthetic Construct
EVQLVESGGGSVPAGGSLRLSCAAFGFTFDNYAIAWFRQAPGKEREGVSCLSTNDGETYYADSVKGRFTISSDHAKNTVYLQMDSLRPEDTAVYYCAAAEGSWCHKYEYDYWGQGTQVTVSS

SEQ ID NO: 190 - protein, 125 aa, synthetic construct
  source 1..125: note = Synthetic Construct
EVQLVESGGGLVQAGESLRLSCAASGRTSDLYVVGWFRQTPGKEREFVAGIAWTGDASYYADSVEGRFTIARDNAENRIDLQMTSLKPEDTAVYYCAADSRARFERQRYNDMNYWGQGTQVTVSS

SEQ ID NO: 191 - protein, 120 aa, synthetic construct
  source 1..120: note = Synthetic Construct
EVQLVESGGGLVQAGGSLRLSCIASVTIADINVMGWYRQAPGKQREFVASIPTTGDKNYAESAKGRFTISRDNSQNTVAMQMNNLKPDDTAVYYCYVLLSRAVSGSYGHWGQGTQVTVSS

SEQ ID NO: 192 - protein, 123 aa, synthetic construct
  source 1..123: note = Synthetic Construct
EVQLVESGGGLVQVGGSLRLSCAASGSIVDIKVMGWYRQAPGNERELVALINDADDSEYSPSMRGRFTISRDNSKNTVYLQMNSLKPEDTAAYYCAADRDSSWFKSPYIPGSWGQGTQVTVSS

SEQ ID NO: 193 - protein, 120 aa, synthetic construct
  source 1..120: note = Synthetic Construct
EVQLVESGGGLVQAGGSLRLSCAAPEMGATINVMAWYRQAPGKQRELVARLPLDNNIDYGDFAKGRFTISRDITRNTVYLQMNNLKPDDTAVYYCNVLLSRQINGAYVHWGQGTQVTVSS

SEQ ID NO: 194 - protein, 120 aa, synthetic construct
  source 1..120: note = Synthetic Construct
EVQLVESGGGLVQAGGSLRLSCAASGIDGDINVMAWYRQAPGKQRELVASITIGGNTNYADSVKGRFTIARDNAKNRMSLEMNSLKSEDTAVYYCNTLLSRVHDGQYVFWGQGTQVTVSS

SEQ ID NO: 195 - protein, 125 aa, synthetic construct
  source 1..125: note = Synthetic Construct
EVQLVESGGGLVQAGGSLRLSCVASEDAFKTDTLGWFRQAPGEEREFVAAFVWAGGPFYADSVKGRFTISMDEDRNTVYLQMNSLKPEDTGVYYCAASLSRLRVGEITPRHMNYWGQGTQVTVSS

SEQ ID NO: 196 - protein, 126 aa, synthetic construct
  source 1..126: note = Synthetic Construct
EVQLVESGGGLVQAGGSLRLSCAASGRAFSDYAMAWFRQAPGKEREFVAGIGWSGGDTLYADSVRGRFTNSKDNAKNRMSLQMNSLKPEDTAVYYCAARQGQYIYSSMRSDSYDYWGQGTQVTVSS

SEQ ID NO: 197 - protein, 124 aa, synthetic construct
  source 1..124: note = Synthetic Construct
EVQLVESGGGLVQAGGSLRLSCAASGRTFSSSNMGWFRQAPGEEREFVTAIDWSGGRTYYADSVKGRFTISRDNAKNTVYLQMDSLKPEDTAVYYCAAQGSGLDWGYPWTYDYWGQGTQVTVSS

SEQ ID NO: 198 - protein, 115 aa, synthetic construct
  source 1..115: note = Synthetic Construct
EVQLVESGGGLVQPGGSLKLSCATSGSVLNIDSMAWYRQAPGKQRELVAEMLWGGTKNYGDSVKGRFTISGDADWGTELQMSSLKPEDTAVYYCNAVGRGFRDAWGQGTQVTVSS

SEQ ID NO: 199 - protein, 116 aa, synthetic construct
  source 1..116: note = Synthetic Construct
EVQLVESGGGLVQAGGSLRLSCVASGSGFGILDMGWYRQAPGSRRELVGYVTRDGTTNYGNSVKGRSIISEDITKNTVILQMNSLKPEDTAVYFCTAGLTNQPRAWGQGTQVTVSS

SEQ ID NO: 200 - protein, 119 aa, synthetic construct
  source 1..119: note = Synthetic Construct
EVQLVESGGGLVQPGGSLRLSCAASGSVSSINVMGWYRQTPGKQRELVAAINRGGSTNVADSVKGRFTISRDNAKNTVYLQMNSLKPEDTAVYYCNAEPYGLDWRYDYWGQGTQVTVSS

SEQ ID NO: 201 - protein, 129 aa, synthetic construct
  source 1..129: note = Synthetic Construct
EVQLVESGGGLEQAGGSLRLSCTASGGTDSIYQMGWFRQTPGKEREFVAAINWNYGGAYYPDSVKGRFTISRDKAKNIGFLQMNSLKPEDTAVYYCATSQTSVDAFSVPITTARRYQYWGQGTQVTVSS

SEQ ID NO: 202 - protein, 118 aa, synthetic construct
  source 1..118: note = Synthetic Construct
EVQLVESGGGLVQAGGSLTLSCVASGRTFSNYRMGWFRQAPGKEREFVGTIYWSTGRSYYGDSVKGRFIISGDNAKNTIHLQMNSLKPGDTGVYYCASGPEMSAFDSWGQGTQVTVSS

SEQ ID NO: 203 - protein, 127 aa, synthetic construct
  source 1..127: note = Synthetic Construct
EVQLVESGGGLVQPGGSLRLSCAASGFTLDDYAIGWFRQAPGKEREGVSCISSSDGSTYYGDSVKGRFTISRDNAKNTMYLQMNSLKPEDTAVYYCATGTPLSSYYGSCLDYDMAYWGQGTQVTVSS

SEQ ID NO: 204 - protein, 124 aa, synthetic construct
  source 1..124: note = Synthetic Construct
EVQLVESGGGLVQAGGSLRLSCAASGVTFSNYGMAWFRQAPEKEREFVARISSNGRRTEYADGVSGRFTISRDNAKNTVYLQMNGLKPEDTAVYYCARAAGPSGFHEQSIYDDWGQGTQVTVSS

SEQ ID NO: 205 - protein, 123 aa, synthetic construct
  source 1..123: note = Synthetic Construct
EVQLVESGGGLVQAGGSLRLSCAVSGRSISTYVAGWFRQGPGKEREFVALISRGGGDIQYSDSVKGRFTISRDNAKNAVYLQMNSLKPADTAVYYCSLDASFGSRLVSRWDYWGQGTQVTVSS

SEQ ID NO: 206 - protein, 128 aa, synthetic construct
  source 1..128: note = Synthetic Construct
EVQLVESGGGVVQAGDSLTLTCTAPVGTISDYGMGWFRQAPGKEREFVASISWGGMWTDYADSVKGRFTISRDNDKNAVYLRMNSLNAEDTAVYYCGRGRMYRGIGNSLAQPKSYGYWGQGTQVTVSS

SEQ ID NO: 207 - protein, 126 aa, synthetic construct
  source 1..126: note = Synthetic Construct
EVQLVESGGGLVQAGGSLRLSCAGSGFTSDDYAIAWFRQAPGKEREGVSCIGSGDGTTYYADSVKGRFIISSENAKKTVYLQMNSLKPEDTGIYYCAADLYPPADYALDHTWYDYWGQGTQVTVSS

SEQ ID NO: 208 - protein, 113 aa, synthetic construct
  source 1..113: note = Synthetic Construct
EVQLVESGGGVVQPGGSLRLSCVVSGSRFSLDTVGWHHQAPGKLRELVARIRDDGDTMYVASVKGRFIISRDDAKNTVYLQMNSLKPEDTGVYYCYFSRNGAWGQGTQVTVSS

SEQ ID NO: 209 - protein, 120 aa, synthetic construct
  source 1..120: note = Synthetic Construct
EVQLVESGGGLVQAGGSLRLSCGASGRISDINVMGWYRQAPGKQREMVADIDIRGYTNYADSVKGRFTVSRDNAETMYLEMNSLKPEDTAVYRCNALTSRDWGTGKYVYWGQGTQVTVSS

SEQ ID NO: 210 - protein, 111 aa, synthetic construct
  source 1..111: note = Synthetic Construct
EVQLVESGGDLVQVGGSLRLSCAFPGSMSSRNSVNWYRQPPGKQREWVATISVSGFTQYADSAKGRFTISRDSAKNTVHLQMNSLKPEDTGVYYCNYMDYWGQGTQVTVSS

SEQ ID NO: 211 - protein, 113 aa, synthetic construct
  source 1..113: note = Synthetic Construct
EVQLVESGGGVVRAGGSLKLSCTAAGTDINIVTVGWHRQAPGKHRELVATIVGSGSRTNYADSVKGRFTISRDNPKNTVYLQMNSLKPEDTAVYYCYATSIGWGQGTQVTVSS

SEQ ID NO: 212 - protein, 132 aa, synthetic construct
  source 1..132: note = Synthetic Construct
EVQLVESGGGLVQAGGSLRLSCAASGRTFSGILSAYAVGWFRQAPGKEREFVSTITSGGSTLSADSVKGRFTLSRDNAKDTVYLQMNSLKPEDTAVYYCAVRTWPYGSNRGEVPTENEYGHWGQGTQVTVSS

SEQ ID NO: 213 - protein, 118 aa, synthetic construct
  source 1..118: note = Synthetic Construct
EVQLVESGGGSVQAGGSLRLTCTASGNVRSIFTMAWYRQAPGKQRELVASAAKGGDTYYADSAKGRFTISRDDAKAIVSLQMNSLKPEDTAVYYCKTDGRPWFSEDYWGQGTQVTVSS

SEQ ID NO: 214 - protein, 111 aa, synthetic construct
  source 1..111: note = Synthetic Construct
EVQLVESGGGLVQVGDSMRLSCAVFGNIFTRDPVMWFRQPPGKQREWVATITPSGFANYADSVKGRFTISRYAANNTVHLQMNSLKPEDTGVYFCNFGTYWGQGTQVTVSS

SEQ ID NO: 215 - protein, 120 aa, synthetic construct
  source 1..120: note = Synthetic Construct
EVQLVESGGGLVQAGGSLRLSCAASKGAFNINVMAWYRQAPGKQRELVARVALGGTTDYADSVKGRFTISRNNAQDTVYLQMNSLKPEDTAVYYCNVLLDRGVRGSYAYWGQGTQVTVSS

SEQ ID NO: 216 - protein, 122 aa, synthetic construct
  source 1..122: note = Synthetic Construct
EVQLVESGGGLVQAGGSLRLSCAASGRTYSSYVIGWFRQAPGKEREFVASIRWAGGDSHYQESVKGRSTISKDNARNTVYLQMNSLKPEDTAVYYCAGAAPVPGQSYEWSSWGQGTQVTVSS

SEQ ID NO: 217 - protein, 121 aa, synthetic construct
  source 1..121: note = Synthetic Construct
EVQLVESGGGLVQAGGSLRLSCVASGSAFYVGPMAWYRQAPGKERESVASITKGGITNYADSVKGRFTISRDNAKNTVYLQMNSLKPEDTDVYVCNARVKLQEDRLFRDYWGQGTQVTVSS

SEQ ID NO: 218 - protein, 116 aa, synthetic construct
  source 1..116: note = Synthetic Construct
EVQLVESGGGMVQPGGSLRLSCVVSGASGNIDFVTVGWHRQAPGKHREMVAVITGDGTRNYRDSVKGRFSISRDNAKNTIYLQMNSLKPEDTAVYYCYMSNPISSWGQGTQVTVSS

SEQ ID NO: 219 - protein, 126 aa, synthetic construct
  source 1..126: note = Synthetic Construct
EVQLVESGGGLVQAGGSRRLSCAVSGRTLSSFGMGWFRQAPEKPREFVAAITWGQGGTFYADSVKGRFTISRDIVKNTVYLQMNDLKPDDTGLYFCVSAPHFHEAFPSRPPAYAYWGQGTQVTVSS

SEQ ID NO: 220 - protein, 122 aa, synthetic construct
  source 1..122: note = Synthetic Construct
EVQLVESGGGLVQAGGSLRLSCAASGRTYGSYVIGWFRQAPGKEREFVASIRWAGGDSHYGDPLKGRSTISKDNAKNTVYLQMNSLKPEDAAVYYCAGAAPVPGSSYEWTNWGQGTQVTVSS

SEQ ID NO: 221 - protein, 120 aa, synthetic construct
  source 1..120: note = Synthetic Construct
EVQLVESGGGLVQAGGSLRLSCAASGSISSVNTMGWYRQAPGKQRELVAFITSGDDTNYADSMKGRFTISRDNAKNTLYLQMNSLKPEDTAVYYCVATLGRSSSGTYTYWGQGTQVTVSS

SEQ ID NO: 222 - protein, 127 aa, synthetic construct
  source 1..127: note = Synthetic Construct
EVQLVESGGGLVQAGGSLRLSCAASLRTLDNYGVGWFRQTPGREREFVSAVSWNGDRTYYQDSVKGRFTISREYAKNTVYLQMDSLKPEDTAVYYCAVNMYGSTFPGLSVESHYDYWGQGTQVTVSS

SEQ ID NO: 223 - protein, 117 aa, synthetic construct
  source 1..117: note = Synthetic Construct
EVQLVESGGGLVQAGGSLRLSCAASGSIFSINAMAWYRQAQGKQRELVADITKNDITDYADSVKGRFTIARDNAKNTVDLQMNSLKPEDTAVYYCTAALSRHPYRSWGQGTQVTVSS

SEQ ID NO: 224 - protein, 122 aa, synthetic construct
  source 1..122: note = Synthetic Construct
EVQLVESGGGLVQAGGSLRLSCAAAGRSLSDYYIIWFRQPPGKEYEFVSSIRWNTGSTTYGDSVKGRFTISRDNAKSTVYLQMNSLKPEDTALYWCAAGLHLTPTSRTYNYRGQGTQVTVSS

SEQ ID NO: 225 - protein, 120 aa, synthetic construct
  source 1..120: note = Synthetic Construct
EVQLVESGGGLVQAGGSLRLSCAAPETIFTINSMGWYRQAPGKQRELVAFINLDGNTNYADSAKGRFTISRDNAENTVYLQMDNLKPDDTAVYYCNVLLSRAISGSYVHWGQGTQVTVSS

SEQ ID NO: 226 - protein, 132 aa, synthetic construct
  source 1..132: note = Synthetic Construct
EVQLVESGGGLVQPGGSLRLSCAASGRTFSGILSPYAVGWFRQAPGQGLEAVATITSGGSAIYTDSVKGRFTISRDNSKNTLYLQMNSLRAEDTAVYYCAVRTRRYGSNLGEVPQENEYGYWGQGTLVTVSS

SEQ ID NO: 227 - protein, 120 aa, synthetic construct
  source 1..120: note = Synthetic Construct
EVQLVESGGGLVQPGGSLRLSCAASEMGATINVMAWFRQAPGQGLEAVARLPLDNNIDYGDFAKGRFTISRDNSKNTLYLQMNSLRAEDTAVYYCNVLLSRQINGAYVHWGQGTLVTVSS

SEQ ID NO: 228 - protein, 126 aa, synthetic construct
  source 1..126: note = Synthetic Construct
EVQLVESGGGLVQPGGSLRLSCAASGRAFSDYAMAWFRQAPGQGLEAVAGIGWSGGDTLYADSVRGRFTISRDNSKNTLYLQMNSLRAEDTAVYYCAARQGQYIYSSMRSDSYDYWGQGTLVTVSS

SEQ ID NO: 229 - protein, 132 aa, synthetic construct
  source 1..132: note = Synthetic Construct
EVQLVESGGGLVQPGGSLRLSCAASGRTFSGILSPYAVGWFRQAPGQGREFVATITSGGSAIYTDSVKGRFTISRDNSKNTLYLQMNSLRAEDTAVYYCAVRTRRYGSNLGEVPQENEYGYWGQGTLVTVSS

SEQ ID NO: 230 - protein, 120 aa, synthetic construct
  source 1..120: note = Synthetic Construct
EVQLVESGGGLVQPGGSLRLSCAAPEMGATINVMAWYRQAPGQQRELVARLPLDNNIDYGDFAKGRFTISRDNSKNTLYLQMNSLRAEDTAVYYCNVLLSRQINGAYVHWGQGTLVTVSS

SEQ ID NO: 231 - protein, 126 aa, synthetic construct
  source 1..126: note = Synthetic Construct
EVQLVESGGGLVQPGGSLRLSCAASGRAFSDYAMAWFRQAPGQEREFVAGIGWSGGDTLYADSVRGRFTISRDNSKNTLYLQMNSLRAEDTAVYYCAARQGQYIYSSMRSDSYDYWGQGTLVTVSS

SEQ ID NO: 232 - protein, 132 aa, synthetic construct
  source 1..132: note = Synthetic Construct
EVQLVESGGGLVQPGGSLRLSCAASGRTFSGILSPYAVGWFRQAPGKGREFVSTITSGGSAIYTDSVKGRFTISRDNAKNSLYLQMNSLRAEDTAVYYCAVRTRRYGSNLGEVPQENEYGYWGQGTLVTVSS

SEQ ID NO: 233 - protein, 132 aa, synthetic construct
  source 1..132: note = Synthetic Construct
EVQLVESGGGLVQPGGSLRLSCAASGRTFSGILSPYAVGWFRQAPGKGLEFVSTITSGGSAIYTDSVKGRFTISRDNAKNSLYLQMNSLRAEDTAVYYCAVRTRRYGSNLGEVPQENEYGYWGQGTLVTVSS

SEQ ID NO: 234 - protein, 120 aa, synthetic construct
  source 1..120: note = Synthetic Construct
EVQLVESGGGLVKPGGSLRLSCAASEMGATINVMAWYRQAPGKQRELVSRLPLDNNIDYGDFAKGRFTISRDNAKNSLYLQMNSLRAEDTAVYYCNVLLSRQINGAYVHWGQGTLVTVSS

SEQ ID NO: 235 - protein, 120 aa, synthetic construct
  source 1..120: note = Synthetic Construct
EVQLVESGGGLVKPGGSLRLSCAASEMGATINVMAWYRQAPGKGLELVSRLPLDNNIDYGDFAKGRFTISRDNAKNSLYLQMNSLRAEDTAVYYCNVLLSRQINGAYVHWGQGTLVTVSS

SEQ ID NO: 236 - protein, 126 aa, synthetic construct
  source 1..126: note = Synthetic Construct
EVQLVESGGGLVQPGRSLRLSCAASGRAFSDYAMAWFRQAPGKEREFVSGIGWSGGDTLYADSVRGRFTISRDNAKNSLYLQMNSLRAEDTALYYCAARQGQYIYSSMRSDSYDYWGQGTLVTVSS

SEQ ID NO: 237 - protein, 126 aa, synthetic construct
  source 1..126: note = Synthetic Construct
EVQLVESGGGLVQPGRSLRLSCAASGRAFSDYAMAWFRQAPGKGLEFVSGIGWSGGDTLYADSVRGRFTISRDNAKNSLYLQMNSLRAEDTALYYCAARQGQYIYSSMRSDSYDYWGQGTLVTVSS

SEQ ID NO: 238 - protein, 128 aa, synthetic construct
  source 1..128: note = Synthetic Construct
EVQLVESGGGLVQPGGSLRLSCAASVGTISDYGMGWFRQAPGQGLEAVASISWGGMWTDYADSVKGRFTISRDNSKNTLYLQMNSLRAEDTAVYYCGRGRMYRGIGNSLAQPKSYGYWGQGTLVTVSS

SEQ ID NO: 239 - protein, 132 aa, synthetic construct
  source 1..132: note = Synthetic Construct
EVQLVESGGGLVQPGGSLRLSCAASGRTFSGILSAYAVGWFRQAPGQGLEAVATITSGGSTLSADSVKGRFTISRDNSKNTLYLQMNSLRAEDTAVYYCAVRTWPYGSNRGEVPTENEYGHWGQGTLVTVSS

SEQ ID NO: 240 - protein, 128 aa, synthetic construct
  source 1..128: note = Synthetic Construct
EVQLVESGGGLVQPGGSLRLSCAASVGTISDYGMGWFRQAPGQEREFVASISWGGMWTDYADSVKGRFTISRDNSKNTLYLQMNSLRAEDTAVYYCGRGRMYRGIGNSLAQPKSYGYWGQGTLVTVSS

SEQ ID NO: 241 - protein, 132 aa, synthetic construct
  source 1..132: note = Synthetic Construct
EVQLVESGGGLVQPGGSLRLSCAASGRTFSGILSAYAVGWFRQAPGQEREFVATITSGGSTLSADSVKGRFTISRDNSKNTLYLQMNSLRAEDTAVYYCAVRTWPYGSNRGEVPTENEYGHWGQGTLVTVSS

SEQ ID NO: 242 - protein, 128 aa, synthetic construct
  source 1..128: note = Synthetic Construct
EVQLVESGGGLVKPGGSLRLSCAASVGTISDYGMGWFRQAPGKEREFVSSISWGGMWTDYADSVKGRFTISRDNAKNSLYLQMNSLRAEDTAVYYCGRGRMYRGIGNSLAQPKSYGYWGQGTLVTVSS

SEQ ID NO: 243 - protein, 128 aa, synthetic construct
  source 1..128: note = Synthetic Construct
EVQLVESGGGLVKPGGSLRLSCAASVGTISDYGMGWFRQAPGKGLEFVSSISWGGMWTDYADSVKGRFTISRDNAKNSLYLQMNSLRAEDTAVYYCGRGRMYRGIGNSLAQPKSYGYWGQGTLVTVSS

SEQ ID NO: 244 - protein, 132 aa, synthetic construct
  source 1..132: note = Synthetic Construct
EVQLLESGGGLVQPGGSLRLSCAASGRTFSGILSAYAVGWFRQAPGKEREFVSTITSGGSTLSADSVKGRFTISRDNSKNTLYLQMNSLRAEDTAVYYCAVRTWPYGSNRGEVPTENEYGHWGQGTLVTVSS

SEQ ID NO: 245 - protein, 132 aa, synthetic construct
  source 1..132: note = Synthetic Construct
EVQLLESGGGLVQPGGSLRLSCAASGRTFSGILSAYAVGWFRQAPGKGLEFVSTITSGGSTLSADSVKGRFTISRDNSKNTLYLQMNSLRAEDTAVYYCAVRTWPYGSNRGEVPTENEYGHWGQGTLVTVSS

SEQ ID NO: 246 - protein, 132 aa, synthetic construct
  source 1..132: note = Synthetic Construct
QVQLVQSGAEVKKPGASVKVSCKASGRAFSDYAMAWVRQAPGQGLEWMGGIGWSGGDTLYADSVRGYTENFKDRVTMTRDTSTSTVYMELSSLRSEDTAVYYCARQGQYIYSSMRSDSYDYWGQGTLVTVSS

SEQ ID NO: 247 - protein, 132 aa, synthetic construct
  source 1..132: note = Synthetic Construct
QVQLVQSGAEVKKPGASVKVSCKASGRAFSDYAMAWFRQAPGQEREFMGGIGWSGGDTLYADSVRGYTENFKDRVTMTRDTSTSTVYMELSSLRSEDTAVYYCARQGQYIYSSMRSDSYDYWGQGTLVTVSS

SEQ ID NO: 248 - protein, 132 aa, synthetic construct
  source 1..132: note = Synthetic Construct
QVQLVQSGAEVKKPGASVKVSCKASGRAFSDYAMAWFRQAPGQGLEFMGGIGWSGGDTLYADSVRGYTENFKDRVTMTRDTSTSTVYMELSSLRSEDTAVYYCARQGQYIYSSMRSDSYDYWGQGTLVTVSS

SEQ ID NO: 249 - protein, 137 aa, synthetic construct
  source 1..137: note = Synthetic Construct
QVQLVQSGAEVKKPGASVKVSCKASVGTISDYGMGWVRQAPGQGLEWMGSISWGGMWTDYADSVKGYTENFKDRVTMTRDTSTSTVYMELSSLRSEDTAVYYCARGRGRMYRGIGNSLAQPKSYGYWGQGTLVTVSS

SEQ ID NO: 250 - protein, 137 aa, synthetic construct
  source 1..137: note = Synthetic Construct
QVQLVQSGAEVKKPGASVKVSCKASVGTISDYGMGWFRQAPGQEREFMGSISWGGMWTDYADSVKGYTENFKDRVTMTRDTSTSTVYMELSSLRSEDTAVYYCARGRGRMYRGIGNSLAQPKSYGYWGQGTLVTVSS

SEQ ID NO: 251 - protein, 137 aa, synthetic construct
  source 1..137: note = Synthetic Construct
QVQLVQSGAEVKKPGASVKVSCKASVGTISDYGMGWFRQAPGQGLEFMGSISWGGMWTDYADSVKGYTENFKDRVTMTRDTSTSTVYMELSSLRSEDTAVYYCARGRGRMYRGIGNSLAQPKSYGYWGQGTLVTVSS

SEQ ID NO: 252 - protein, 141 aa, synthetic construct
  source 1..141: note = Synthetic Construct
QVQLVQSGAEVKKPGASVKVSCKASGRTFSGILSAYAVGWVRQAPGQGLEWMGTITSGGSTLSADSVKGYTENFKDRVTMTRDTSTSTVYMELSSLRSEDTAVYYCARAVRTWPYGSNRGEVPTENEYGHWGQGTLVTVSS

SEQ ID NO: 253 - protein, 141 aa, synthetic construct
  source 1..141: note = Synthetic Construct
QVQLVQSGAEVKKPGASVKVSCKASGRTFSGILSAYAVGWFRQAPGQEREFMGTITSGGSTLSADSVKGYTENFKDRVTMTRDTSTSTVYMELSSLRSEDTAVYYCARAVRTWPYGSNRGEVPTENEYGHWGQGTLVTVSS

SEQ ID NO: 254 - protein, 141 aa, synthetic construct
  source 1..141: note = Synthetic Construct
QVQLVQSGAEVKKPGASVKVSCKASGRTFSGILSAYAVGWFRQAPGQGLEFMGTITSGGSTLSADSVKGYTENFKDRVTMTRDTSTSTVYMELSSLRSEDTAVYYCARAVRTWPYGSNRGEVPTENEYGHWGQGTLVTVSS

SEQ ID NO: 255 - protein, 126 aa, synthetic construct
  source 1..126: note = Synthetic Construct
EVQLVESGGGVVRPGGSLRLSFAASGRAFSDYAMAWFRQAPGKEREFVSGIGWSGGDTLYADSVRGRFTISRDNAKNSLYLQMNSLRAEDTALYHCAARQGQYIYSSMRSDSYDYWGQGTLVTVSS

SEQ ID NO: 256 - protein, 126 aa, synthetic construct
  source 1..126: note = Synthetic Construct
EVQLLESGGGLVQPGGSLRLSCAASGRAFSDYAMAWFRQAPGKEREFVSGIGWSGGDTLYADSVRGRFTISRDNSKNTLYLQMNSLRAEDTAVYYCAARQGQYIYSSMRSDSYDYWGQGTLVTVSS

SEQ ID NO: 257 - protein, 126 aa, synthetic construct
  source 1..126: note = Synthetic Construct
EVQLVESGGVVVQPGGSLRLSCAASGRAFSDYAMAWFRQAPGKEREFVSGIGWSGGDTLYADSVRGRFTISRDNSKNSLYLQMNSLRAEDTALYYCAARQGQYIYSSMRSDSYDYWGQGTLVTVSS

SEQ ID NO: 258 - protein, 128 aa, synthetic construct
  source 1..128: note = Synthetic Construct
EVQLVESGGGLVQPGGSLRLSCAASVGTISDYGMGWFRQAPGKEREFVSSISWGGMWTDYADSVKGRFTISRDNSKNTLYLQMNSLRAEDTAVYYCGRGRMYRGIGNSLAQPKSYGYWGQGTQVTVSS

SEQ ID NO: 259 - protein, 128 aa, synthetic construct
  source 1..128: note = Synthetic Construct
EVQLVESGGGLVQPGGSLRLSCAASVGTISDYGMGWFHQAPGKEREFVSSISWGGMWTDYADSVKGRFIISRDNSRNTLYLQTNSLRAEDTAVYYCGRGRMYRGIGNSLAQPKSYGYWGQGTLVTVSS

SEQ ID NO: 260 - protein, 128 aa, synthetic construct
  source 1..128: note = Synthetic Construct
EVQLVESGGGVVQPGRSLRLSCAASVGTISDYGMGWFRQAPGKEREFVASISWGGMWTDYADSVKGRFTISRDNSKNTLYLQMNSLRAEDTAVYYCGRGRMYRGIGNSLAQPKSYGYWGQGTQVTVSS

SEQ ID NO: 261 - protein, 132 aa, synthetic construct
  source 1..132: note = Synthetic Construct
EVQLVESGGGLVKPGGSLRLSCAASGRTFSGILSAYAVGWFRQAPGKEREFVSTITSGGSTLSADSVKGRFTISRDNAKNSLYLQMNSLRAEDTAVYYCAVRTWPYGSNRGEVPTENEYGHWGQGTQVTVSS

SEQ ID NO: 262 - protein, 132 aa, synthetic construct
  source 1..132: note = Synthetic Construct
EVQLVESGGGLVQPGGSLRLSCAASGRTFSGILSAYAVGWFRQAPGKEREFVSTITSGGSTLSADSVKGRFTISRDNSKNTLYVQMSSLRAEDTAVYYCAVRTWPYGSNRGEVPTENEYGHWGQGTQVTVSS

SEQ ID NO: 263 - protein, 132 aa, synthetic construct
  source 1..132: note = Synthetic Construct
EVQLVESGGGVVQPGGSLRLSCAASGRTFSGILSAYAVGWFRQAPGKEREFVSTITSGGSTLSADSVKGRFTISRDNSKNSLYLQMNSLRTEDTALYYCAVRTWPYGSNRGEVPTENEYGHWGQGTQVTVSS

SEQ ID NO: 264 - protein, 10 aa, synthetic construct
  source 1..10: note = Synthetic Construct
VGTISDYGMG

SEQ ID NO: 265 - protein, 14 aa, synthetic construct
  source 1..14: note = Synthetic Construct
GRTFSGILSAYAVG

SEQ ID NO: 266 - protein, 17 aa, synthetic construct
  source 1..17: note = Synthetic Construct
SISWGGMWTDYADSVKG

SEQ ID NO: 267 - protein, 16 aa, synthetic construct
  source 1..16: note = Synthetic Construct
TITSGGSTLSADSVKG

SEQ ID NO: 268 - protein, 21 aa, synthetic construct
  source 1..21: note = Synthetic Construct
GRGRMYRGIGNSLAQPKSYGY

SEQ ID NO: 269 - protein, 22 aa, synthetic construct
  source 1..22: note = Synthetic Construct
AVRTWPYGSNRGEVPTENEYGH

SEQ ID NO: 270 - protein, 132 aa, synthetic construct
  source 1..132: note = Synthetic Construct
EVQLVESGGGLVQAGGSLRLSCAASGRTFSGILSPYAVGWFRQAPGKGLEFVSTITSGGSAIYTDSVKGRFTISRDNAKNSLYLQMNSLRAEDTAVYYCAVRTRRYGSNLGEVPQENEYGYWGQGTLVTVSS

SEQ ID NO: 271 - protein, 132 aa, synthetic construct
  source 1..132: note = Synthetic Construct
EVQLVESGGGLVQPGGSLRLSCAASGRTFSGILSPYAVGWFRQAPGKGLEFVSTITSGGSAIYTDSVKGRFTLSRDNAKNSLYLQMNSLRAEDTAVYYCAVRTRRYGSNLGEVPQENEYGYWGQGTLVTVSS

SEQ ID NO: 272 - protein, 132 aa, synthetic construct
  source 1..132: note = Synthetic Construct
EVQLVESGGGLVQPGGSLRLSCAASGRTFSGILSPYAVGWFRQAPGKGLEFVSTITSGGSAIYTDSVKGRFTISRDNAKNSLYLQMNSLKAEDTAVYYCAVRTRRYGSNLGEVPQENEYGYWGQGTLVTVSS

SEQ ID NO: 273 - protein, 132 aa, synthetic construct
  source 1..132: note = Synthetic Construct
EVQLVESGGGLVQPGGSLRLSCAASGRTFSGILSPYAVGWFRQAPGKGLEFVSTITSGGSAIYTDSVKGRFTISRDNAKNSLYLQMNSLRPEDTAVYYCAVRTRRYGSNLGEVPQENEYGYWGQGTLVTVSS

SEQ ID NO: 274 - protein, 126 aa, synthetic construct
  source 1..126: note = Synthetic Construct
EVQLVESGGGLVQAGGSLRLSCAASGRAFSDYAMAWFRQAPGQEREFVAGIGWSGGDTLYADSVRGRFTISRDNSKNTLYLQMNSLRAEDTAVYYCAARQGQYIYSSMRSDSYDYWGQGTLVTVSS

SEQ ID NO: 275 - protein, 126 aa, synthetic construct
  source 1..126: note = Synthetic Construct
EVQLVESGGGLVQPGGSLRLSCAASGRAFSDYAMAWFRQAPGKEREFVAGIGWSGGDTLYADSVRGRFTISRDNSKNTLYLQMNSLRAEDTAVYYCAARQGQYIYSSMRSDSYDYWGQGTLVTVSS

SEQ ID NO: 276 - protein, 126 aa, synthetic construct
  source 1..126: note = Synthetic Construct
EVQLVESGGGLVQPGGSLRLSCAASGRAFSDYAMAWFRQAPGQEREFVAGIGWSGGDTLYADSVRGRFTISKDNSKNTLYLQMNSLRAEDTAVYYCAARQGQYIYSSMRSDSYDYWGQGTLVTVSS

SEQ ID NO: 277 - protein, 126 aa, synthetic construct
  source 1..126: note = Synthetic Construct
EVQLVESGGGLVQPGGSLRLSCAASGRAFSDYAMAWFRQAPGQEREFVAGIGWSGGDTLYADSVRGRFTISRDNSKNRLYLQMNSLRAEDTAVYYCAARQGQYIYSSMRSDSYDYWGQGTLVTVSS

SEQ ID NO: 278 - protein, 126 aa, synthetic construct
  source 1..126: note = Synthetic Construct
EVQLVESGGGLVQPGGSLRLSCAASGRAFSDYAMAWFRQAPGQEREFVAGIGWSGGDTLYADSVRGRFTISRDNSKNTLSLQMNSLRAEDTAVYYCAARQGQYIYSSMRSDSYDYWGQGTLVTVSS

SEQ ID NO: 279 - protein, 126 aa, synthetic construct
  source 1..126: note = Synthetic Construct
EVQLVESGGGLVQPGGSLRLSCAASGRAFSDYAMAWFRQAPGQEREFVAGIGWSGGDTLYADSVRGRFTISRDNSKNTLYLQMNSLKAEDTAVYYCAARQGQYIYSSMRSDSYDYWGQGTLVTVSS

SEQ ID NO: 280 - protein, 126 aa, synthetic construct
  source 1..126: note = Synthetic Construct
EVQLVESGGGLVQPGGSLRLSCAASGRAFSDYAMAWFRQAPGQEREFVAGIGWSGGDTLYADSVRGRFTISRDNSKNTLYLQMNSLRPEDTAVYYCAARQGQYIYSSMRSDSYDYWGQGTLVTVSS

SEQ ID NO: 281 - protein, 132 aa, synthetic construct
  source 1..132: note = Synthetic Construct
EVQLVESGGGLVQAGGSLRLSCAASGRTFSGILSPYAVGWFRQAPGKGREFVSTITSGGSAIYTDSVKGRFTLSRDNAKDTVYLQMNSLKPEDTAVYYCHVRTRRYGSNLGEVPQENEYGYWGQGTQVTVSS

SEQ ID NO: 282 - protein, 132 aa, synthetic construct
  source 1..132: note = Synthetic Construct
EVQLVESGGGLVQAGGSLRLSCAASGRTFSGILSPYAVGWFRQAPGKGREFVSTITSGGSAIYTDSVKGRFTLSRDNAKDTVYLQMNSLKPEDTAVYYCAVRTRRHGSNLGEVPQENEYGYWGQGTQVTVSS

SEQ ID NO: 283 - protein, 120 aa, synthetic construct
  source 1..120: note = Synthetic Construct
EVQLVESGGGLVQAGGSLRLSCAAPEMGATINVMAWYRQAPGKQRELVARLPHDNNIDYGDFAKGRFTISRDITRNTVYLQMNNLKPDDTAVYYCNVLLSRQINGAYVHWGQGTQVTVSS

SEQ ID NO: 284 - protein, 120 aa, synthetic construct
  source 1..120: note = Synthetic Construct
EVQLVESGGGLVQAGGSLRLSCAAPEMGATINVMAWYRQAPGKQRELVARLPLHNNIDYGDFAKGRFTISRDITRNTVYLQMNNLKPDDTAVYYCNVLLSRQINGAYVHWGQGTQVTVSS

SEQ ID NO: 285 - protein, 120 aa, synthetic construct
  source 1..120: note = Synthetic Construct
EVQLVESGGGLVQAGGSLRLSCAAPEMGATINVMAWYRQAPGKQRELVARLPLDNNIDYGDFAKGRFTISRDITRNTVYLQMNNLKPDDTAVYYCHVLLSRQINGAYVHWGQGTQVTVSS

SEQ ID NO: 286 - protein, 120 aa, synthetic construct
  source 1..120: note = Synthetic Construct
EVQLVESGGGLVQAGGSLRLSCAAPEMGATINVMAWYRQAPGKQRELVARLPLDNNIDYGDFAKGRFTISRDITRNTVYLQMNNLKPDDTAVYYCNVHLSRQINGAYVHWGQGTQVTVSS

SEQ ID NO: 287 - protein, 120 aa, synthetic construct
  source 1..120: note = Synthetic Construct
EVQLVESGGGLVQAGGSLRLSCAAPEMGATINVMAWYRQAPGKQRELVARLPLDNNIDYGDFAKGRFTISRDITRNTVYLQMNNLKPDDTAVYYCNVLLSRQINGAHVHWGQGTQVTVSS

SEQ ID NO: 288 - protein, 126 aa, synthetic construct
  source 1..126: note = Synthetic Construct
EVQLVESGGGLVQAGGSLRLSCAASGRHFSDYAMAWFRQAPGKEREFVAGIGWSGGDTLYADSVRGRFTNSKDNAKNRMSLQMNSLKPEDTAVYYCAARQGQYIYSSMRSDSYDYWGQGTQVTVSS

SEQ ID NO: 289 - protein, 126 aa, synthetic construct
  source 1..126: note = Synthetic Construct
EVQLVESGGGLVQAGGSLRLSCAASGRAHSDYAMAWFRQAPGKEREFVAGIGWSGGDTLYADSVRGRFTNSKDNAKNRMSLQMNSLKPEDTAVYYCAARQGQYIYSSMRSDSYDYWGQGTQVTVSS

SEQ ID NO: 290 - protein, 126 aa, synthetic construct
  source 1..126: note = Synthetic Construct
EVQLVESGGGLVQAGGSLRLSCAASGRAFSDYAMAWFRQAPGKEREFVAGIGWSGGDTHYADSVRGRFTNSKDNAKNRMSLQMNSLKPEDTAVYYCAARQGQYIYSSMRSDSYDYWGQGTQVTVSS

SEQ ID NO: 291 - protein, 126 aa, synthetic construct
  source 1..126: note = Synthetic Construct
EVQLVESGGGLVQAGGSLRLSCAASGRAFSDYAMAWFRQAPGKEREFVAGIGWSGGDTLYADSVRGRFTNSKDNAKNRMSLQMNSLKPEDTAVYYCAARQGQHIYSSMRSDSYDYWGQGTQVTVSS

SEQ ID NO: 292 - protein, 132 aa, synthetic construct
  source 1..132: note = Synthetic Construct
EVQLVESGGGLVQAGGSLRLSCAASGRTFSGILSHYAVGWFRQAPGKEREFVSTITSGGSTLSADSVKGRFTLSRDNAKDTVYLQMNSLKPEDTAVYYCAVRTWPYGSNRGEVPTENEYGHWGQGTQVTVSS

SEQ ID NO: 293 - protein, 132 aa, synthetic construct
  source 1..132: note = Synthetic Construct
EVQLVESGGGLVQPGGSLRLSCAASGRTFSGILSPYAVGWFRQAPGKGLEFVSTITSGGSAIYTDSVKGRFTISRDNAKNSLYLQMNSLRAEDTAVYYCAVRTRRHGSNLGEVPQENEYGYWGQGTLVTVSS

SEQ ID NO: 294 - protein, 126 aa, synthetic construct
  source 1..126: note = Synthetic Construct
EVQLVESGGGLVQPGGSLRLSCAASGRAFSDYAMAWFRQAPGQEREFVAGIGWSGGDTHYADSVRGRFTISRDNSKNTLYLQMNSLRAEDTAVYYCAARQGQYIYSSMRSDSYDYWGQGTLVTVSS

SEQ ID NO: 295 - protein, 126 aa, synthetic construct
  source 1..126: note = Synthetic Construct
EVQLVESGGGLVQPGGSLRLSCAASGRAFSDYAMAWFRQAPGQEREFVAGIGWSGGDTLYADSVRGRFTISRDNSKNTLYLQMNSLRAEDTAVYYCAARQGQHIYSSMRSDSYDYWGQGTLVTVSS

SEQ ID NO: 296 - protein, 126 aa, synthetic construct
  source 1..126: note = Synthetic Construct
EVQLVESGGGLVQPGGSLRLSCAASGRAHSDYAMAWFRQAPGQEREFVAGIGWSGGDTHYADSVRGRFTISRDNSKNTLYLQMNSLRAEDTAVYYCAARQGQYIYSSMRSDSYDYWGQGTLVTVSS

SEQ ID NO: 297 - protein, 126 aa, synthetic construct
  source 1..126: note = Synthetic Construct
EVQLVESGGGLVQPGGSLRLSCAASGRAFSDYAMAWFRQAPGQEREFVAGIGWSGGDTHYADSVRGRFTISRDNSKNTLYLQMNSLRAEDTAVYYCAARQGQHIYSSMRSDSYDYWGQGTLVTVSS

SEQ ID NO: 298 - protein, 126 aa, synthetic construct
  source 1..126: note = Synthetic Construct
EVQLVESGGGLVQPGGSLRLSCAASGRHFSDYAMAWFRQAPGQEREFVAGIGWSGGDTHYADSVRGRFTISRDNSKNTLYLQMNSLRAEDTAVYYCAARQGQYIYSSMRSDSYDYWGQGTLVTVSS

SEQ ID NO: 299 - protein, 126 aa, synthetic construct
  source 1..126: note = Synthetic Construct
EVQLVESGGGLVQPGGSLRLSCAASGRHFSDYAMAWFRQAPGQEREFVAGIGWSGGDTLYADSVRGRFTISRDNSKNTLYLQMNSLRAEDTAVYYCAARQGQHIYSSMRSDSYDYWGQGTLVTVSS

SEQ ID NO: 300 - protein, 126 aa, synthetic construct
  source 1..126: note = Synthetic Construct
EVQLVESGGGLVQPGGSLRLSCAASGRAHSDYAMAWFRQAPGQEREFVAGIGWSGGDTLYADSVRGRFTISRDNSKNTLYLQMNSLRAEDTAVYYCAARQGQHIYSSMRSDSYDYWGQGTLVTVSS

SEQ ID NO: 301 - protein, 126 aa, synthetic construct
  source 1..126: note = Synthetic Construct
EVQLVESGGGLVQPGGSLRLSCAASGRHHSDYAMAWFRQAPGQEREFVAGIGWSGGDTHYADSVRGRFTISRDNSKNTLYLQMNSLRAEDTAVYYCAARQGQYIYSSMRSDSYDYWGQGTLVTVSS

SEQ ID NO: 302 - protein, 126 aa, synthetic construct
  source 1..126: note = Synthetic Construct
EVQLVESGGGLVQPGGSLRLSCAASGRHHSDYAMAWFRQAPGQEREFVAGIGWSGGDTLYADSVRGRFTISRDNSKNTLYLQMNSLRAEDTAVYYCAARQGQHIYSSMRSDSYDYWGQGTLVTVSS

SEQ ID NO: 303 - protein, 126 aa, synthetic construct
  source 1..126: note = Synthetic Construct
EVQLVESGGGLVQPGGSLRLSCAASGRAHSDYAMAWFRQAPGQEREFVAGIGWSGGDTHYADSVRGRFTISRDNSKNTLYLQMNSLRAEDTAVYYCAARQGQHIYSSMRSDSYDYWGQGTLVTVSS

SEQ ID NO: 304 - protein, 126 aa, synthetic construct
  source 1..126: note = Synthetic Construct
EVQLVESGGGLVQPGGSLRLSCAASGRHFSDYAMAWFRQAPGQEREFVAGIGWSGGDTHYADSVRGRFTISRDNSKNTLYLQMNSLRAEDTAVYYCAARQGQHIYSSMRSDSYDYWGQGTLVTVSS

SEQ ID NO: 305 - protein, 262 aa, synthetic construct
  source 1..262: note = Synthetic Construct
EVQLVESGGGLVQPGGSLRLSCAASGRTFSGILSPYAVGWFRQAPGKGLEFVSTITSGGSAIYTDSVKGRFTISRDNAKNSLYLQMNSLRAEDTAVYYCAVRTRRYGSNLGEVPQENEYGYWGQGTLVTVSSGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 306 - protein, 267 aa, synthetic construct
  source 1..267: note = Synthetic Construct
EVQLVESGGGLVQPGGSLRLSCAASGRTFSGILSPYAVGWFRQAPGKGLEFVSTITSGGSAIYTDSVKGRFTISRDNAKNSLYLQMNSLRAEDTAVYYCAVRTRRYGSNLGEVPQENEYGYWGQGTLVTVSSGGGGSGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 307 - protein, 272 aa, synthetic construct
  source 1..272: note = Synthetic Construct
EVQLVESGGGLVQPGGSLRLSCAASGRTFSGILSPYAVGWFRQAPGKGLEFVSTITSGGSAIYTDSVKGRFTISRDNAKNSLYLQMNSLRAEDTAVYYCAVRTRRYGSNLGEVPQENEYGYWGQGTLVTVSSGGGGSGGGGSGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 308 - protein, 277 aa, synthetic construct
  source 1..277: note = Synthetic Construct
EVQLVESGGGLVQPGGSLRLSCAASGRTFSGILSPYAVGWFRQAPGKGLEFVSTITSGGSAIYTDSVKGRFTISRDNAKNSLYLQMNSLRAEDTAVYYCAVRTRRYGSNLGEVPQENEYGYWGQGTLVTVSSGGGGSGGGGSGGGGSGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 309 - protein, 262 aa, synthetic construct
  source 1..262: note = Synthetic Construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSGGGGSGGGGSEVQLVESGGGLVQPGGSLRLSCAASGRTFSGILSPYAVGWFRQAPGKGLEFVSTITSGGSAIYTDSVKGRFTISRDNAKNSLYLQMNSLRAEDTAVYYCAVRTRRYGSNLGEVPQENEYGYWGQGTLVTVSS

SEQ ID NO: 310 - protein, 267 aa, synthetic construct
  source 1..267: note = Synthetic Construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSGGGGSGGGGSGGGGSEVQLVESGGGLVQPGGSLRLSCAASGRTFSGILSPYAVGWFRQAPGKGLEFVSTITSGGSAIYTDSVKGRFTISRDNAKNSLYLQMNSLRAEDTAVYYCAVRTRRYGSNLGEVPQENEYGYWGQGTLVTVSS

SEQ ID NO: 311 - protein, 272 aa, synthetic construct
  source 1..272: note = Synthetic Construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSGGGGSGGGGSGGGGSGGGGSEVQLVESGGGLVQPGGSLRLSCAASGRTFSGILSPYAVGWFRQAPGKGLEFVSTITSGGSAIYTDSVKGRFTISRDNAKNSLYLQMNSLRAEDTAVYYCAVRTRRYGSNLGEVPQENEYGYWGQGTLVTVSS

SEQ ID NO: 312 - protein, 277 aa, synthetic construct
  source 1..277: note = Synthetic Construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSGGGGSGGGGSGGGGSGGGGSGGGGSEVQLVESGGGLVQPGGSLRLSCAASGRTFSGILSPYAVGWFRQAPGKGLEFVSTITSGGSAIYTDSVKGRFTISRDNAKNSLYLQMNSLRAEDTAVYYCAVRTRRYGSNLGEVPQENEYGYWGQGTLVTVSS

SEQ ID NO: 313 - protein, 256 aa, synthetic construct
  source 1..256: note = Synthetic Construct
EVQLVESGGGLVQPGGSLRLSCAASGRAFSDYAMAWFRQAPGQEREFVAGIGWSGGDTLYADSVRGRFTISRDNSKNTLYLQMNSLRAEDTAVYYCAARQGQYIYSSMRSDSYDYWGQGTLVTVSSGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 314 - protein, 261 aa, synthetic construct
  source 1..261: note = Synthetic Construct
EVQLVESGGGLVQPGGSLRLSCAASGRAFSDYAMAWFRQAPGQEREFVAGIGWSGGDTLYADSVRGRFTISRDNSKNTLYLQMNSLRAEDTAVYYCAARQGQYIYSSMRSDSYDYWGQGTLVTVSSGGGGSGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 315 - protein, 266 aa, synthetic construct
  source 1..266: note = Synthetic Construct
EVQLVESGGGLVQPGGSLRLSCAASGRAFSDYAMAWFRQAPGQEREFVAGIGWSGGDTLYADSVRGRFTISRDNSKNTLYLQMNSLRAEDTAVYYCAARQGQYIYSSMRSDSYDYWGQGTLVTVSSGGGGSGGGGSGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 316 - protein, 270 aa, synthetic construct
  source 1..270: note = Synthetic Construct
EVQLVESGGGLVQPGGSLRLSCAASGRAFSDYAMAWFRQAPGQEREFVAGIGWSGGDTLYADSVRGRFTISRDNSKNTLYLQMNSLRAEDTAVYYCAARQGQYIYSSMRSDSYDYWGQGTLVTVSSGGGGSGGGGSGGGGSGGGGSGGGGSGGGGSEVQLLESGGGLVQPGGSLRLSCAASGFTFRSFGMSWVRQAPGKGPEWVSSISGSGSDTLYADSVKGRFTISRDNSNTLYLQMNSLRPEDTAVYYCTIGGSLSRSSQGTLVTVSS

SEQ ID NO: 317 - protein, 256 aa, synthetic construct
  source 1..256: note = Synthetic Construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSGGGGSGGGGSEVQLVESGGGLVQPGGSLRLSCAASGRAFSDYAMAWFRQAPGQEREFVAGIGWSGGDTLYADSVRGRFTISRDNSKNTLYLQMNSLRAEDTAVYYCAARQGQYIYSSMRSDSYDYWGQGTLVTVSS

SEQ ID NO: 318 - protein, 261 aa, synthetic construct
  source 1..261: note = Synthetic Construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSGGGGSGGGGSGGGGSEVQLVESGGGLVQPGGSLRLSCAASGRAFSDYAMAWFRQAPGQEREFVAGIGWSGGDTLYADSVRGRFTISRDNSKNTLYLQMNSLRAEDTAVYYCAARQGQYIYSSMRSDSYDYWGQGTLVTVSS

SEQ ID NO: 319 - protein, 266 aa, synthetic construct
  source 1..266: note = Synthetic Construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSGGGGSGGGGSGGGGSGGGGSEVQLVESGGGLVQPGGSLRLSCAASGRAFSDYAMAWFRQAPGQEREFVAGIGWSGGDTLYADSVRGRFTISRDNSKNTLYLQMNSLRAEDTAVYYCAARQGQYIYSSMRSDSYDYWGQGTLVTVSS

SEQ ID NO: 320 - protein, 271 aa, synthetic construct
  source 1..271: note = Synthetic Construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSGGGGSGGGGSGGGGSGGGGSGGGGSEVQLVESGGGLVQPGGSLRLSCAASGRAFSDYAMAWFRQAPGQEREFVAGIGWSGGDTLYADSVRGRFTISRDNSKNTLYLQMNSLRAEDTAVYYCAARQGQYIYSSMRSDSYDYWGQGTLVTVSS

SEQ ID NO: 321 - protein, 9 aa, synthetic construct
  source 1..9: note = Synthetic Construct
GGGGGGGGP

SEQ ID NO: 322 - protein, 117 aa, synthetic construct
  source 1..117: note = Synthetic Construct
LEQVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 323 - protein, 7 aa, synthetic construct
  source 1..7: note = Synthetic Construct
PNLLGGP

SEQ ID NO: 324 - protein, 6 aa, synthetic construct
  source 1..6: note = Synthetic Construct
HHHHHH

SEQ ID NO: 325 - protein, 16 aa, synthetic construct
  source 1..16: note = Synthetic Construct
RLPLDNNIDYGDFAKG

SEQ ID NO: 326 - protein, 14 aa, synthetic construct
  source 1..14: note = Synthetic Construct
NVLLSRQINGAYVH

SEQ ID NO: 327 - protein, 10 aa, synthetic construct
  source 1..10: note = Synthetic Construct
EMGATINVMA

(19) United States
(12) Patent Application Publication
(10) Pub. No.: US 2025/0163138 A1
(43) Pub. Date: May 22, 2025

(54) POLYPEPTIDES THAT BIND COMPLEMENT COMPONENT C5 OR SERUM ALBUMIN AND FUSION PROTEINS THEREOF

(71) Applicant: Alexion Pharmaceuticals, Inc., Boston, MA (US)

(72) Inventors: Bridget PUFFER, South Weymouth, MA (US); Julian CHANDLER, Guilford, CT (US); Nimish GERA, Waltham, MA (US); Douglas L. SHERIDAN, Branford, CT (US); Siddharth JINDAL, Hamden, CT (US); Paul P. TAMBURINI, Kensington, CT (US)

(21) Appl. No.: 19/007,782

(22) Filed: Jan. 2, 2025

Related U.S. Application Data
(62) Division of application No. 17/938,756, filed on Oct. 7, 2022, now Pat. No. 12,221,473.
Application No. 17/938,756 is a continuation of application No. 16/629,687, filed on Jan. 9, 2020, now Pat. No. 11,498,960.
(60) Provisional application No. 62/531,215, filed on Jul. 11, 2017.

(51) Int. Cl. C07K 16/18; A61K 38/47; A61K 39/00; A61K 39/395

(52) U.S. Cl. CPC C07K 16/18; A61K 38/47; A61K 39/3955; C12Y 302/01035; A61K 2039/505; C07K 2317/24; C07K 2317/31; C07K 2317/565; C07K 2317/569; C07K 2317/92; C07K 2317/94

(57) ABSTRACT

The disclosure provides engineered polypeptides that specifically bind to human complement component C5 and/or serum albumin. The disclosure also provides fusion proteins comprising such engineered polypeptides, wherein such fusion proteins may be multivalent and multi-specific fusion proteins. The disclosure further provides nucleic acid molecules that encode such engineered polypeptides or fusion proteins, and methods of making such engineered polypeptides or fusion proteins. The disclosure further provides pharmaceutical compositions that comprise such engineered polypeptides or fusion proteins, and methods of treatment using such engineered polypeptides or fusion proteins.

### RELATED INFORMATION PARAGRAPH

This application claims the benefit of the priority date of U.S. Provisional Application No. 62/531,215, filed on Jul. 11, 2017, the content of which is hereby incorporated by reference in its entirety.

### SEQUENCE LISTING

The instant application contains a Sequence Listing which has been submitted electronically in XML format and is hereby incorporated by reference in its entirety. Said XML copy, created on Sep. 30, 2022, is named 51196-005003_Sequence_Listing_9_30_22 and is 378,627 bytes in size.

### BACKGROUND

Complement component 5 (C5) is the fifth component of complement, which plays an important role in inflammatory and cell killing processes. An activation peptide, C5a, which is an anaphylatoxin that possesses potent spasmogenic and chemotactic activity, is derived from the alpha polypeptide via cleavage with a C5-convertase. The C5b macromolecular cleavage product can form a complex with the C6 complement component, and this complex is the basis for formation of the membrane attack complex (MAC), which includes additional complement components.

Improperly regulated C5 can lead to immuno-compromised patients or disorders characterized by excessive cellular degradation (e.g., hemolytic disorders cause by C5-mediated hemolysis).

As misregulated C5 can lead to severe and devastating phenotypes, modulators of C5 activity with favorable pharmaceutical properties (e.g., half-life) are needed.

### SUMMARY

The disclosure provides engineered polypeptides that specifically bind to complement component C5 or serum albumin, wherein such engineered polypeptides may be sdAbs or Ig variable domains. In some embodiments, the engineered polypeptides do not significantly reduce or inhibit the binding of serum albumin to FcRn or do not significantly reduce the half-life of serum albumin. The disclosure also provides fusion proteins comprising such engineered polypeptides, wherein such fusion proteins may be multivalent and multi-specific fusion proteins. The disclosure further provides nucleic acid molecules that encode such engineered polypeptides or fusion proteins, and methods of making such engineered polypeptides or fusion proteins. The disclosure further provides pharmaceutical compositions that comprise such engineered polypeptides or fusion proteins, and methods of treatment using such engineered polypeptides or fusion proteins.

In one embodiment, the disclosure is directed to a fusion protein comprising an engineered polypeptide that specifically binds to human complement component C5 and an engineered polypeptide that specifically binds to human serum albumin, wherein the engineered polypeptide that specifically binds to human complement component C5 is fused to the polypeptide that specifically binds to human serum albumin either directly or via a peptide linker. In a particular embodiment, the C-terminal residue of the polypeptide that specifically binds to human serum albumin is fused either directly or via a linker to the N-terminal residue of the polypeptide that specifically binds to human complement component C5. In a particular embodiment, the C-terminal residue of the polypeptide that specifically binds to human complement component C5 is fused either directly or via a linker to the N-terminal residue of the polypeptide that specifically binds to human serum albumin. In a particular embodiment, the polypeptide that specifically binds to human complement component C5 comprises an amino acid sequence selected from the group consisting of SEQ ID NOS: 1-12 and fragments thereof; and the polypeptide that specifically binds to human serum albumin comprises an amino acid selected from the group consisting of SEQ ID NOs: 22-34 and fragments thereof. In a particular embodiment, the polypeptide that specifically binds to human complement component C5 comprises the amino acid sequence of SEQ ID NO: 11 and the polypeptide that specifically binds to human serum albumin comprises the amino acid sequence of SEQ ID NO:26. In a particular embodiment, the fusion proteins described herein further comprise a peptide linker having an amino acid sequence of SEQ ID NO:102 or 103. In a particular embodiment, the fusion protein comprises a sequence that is at least 95% identical to a sequence selected from the group consisting of SEQ ID NOS: 96-101. In a particular embodiment, the fusion protein consists of a sequence selected from the group consisting of SEQ ID NOS: 96-101. In a particular embodiment, the fusion protein consists of a polypeptide sequence of SEQ ID NO:96. In a particular embodiment, the polypeptide that specifically binds to human complement component C5 comprises three complementarity determining regions, CDR1, CDR2 and CDR3, wherein CDR1 comprises any one of the amino acid sequences of SEQ ID NOS: 13-17, CDR2 comprises an amino acid sequences of SEQ ID NO:18 or 19, and CDR3 comprises an amino acid sequences of SEQ ID NO:20 or 21. In a particular embodiment, the polypeptide that specifically binds to human serum albumin comprises three complementarity determining regions, CDR1, CDR2 and CDR3, wherein CDR1 comprises any one of the amino acid sequences of SEQ ID NOS: 35-43, CDR2 comprises any one of the amino acid sequences of SEQ ID NOS: 44-51, and CDR3 comprises any one of the amino acid sequences of SEQ ID NOS: 52-63. In some embodiments, the antigen-binding domains described herein, can be engineered or further engineered to bind antigen in a pH-dependent manner, e.g., high affinity for antigen at high pH and a lower affinity for antigen binding at lower pH, or vice versa.

In one embodiment, the disclosure is directed to a pharmaceutical composition comprising a therapeutically effective amount of a fusion protein described herein and a pharmaceutically acceptable carrier. In a particular embodiment, the pharmaceutical compositions can contain an agent that degrades or inactivates hyaluronan, e.g., hyaluronidase or a recombinant hyaluronidase.

In one embodiment, the disclosure is directed to an isolated nucleic acid molecule comprising a nucleotide sequence encoding a fusion protein described herein. The nucleic acid molecule can be, for example, an expression vector. The disclosure is directed to host cells, (e.g., Chinese hamster ovary (CHO) cells, HEK293 cells, Pichia pastoris cells, mammalian cells, yeast cells, plant cells) and expression systems that comprise or utilize the nucleic acids that encode a fusion proteins described herein.

In one embodiment, the disclosure is directed to an engineered polypeptide that binds to human complement component C5, wherein the engineered polypeptide comprises an amino acid sequence selected from the group consisting of SEQ ID NOS: 1-12 and fragments thereof. In a particular embodiment, the engineered polypeptide comprises an amino acid sequence that is at least 90% identical (e.g., 90%, 91%, 92%, 93%, 94%, 95%, 96%, 97%, 98%, or 99% identical) to a sequence selected from the group consisting of SEQ ID NOS: 1-12. For example, in one embodiment, the engineered polypeptide comprises the amino acid sequence set forth in SEQ ID NO:1 or a sequence at least 90% identical thereto. In another embodiment, the engineered polypeptide comprises the amino acid sequence set forth in SEQ ID NO:2 or a sequence at least 90% identical thereto. In another embodiment, the engineered polypeptide comprises the amino acid sequence set forth in SEQ ID NO:3 or a sequence at least 90% identical thereto. In another embodiment, the engineered polypeptide comprises the amino acid sequence set forth in SEQ ID NO:4 or a sequence at least 90% identical thereto. In another embodiment, the engineered polypeptide comprises the amino acid sequence set forth in SEQ ID NO:5 or a sequence at least 90% identical thereto. In another embodiment, the engineered polypeptide comprises the amino acid sequence set forth in SEQ ID NO:6 or a sequence at least 90% identical thereto. In another embodiment, the engineered polypeptide comprises the amino acid sequence set forth in SEQ ID NO:7 or a sequence at least 90% identical thereto. In another embodiment, the engineered polypeptide comprises the amino acid sequence set forth in SEQ ID NO:8 or a sequence at least 90% identical thereto. In another embodiment, the engineered polypeptide comprises the amino acid sequence set forth in SEQ ID NO:9 or a sequence at least 90% identical thereto. In another embodiment, the engineered polypeptide comprises the amino acid sequence set forth in SEQ ID NO: 10 or a sequence at least 90% identical thereto. In another embodiment, the engineered polypeptide comprises the amino acid sequence set forth in SEQ ID NO:11 or a sequence at least 90% identical thereto. In another embodiment, the engineered polypeptide comprises the amino acid sequence set forth in SEQ ID NO: 12 or a sequence at least 90% identical thereto.

In another embodiment, an engineered polypeptide is provided that binds to human complement component C5, wherein the engineered polypeptide consists of an amino acid sequence selected from the group consisting of SEQ ID NOS: 1-12 and fragments thereof. For example, in one embodiment, the engineered polypeptide consists of the amino acid sequence set forth in SEQ ID NO:1. In another embodiment, the engineered polypeptide consists of the amino acid sequence set forth in SEQ ID NO:2. In another embodiment, the engineered polypeptide consists of the amino acid sequence set forth in SEQ ID NO:3. In another embodiment, the engineered polypeptide consists of the amino acid sequence set forth in SEQ ID NO:4. In another embodiment, the engineered polypeptide consists of the amino acid sequence set forth in SEQ ID NO:5. In another embodiment, the engineered polypeptide consists of the amino acid sequence set forth in SEQ ID NO:6. In another embodiment, the engineered polypeptide consists of the amino acid sequence set forth in SEQ ID NO:7. In another embodiment, the engineered polypeptide consists of the amino acid sequence set forth in SEQ ID NO:8. In another embodiment, the engineered polypeptide consists of the amino acid sequence set forth in SEQ ID NO:9. In another embodiment, the engineered polypeptide consists of the amino acid sequence set forth in SEQ ID NO: 10. In another embodiment, the engineered polypeptide consists of the amino acid sequence set forth in SEQ ID NO:11. In another embodiment, the engineered polypeptide consists of the amino acid sequence set forth in SEQ ID NO:12.

In one embodiment, the disclosure is directed to an engineered polypeptide that specifically binds to human serum albumin, wherein the polypeptide comprises and amino acid sequence selected from the group consisting of SEQ ID NOS: 22-34 and fragments thereof. In a particular embodiment, the engineered polypeptide comprises an amino acid sequence that is at least 90% identical (e.g., 90%, 91%, 92%, 93%, 94%, 95%, 96%, 97%, 98%, or 99% identical) to any one of the amino acid sequences of SEQ ID NOS: 22-34. For example, in one embodiment, the engineered polypeptide comprises the amino acid sequence set forth in SEQ ID NO:22 or a sequence at least 90% identical thereto. In another embodiment, the engineered polypeptide comprises the amino acid sequence set forth in SEQ ID NO:23 or a sequence at least 90% identical thereto. In another embodiment, the engineered polypeptide comprises the amino acid sequence set forth in SEQ ID NO:24 or a sequence at least 90% identical thereto. In another embodiment, the engineered polypeptide comprises the amino acid sequence set forth in SEQ ID NO:25 or a sequence at least 90% identical thereto. In another embodiment, the engineered polypeptide comprises the amino acid sequence set forth in SEQ ID NO:26 or a sequence at least 90% identical thereto. In another embodiment, the engineered polypeptide comprises the amino acid sequence set forth in SEQ ID NO:27 or a sequence at least 90% identical thereto. In another embodiment, the engineered polypeptide comprises the amino acid sequence set forth in SEQ ID NO:28 or a sequence at least 90% identical thereto. In another embodiment, the engineered polypeptide comprises the amino acid sequence set forth in SEQ ID NO:29 or a sequence at least 90% identical thereto. In another embodiment, the engineered polypeptide comprises the amino acid sequence set forth in SEQ ID NO:30 or a sequence at least 90% identical thereto. In another embodiment, the engineered polypeptide comprises the amino acid sequence set forth in SEQ ID NO:31 or a sequence at least 90% identical thereto. In another embodiment, the engineered polypeptide comprises the amino acid sequence set forth in SEQ ID NO:32 or a sequence at least 90% identical thereto. In another embodiment, the engineered polypeptide comprises the amino acid sequence set forth in SEQ ID NO:33 or a sequence at least 90% identical thereto. In another embodiment, the engineered polypeptide comprises the amino acid sequence set forth in SEQ ID NO:34 or a sequence at least 90% identical thereto.

In another embodiment, the engineered polypeptide that specifically binds to human serum albumin consists of an amino acid sequence selected from the group consisting of SEQ ID NOS: 22-34 and fragments thereof. For example, in one embodiment, the engineered polypeptide consists of the amino acid sequence set forth in SEQ ID NO:22. In another embodiment, the engineered polypeptide consists of the amino acid sequence set forth in SEQ ID NO:23. In another embodiment, the engineered polypeptide consists of the amino acid sequence set forth in SEQ ID NO:24. In another embodiment, the engineered polypeptide consists of the amino acid sequence set forth in SEQ ID NO:25. In another embodiment, the engineered polypeptide consists of the amino acid sequence set forth in SEQ ID NO:26. In another embodiment, the engineered polypeptide consists of the amino acid sequence set forth in SEQ ID NO:27. In another embodiment, the engineered polypeptide consists of the amino acid sequence set forth in SEQ ID NO:28. In another embodiment, the engineered polypeptide consists of the amino acid sequence set forth in SEQ ID NO:29. In another embodiment, the engineered polypeptide consists of the amino acid sequence set forth in SEQ ID NO:30. In another embodiment, the engineered polypeptide consists of the amino acid sequence set forth in SEQ ID NO:31. In another embodiment, the engineered polypeptide consists of the amino acid sequence set forth in SEQ ID NO:32. In another embodiment, the engineered polypeptide consists of the amino acid sequence set forth in SEQ ID NO:33. In another embodiment, the engineered polypeptide consists of the amino acid sequence set forth in SEQ ID NO:34.

In a particular embodiment, the engineered polypeptide that specifically binds to human serum albumin comprises three complementarity determining regions, CDR1, CDR2 and CDR3, wherein CDR1 comprises an amino acid sequence selected from the group consisting of SEQ ID NOs: 35-43, CDR2 comprises an amino acid sequence selected from the group consisting of SEQ ID NOs: 44-51, and CDR3 comprises an amino acid sequence selected from the group consisting of SEQ ID NOs: 52-63. In a particular embodiment, the polypeptide specifically binds to the same epitope on human serum albumin as Alb1.

In one embodiment, the disclosure is directed to a method for making a fusion protein described herein, comprising expressing in a host cell at least one nucleic acid molecule comprising a nucleotide sequence encoding the fusion protein.

In one embodiment, the disclosure is directed to a therapeutic kit comprising: (a) a container comprising a label; and (b) a composition comprising the fusion protein described herein; wherein the label indicates that the composition is to be administered to a patient having, or that is suspected of having, a complement-mediated disorder. The kit can optionally comprise an agent that degrades or inactivates hyaluronan, e.g., hyaluronidase or a recombinant hyaluronidase.

In one embodiment, the disclosure is directed to a method for treating a patient having a complement-mediated disorder, the method comprising administering to the patient a therapeutically effective amount of a fusion protein described herein. In a particular embodiment, the complement-mediated disorder is selected from the group consisting of: rheumatoid arthritis; lupus nephritis; asthma; ischemia-reperfusion injury;

atypical hemolytic uremic syndrome; dense deposit disease; paroxysmal nocturnal hemoglobinuria; macular degeneration; hemolysis, elevated liver enzymes, and low platelets (HELLP) syndrome; Guillain-Barré Syndrome; CHAPLE syndrome; myasthenia gravis; neuromyelitis optica; post-hematopoietic stem cell transplant thrombotic microangiopathy (post-HSCT-TMA); post-bone marrow transplant TMA (post-BMT TMA); Degos disease; Gaucher's disease; glomerulonephritis; thrombotic thrombocytopenia purpura (TTP); spontaneous fetal loss; Pauci-immune vasculitis; epidermolysis bullosa; recurrent fetal loss; multiple sclerosis (MS); traumatic brain injury; and injury resulting from myocardial infarction, cardiopulmonary bypass and hemodialysis.

### BRIEF DESCRIPTION OF THE DRAWINGS

FIGS. 1A and 1B show the results of a Complement Classical Pathway (CCP) hemolysis assay for anti-C5 VHH domains.

FIG. 2 shows the results of a C5a liberation assay for anti-C5 VHH domains.

FIGS. 3A-3D show the results of a CCP hemolysis assay for bispecific fusion proteins.

FIG. 4 shows the results of a Wieslab CCP assay for bispecific fusion proteins.

FIG. 5 shows the results of a C5a liberation assay for bispecific fusion proteins.

FIGS. 6A and 6B show the results of an LC-MS based quantitation assay demonstrating the pharmacokinetics of bispecific fusion proteins.

FIGS. 7A-7D show Biacore sensorgrams indicating the binding of FcRn at pH 6.0 in HBS-EP buffer to HSA saturated with no VHH domain (control, FIG. 7A), MSA21 (FIG. 7B), HAS040 (FIG. 7C) or HAS041 (FIG. 7D).

FIGS. 8A-8D show Biacore sensorgrams indicating the binding of albumin by the VHH domains HAS020, HAS040, HAS041 and HAS044 in competition with Alb1 VHH.

FIGS. 9A and 9B show the ability of various bi-specific fusion proteins to inhibit hemolysis.

FIG. 10 shows CRL0952 (SEQ ID NO:96) is functionally highly similar to CRL0500 in preventing hemolysis. CRL0500 is a bi-specific C5 and albumin binding fusion protein with a (G4S) 3 (SEQ ID NO:106) linker.

FIGS. 11A-11D show pH-dependent binding of histidine-substituted fusion proteins.

FIGS. 12A and 12B show pH-dependent binding of histidine-substituted fusion proteins.

### DETAILED DESCRIPTION

The disclosure provides engineered polypeptides that specifically bind to serum albumin or complement component C5, wherein the engineered polypeptides can be, for example, single-domain antibodies (sdAb's) or immunoglobulin (IgG) variable domains. In some embodiments, the engineered polypeptides do not significantly reduce or inhibit the binding of serum albumin to FcRn or do not significantly reduce the half-life of serum albumin. The disclosure also provides fusion proteins comprising engineered polypeptides, wherein the fusion proteins can be, for example, multivalent and multi-specific fusion proteins. The disclosure further provides nucleic acid molecules that encode engineered polypeptides or fusion proteins, and methods of making such engineered polypeptides or fusion proteins. The disclosure further provides pharmaceutical compositions that comprise engineered polypeptides or fusion proteins, and methods of treatment using such engineered polypeptides or fusion proteins.

Standard recombinant DNA methodologies are used to construct polynucleotides encoding the engineered polypeptides or fusion proteins of the disclosure, incorporate such polynucleotides into recombinant expression vectors, and introduce such vectors into host cells to produce the engineered polypeptides or fusion proteins of the disclosure. See e.g., Sambrook et al., 2001, MOLECULAR CLONING: A LABORATORY MANUAL (Cold Spring Harbor Laboratory Press, 3rd ed.). Unless specific definitions are provided, the nomenclature utilized in connection with, and the laboratory procedures and techniques of, analytical chemistry, synthetic organic chemistry, and medicinal and pharmaceutical chemistry described herein are those known and commonly used in the art. Similarly, conventional techniques can be used for chemical syntheses, chemical analyses, pharmaceutical preparation, formulation, delivery and treatment of patients.

### Definitions

As utilized in accordance with the present disclosure, the following terms, unless otherwise indicated, shall be understood to have the following meanings. Unless otherwise required by context, singular terms shall include pluralities and plural terms shall include the singular.

As used herein, the term “binding domain” refers to the portion of a protein or antibody which comprises the amino acid residues that interact with an antigen. Binding domains include, but are not limited to, antibodies (e.g., full length antibodies), as well as antigen-binding portions thereof. The binding domain confers on the binding agent its specificity and affinity for the antigen. The term also covers any protein having a binding domain which is homologous or largely homologous to an immunoglobulin-binding domain.

The term “antibody” as referred to herein includes whole antibodies and any antigen binding fragment (i.e., “antigen-binding portion”) or single chain version thereof. An “antibody” refers, in one preferred embodiment, to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding fragment” of an antibody (or simply “antibody fragment”), as used herein, refers to one or more fragments or portions of an antibody that retain the ability to specifically bind to an antigen. Such “fragments” are, for example between about 8 and about 1500 amino acids in length, suitably between about 8 and about 745 amino acids in length, suitably about 8 to about 300, for example about 8 to about 200 amino acids, or about 10 to about 50 or 100 amino acids in length. It has been shown that the antigen-binding function of an antibody can be performed by fragments of a full-length antibody. Examples of binding fragments encompassed within the term “antigen-binding fragment” of an antibody include (i) a Fab fragment, a monovalent fragment consisting of the V_{L}, V_{H}, CL and CH1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341: 544-546), which consists of a V_{H} domain; and (vi) an isolated complementarity determining region (CDR) or (vii) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (sFv); see e.g., Bird et al. (1988) Science 242: 423-426; and Huston et al. (1988) Proc. Natl. Acad. Sci. USA 85: 5879-5883). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies comprise variable and constant regions that utilize particular human germline immunoglobulin sequences are encoded by the germline genes, but include subsequent rearrangements and mutations which occur, for example, during antibody maturation. As known in the art (see, e.g., Lonberg (2005) Nature Biotech. 23 (9): 1117-1125), the variable region contains the antigen binding domain, which is encoded by various genes that rearrange to form an antibody specific for a foreign antigen. In addition to rearrangement, the variable region can be further modified by multiple single amino acid changes (referred to as somatic mutation or hypermutation) to increase the affinity of the antibody to the foreign antigen. The constant region will change in further response to an antigen (i.e., isotype switch). Therefore, the rearranged and somatically mutated nucleic acid molecules that encode the light chain and heavy chain immunoglobulin polypeptides in response to an antigen may not have sequence identity with the original nucleic acid molecules, but instead will be substantially identical or similar (i.e., have at least 80% identity).

The term “human antibody,” as used herein, refers to an immunoglobulin (Ig) that is used, for example, by the immune system to bind and neutralize pathogens. The term includes antibodies having variable and constant regions substantially corresponding to human germline Ig sequences. In some embodiments, human antibodies are produced in non-human mammals, including, but not limited to, rodents, such as mice and rats, and lagomorphs, such as rabbits. In other embodiments, human antibodies are produced in hybridoma cells. In still other embodiments, human antibodies are produced recombinantly. As used herein, human antibodies include all or a portion of an antibody, including, for example, heavy and light chains, variable regions, constant regions, proteolytic fragments, complementarity determining regions (CDRs), and other functional fragments.

As used herein, “biologically active fragment” refers to a portion of a molecule, e.g., a gene, coding sequence, mRNA, polypeptide or protein, which has a desired length or biological function. A biologically active fragment of a protein, for example, can be a fragment of the full-length protein that retains one or more biological activities of the protein. A biologically active fragment of an mRNA, for example, can be a fragment that, when translated, expresses a biologically active protein fragment. A biologically active mRNA fragment, furthermore, can comprise shortened versions of non-coding sequences, e.g., regulatory sequences, UTRs, etc. In general, a fragment of an enzyme or signaling molecule can be, for example, that portion(s) of the molecule that retains its signaling or enzymatic activity. A fragment of a gene or coding sequence, for example, can be that portion of the gene or coding sequence that produces an expression product fragment. A fragment does not necessarily have to be defined functionally, as it can also refer to a portion of a molecule that is not the whole molecule, but has some desired characteristic or length (e.g., restriction fragments, proteolytic fragment of a protein, amplification fragments, etc.).

Ordinary or conventional mammalian antibodies comprise a tetramer, which is typically composed of two identical pairs of polypeptide chains, each pair having one full-length “light” chain (typically having a molecular weight of about 25 kDa) and one full-length “heavy” chain (typically having a molecular weight of about 50-70 kDa). The terms “heavy chain” and “light chain,” as used herein, refer to any Ig polypeptide having sufficient variable domain sequence to confer specificity for a target antigen. The N-terminal portion of each light and heavy chain typically includes a variable domain of about 100 to 110 or more amino acids that typically is responsible for antigen recognition. The C-terminal portion of each chain typically defines a constant domain responsible for effector function. Thus, in a naturally occurring antibody, a full-length heavy chain Ig polypeptide includes a variable domain (V_{H} or VH) and three constant domains (C_{H1} or CH1, C_{H2} or CH2, and C_{H3} or CH3), wherein the V_{H} domain is at the N-terminus of the polypeptide and the C_{H3} domain is at the C-terminus, and a full-length light chain Ig polypeptide includes a variable domain (V_{L} or VL) and a constant domain (C_{L} or CL), wherein the V_{L} domain is at the N-terminus of the polypeptide and the C_{L} domain is at the C-terminus.

Within full-length light and heavy chains, the variable and constant domains typically are joined by a “J” region of about 12 or more amino acids, with the heavy chain also including a “D” region of about 10 more amino acids. The variable regions of each light/heavy chain pair typically form an antigen-binding site. The variable domains of naturally occurring antibodies typically exhibit the same general structure of relatively conserved framework regions (FR) joined by three hypervariable regions called CDRs. The CDRs from the two chains of each pair typically are aligned by the framework regions, which enables binding to a specific epitope. From the N-terminus to the C-terminus, both light and heavy chain variable domains typically comprise the domains FR1, CDR1, FR2, CDR2, FR3, CDR3 and FR4.

The terms “substantially pure” or “substantially purified,” as used herein, refer to a compound or species that is the predominant species present in a composition (i.e., on a molar basis it is more abundant than any other individual species in the composition). A substantially purified fraction, for example, can be a composition wherein the predominant species comprises at least about 50% (on a molar basis) of all macromolecular species present. A substantially pure composition, for example, can comprise a predominant species that represents more than about 80%, 85%, 90%, 95% or 99% of all macromolar species present in the composition. In other embodiments, the predominant species can be purified to substantial homogeneity (contaminant species cannot be detected in the composition by conventional detection methods) wherein the composition consists essentially of a single macromolecular species.
The terms “antigen” or “antigen target,” as used herein, refer to a molecule or a portion of a molecule that is capable of being bound to by an antibody, one or more Ig binding domain, or other immunological binding moiety, including, for example, the engineered polypeptides or fusion proteins disclosed herein. An antigen is capable of being used in an animal to produce antibodies capable of binding to an epitope of that antigen. An antigen may have one or more epitopes.

The term “epitope” or “antigenic determinant” refers to a site on an antigen to which an immunoglobulin or antibody specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from the antigen are tested for reactivity with the given antibody. Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)).

The terms “activity,” “biological activity,” or “biological property,” as used in reference to the engineered polypeptides or fusion proteins of the disclosure, include, but are not limited to, epitope affinity and specificity, ability to antagonize the activity of an antigen target, the in vivo stability of the engineered polypeptides or fusion proteins of the disclosure, and the immunogenic properties of the engineered polypeptides or fusion proteins of the disclosure. Other identifiable biological properties include, for example, cross-reactivity (e.g., with non-human homologs of the antigen target, or with other antigen targets or tissues, generally), and ability to preserve high expression levels of protein in mammalian cells.

An antibody, immunoglobulin, or immunologically functional immunoglobulin fragment, or the engineered polypeptides or fusion proteins disclosed herein, are said to “specifically” bind an antigen when the molecule preferentially recognizes its antigen target in a complex mixture of proteins and/or macromolecules. The term “specifically binds,” as used herein, refers to the ability of an antibody, immunoglobulin, or immunologically functional immunoglobulin fragment, or an engineered polypeptide or fusion protein of the disclosure, to bind to an antigen containing an epitope with an K_{D} of at least about 10⁻⁶ M, 10⁻⁷ M, 10⁻⁸ M, 10⁻⁹ M, 10⁻¹⁰ M, 10⁻¹¹ M, 10⁻¹² M, or more, and/or to bind to an epitope with an affinity that is at least two-fold greater than its affinity for a nonspecific antigen.

The term “K_{D},” as used herein, refers to the dissociation constant of the interaction between an antibody, immunoglobulin, or immunologically functional immunoglobulin fragment, or an engineered polypeptide or fusion protein disclosed herein, and an antigen target. When an engineered polypeptide or fusion protein of the disclosure comprises a monovalent Ig sequence, the monovalent Ig sequence preferably binds to a desired antigen, for example, with a K_{D} of 10⁻⁵ to 10⁻¹² M or less, or 10⁻⁷ to 10⁻¹² M or less, or 10⁻³ to 10⁻¹² M, and/or with a binding affinity of at least 10⁷ M⁻¹, at least 10⁸ M⁻¹, at least 10⁹ M⁻¹, or at least 10¹² M⁻¹. A K_{D} value greater than 10⁻⁴ M is generally considered to indicate non-specific binding. In some embodiments, a monovalent Ig sequence of an engineered polypeptide or fusion protein of the disclosure binds to a desired antigen with an affinity less than 500 mM, less than 200 nM, less than 10 nM, or less than 500 pM.

A K_{D} can be determined by methods known in the art, including, for example, surface plasmon resonance (SPR). Generally, SPR analysis measures real-time binding interactions between a ligand (a target antigen on a biosensor matrix) and an analyte using, for example, the BIAcore system (Pharmacia Biosensor; Piscataway, NJ). SPR analysis can also be performed by immobilizing an analyte and presenting the ligand.

Specific binding of an engineered polypeptide or fusion protein of the disclosure to an antigen or antigenic determinant can also be determined in any suitable manner known in the art, including, for example, Scatchard analysis and/or competitive binding assays, such as radioimmunoassays (RIA), enzyme immunoassays (EIA) and sandwich competition assays.

The term “bispecific” refers to a fusion protein of the disclosure that is capable of binding two antigens. The term “multivalent fusion protein” means a fusion protein comprising two or more antigen binding sites.

The term “multi-specific fusion protein” refers to a fusion protein of the disclosure that is capable of binding two or more related or unrelated targets.

The term “fused to” as used herein refers to a polypeptide made by combining more than one sequence, typically by cloning one sequence, e.g., a coding sequence, into an expression vector in frame with one or more second coding sequence(s) such that the two (or more) coding sequences are transcribed and translated into a single continuous polypeptide. In addition to being made by recombinant technology, parts of a polypeptide can be “fused to” each other by means of chemical reaction, or other means known in the art for making custom polypeptides.

The term “vector,” as used herein, refers to any molecule (e.g., nucleic acid, plasmid or virus) that is used to transfer coding information to an expression system (e.g., a host cell or in vitro expression system). One type of vector is a “plasmid,” which refers to a circular double-stranded DNA (dsDNA) molecule into which additional DNA segments can be inserted. Another type of vector is a viral vector, wherein additional DNA segments can be inserted into a viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell and thereby are replicated along with the host genome. In addition, certain vectors are capable of directing the expression of coding sequences to which they are operatively linked. Such vectors are referred to herein as “expression vectors.”

The term “operably linked,” as used herein, refers to an arrangement of flanking sequences wherein the flanking sequences are configured or assembled to perform a desired function. Thus, a flanking sequence operably linked to a coding sequence may be capable of effecting the replication, transcription, and/or translation of the coding sequence. A coding sequence is operably linked to a promoter, for example, where the promoter is capable of directing transcription of that coding sequence. A flanking sequence need not be contiguous with the coding sequence to be considered operably linked, so long as it functions correctly.

The term “host cell,” as used herein, refers to a cell into which an expression vector has been introduced. A host cell is intended to refer not only to the particular subject cell, but also to the progeny of such a cell. Because certain modifications may occur in succeeding generations due to either mutation or environmental influences, such progeny may not be, in fact, identical to the parent cell, but such cells are still included within the scope of the term “host cell” as used herein. A wide variety of host cell expression systems can be used to express the engineered polypeptides or fusion proteins of the disclosure, including bacterial, yeast, baculoviral, and mammalian expression systems (as well as phage display expression systems).

The term “naturally occurring,” as used herein and applied to a particular molecule, refers to a molecule that is found in nature and has not been manipulated by man. Similarly, the term “non-naturally occurring,” as used herein, refers to a molecule that is not found in nature or that has been modified or artificially synthesized.

The term “engineered,” as used herein and applied to a particular molecule, such as, for example, a polypeptide, that has been modified or manipulated, such as by mutation, truncation, deletion, substitution, addition, conjugation or by otherwise changing the primary sequence, chemical or three-dimensional structure, chemical signature, folding behavior, glycosylation state, or any other attribute of the molecule, such that the molecule differs from its naturally occurring counterpart.

The term “patient” as used herein includes human and animal subjects.

A “disorder” is any condition that would benefit from treatment using the engineered polypeptides or fusion proteins of the disclosure. “Disorder” and “condition” are used interchangeably herein.

A “complement-mediated disorder” as used herein refers to a disorder caused, directly or indirectly, by mis-regulation of the complement pathway, e.g., activation or suppression of the complement pathway, or a disorder that is mediated, directly or indirectly, by one or more components of the complement pathway, or a product generated by the complement pathway. The term also refers to a disorder that is exacerbated by one or more components of the complement pathway, or a product generated by the complement pathway.

The terms “treatment” or “treat,” as used herein, refer to both therapeutic treatment and prophylactic or preventative measures. Those in need of treatment include those having the disorder as well as those at risk of having the disorder or those in which the disorder is to be prevented.

As used herein, a “therapeutically effective” amount of, for example, a fusion protein or engineered polypeptide described herein, is an amount that, when administered, results in a decrease in severity of disease symptoms (e.g., a decrease in symptoms of disorders associated with a complement-mediated disorder, an increase in frequency and duration of disease symptom free periods, or a prevention of impairment or disability due to the disease affliction. In certain embodiments, a therapeutically effective amount of a therapeutic agent described herein can include an amount (or various amounts in the case of multiple administrations) that reduces hemolysis, or improves symptoms of a complement-mediated disorder.

The terms “pharmaceutical composition” or “therapeutic composition,” as used herein, refer to a compound or composition capable of inducing a desired therapeutic effect when administered to a patient.

The term “pharmaceutically acceptable carrier” or “physiologically acceptable carrier,” as used herein, refers to one or more formulation materials suitable for accomplishing or enhancing the delivery of the engineered polypeptides or fusion proteins of the disclosure.

The term “therapeutically effective amount,” as used in reference to a pharmaceutical composition comprising one or more engineered polypeptides or fusion proteins of the disclosure, refers to an amount or dosage sufficient to produce a desired therapeutic result. More specifically, a therapeutically effective amount is an amount of one or more engineered polypeptides or fusion proteins of the disclosure sufficient to inhibit, for some period of time, one or more of the clinically defined pathological processes associated with the condition being treated, e.g., a complement-mediated disorder. The therapeutically effective amount may vary depending on the specific engineered polypeptide or fusion protein that is being used, and depends on a variety of factors and conditions related to the patient being treated and the severity of the disorder.

### Complement System

The complement system acts in conjunction with other immunological systems of the body to defend against intrusion of cellular and viral pathogens. There are at least 25 complement proteins, which are a complex collection of plasma proteins and membrane cofactors. The plasma proteins make up about 10% of the globulins in vertebrate serum. Complement components achieve their immune defensive functions by interacting in a series of intricate but precise enzymatic cleavage and membrane binding events. The resulting complement cascade leads to the production of products with opsonic, immunoregulatory and lytic functions.

The complement cascade can progress via the classical pathway (CP), the lectin pathway or the alternative pathway (AP). The lectin pathway is typically initiated with binding of mannose-binding lectin (MBL) to high mannose substrates. The AP can be antibody independent and initiated by certain molecules on pathogen surfaces. The CP is typically initiated by antibody recognition of, and binding to, an antigenic site on a target cell. These pathways converge at the C3 convertase-where complement component C3 is cleaved by an active protease to yield C3a and C3b.

Spontaneous hydrolysis of complement component C3, which is abundant in the plasma fraction of blood, can also lead to AP C3 convertase initiation. This process, known as “tickover,” occurs through the spontaneous cleavage of a thioester bond in C3 to form C3i or C3(H₂0). Tickover is facilitated by the presence of surfaces that support the binding of activated C3 and/or have neutral or positive charge characteristics (e.g., bacterial cell surfaces). Formation of C3(H₂0) allows for the binding of plasma protein Factor B, which in turn allows Factor D to cleave Factor B into Ba and Bb. The Bb fragment remains bound to C3 to form a complex containing C3(H₂0)Bb—the “fluid-phase” or “initiation” C3 convertase. Although only produced in small amounts, the fluid-phase C3 convertase can cleave multiple C3 proteins into C3a and C3b and results in the generation of C3b and its subsequent covalent binding to a surface (e.g., a bacterial surface). Factor B bound to the surface-bound C3b is cleaved by Factor D to form the surface-bound AP C3 convertase complex containing C3b,Bb.

The AP C5 convertase ((C3b)₂,Bb) is formed upon addition of a second C3b monomer to the AP C3 convertase. The role of the second C3b molecule is to bind C5 and present it for cleavage by Bb. The AP C3 and C5 convertases are stabilized by the addition of the trimeric protein properdin. Properdin binding, however, is not required to form a functioning alternative pathway C3 or C5 convertase.

The CP C3 convertase is formed upon interaction of complement component C1, which is a complex of C1q, C1r and C1s, with an antibody that is bound to a target antigen (e.g., a microbial antigen). The binding of the C1q portion of C1 to the antibody-antigen complex causes a conformational change in C1 that activates C1r. Active C1r then cleaves the C1-associated C1s to generate an active serine protease. Active C1s cleaves complement component C4 into C4b and C4a. Like C3b, the newly generated C4b fragment contains a highly reactive thiol that readily forms amide or ester bonds with suitable molecules on a target surface (e.g., a microbial cell surface). C1s also cleaves complement component C2 into C2b and C2a. The complex formed by C4b and C2a is the CP C3 convertase, which is capable of processing C3 into C3a and C3b. The CP C5 convertase (C4b, C2a, C3b) is formed upon addition of a C3b monomer to the CP C3 convertase.

In addition to its role in C3 and C5 convertases, C3b also functions as an opsonin through its interaction with complement receptors present on the surfaces of antigen-presenting cells such as macrophages and dendritic cells. The opsonic function of C3b is generally considered one of the most important anti-infective functions of the complement system. Patients with genetic lesions that block C3b function are prone to infection by a broad variety of pathogenic organisms, while patients with lesions later in the complement cascade sequence, i.e., patients with lesions that block C5 functions, are found to be more prone only to Neisseria infection, and then only somewhat more prone.

The AP and CP C5 convertases cleave C5 into C5a and C5b. Cleavage of C5 releases C5a, a potent anaphylatoxin and chemotactic factor, and C5b, which allows for the formation of the lytic terminal complement complex, C5b-9. C5b combines with C6, C7 and C8 to form the C5b-8 complex at the surface of the target cell. Upon binding of several C9 molecules, the membrane attack complex (MAC, C5b-9, terminal complement complex (“TCC”)) is formed. When sufficient numbers of MACs insert into target cell membranes, the openings they create (MAC pores) mediate rapid osmotic lysis of the target cells.

While a properly functioning complement system provides a robust defense against infecting microbes, inappropriate regulation or activation of the complement pathways has been implicated in the pathogenesis of a variety of disorders including, e.g., rheumatoid arthritis (RA); lupus nephritis; asthma; ischemia-reperfusion injury; atypical hemolytic uremic syndrome (aHUS); dense deposit disease (DDD); paroxysmal nocturnal hemoglobinuria (PNH); macular degeneration (e.g., age-related macular degeneration (AMD)); hemolysis, elevated liver enzymes and low platelets (HELLP) syndrome; Guillain-Barré Syndrome (GBS); protein-losing enteropathy (e.g., CHAPLE syndrome); myasthenia gravis (MG); neuromyelitis optica (NMO); post-hematopoietic stem cell transplant thrombotic microangiopathy (post-HSCT-TMA); post-bone marrow transplant TMA (post-BMT TMA); Degos disease; Gaucher's disease; glomerulonephritis; thrombotic thrombocytopenia purpura (TTP); spontaneous fetal loss; Pauci-immune vasculitis; epidermolysis bullosa; recurrent fetal loss; multiple sclerosis (MS); traumatic brain injury; and injury resulting from myocardial infarction, cardiopulmonary bypass and hemodialysis (Holers, V., Immunol. Rev., 223: 300-16, 2008). The down-regulation of complement activation has been demonstrated to be effective in treating several disease indications in a variety of animal models (Rother, R. et al., Nat. Biotechnol., 25: 1256-64, 2007; Wang, Y. et al., Proc. Natl. Acad. Sci. USA, 93: 8563-8, 1996; Wang, Y. et al., Proc. Natl. Acad. Sci. USA, 92: 8955-9, 1995; Rinder, C. et al., J. Clin. Invest., 96: 1564-72, 1995; Kroshus, T. et al., Transplantation, 60: 1194-202, 1995; Homeister, J. et al., J. Immunol., 150: 1055-64, 1993; Weisman, H. et al., Science, 249: 146-51, 1990; Amsterdam, E. et al., Am. J. Physiol., 268: H448-57, 1995; and Rabinovici, R. et al., J. Immunol., 149: 1744-50, 1992).

### Human Serum Albumin and Neonatal Fc Receptor

Polypeptides that can bind to human serum albumin (HSA) to increase the half-life of therapeutically relevant proteins have been described (WO 91/01743, WO 01/45746 and WO 02/076489). The described peptide moieties, however, are of bacterial or synthetic origin, which is not preferred for use in therapeutics in humans. WO 04/041865 describes single-domain antibodies (sdAb's or Nanobodies®) directed against serum albumin (and in particular against HSA) that can be linked to other proteins (such as one or more other sdAb's directed against a desired target) to increase the half-life of the protein.

The neonatal Fc receptor (FcRn), also termed “Brambell receptor,” is involved in prolonging the lifespan of albumin in circulation (Chaudhury, C. et al., J. Exp. Med., 3: 315-22, 2003). FcRn is an integral membrane glycoprotein consisting of a soluble light chain consisting of β2-microglobulin (β2m), non-covalently bound to a 43 kDa α chain with three extracellular domains, a transmembrane region and a cytoplasmic tail of about 50 amino acids. The cytoplasmic tail contains a dinucleotide motif endocytosis signal implicated in the internalization of the receptor. The α chain is a member of the non-classical MHC I family of proteins. The β2m association with the α chain is critical for correct folding of FcRn and exiting the endoplasmic reticulum for routing to endosomes and the cell surface.

The overall structure of FcRn is similar to that of class I molecules. The α-1 and α-2 regions resemble a platform composed of eight antiparallel strands forming a single β-sheet topped by two antiparallel α-helices very closely resembling the peptide cleft in MHC I molecules. Owing to an overall repositioning of the α-1 helix and bending of the C-terminal portion of the α-2 helix due to a break in the helix introduced by the presence of Pro162, the FcRn helices are close in proximity, occluding peptide binding. The side chain of Arg164 of FcRn also occludes the potential interaction of the peptide N-terminus with the MHC pocket. Further, salt bridge and hydrophobic interaction between the α-1 and α-2 helices may also contribute to the groove closure. FcRn therefore, does not participate in antigen presentation and the peptide cleft is empty.

FcRn binds and transports IgG across the placental syncytiotrophoblast from maternal circulation to fetal circulation and protects IgG from degradation in adults. In addition to homeostasis, FcRn controls transcytosis of IgG in tissues. FcRn is localized in epithelial cells, endothelial cells, and hepatocytes.

HSA binds FcRn to form a tri-molecular complex with IgG. Both albumin and IgG bind non-cooperatively to distinct sites on FcRn. Binding of human FcRn to Sepharose-HSA and Sepharose-hIgG is pH dependent, being maximal at pH 5 and undetectable at pH 7 through pH 8. The observation that FcRn binds albumin in the same pH-dependent fashion as it binds IgG suggests that the mechanism by which albumin interacts with FcRn and thus is protected from degradation is identical to that of IgG, and mediated via a similarly pH-sensitive interaction with FcRn. Using surface plasmon resonance to measure the capacity of individual HSA domains to bind immobilized soluble hFcRn, FcRn and albumin have been shown to interact via the D-III domain of albumin in a pH-dependent manner, on a site distinct from the IgG binding site (Chaudhury, C. et al., Biochemistry, 45: 4983-90, 2006).

### Engineered Polypeptides Specifically Bind Complement C5 or Serum Albumin

Described herein are engineered polypeptides comprising Ig sequences, e.g., Ig variable domain sequences, that can bind or otherwise associate with complement component C5 or serum albumin. Engineered polypeptides described herein can specifically bind serum albumin in such a way that, when the engineered polypeptide is bound to or otherwise associated with a serum albumin molecule, the binding of the serum albumin molecule to FcRn is not significantly reduced or inhibited as compared to the binding of the serum albumin molecule to FcRn when the polypeptide is not bound thereto. In this embodiment, “not significantly reduced or inhibited” means that the binding affinity for serum albumin to FcRn (as measured using a suitable assay, such as, for example, SPR) is not reduced by more than 50%, or by more than 30%, or by more than 10%, or by more than 5%, or not reduced at all. In this embodiment, “not significantly reduced or inhibited” also means that the half-life of the serum albumin molecule is not significantly reduced. In particular, the engineered polypeptides can to amino acid residues on serum albumin that are not involved in binding of serum albumin to FcRn. More particularly, engineered polypeptides can bind to amino acid residues or sequences of serum albumin that do not form part of domain III of serum albumin, e.g., engineered polypeptides that are capable of binding to amino acid residues or sequences of serum albumin that form part of domain I and/or domain II.

In some embodiments, the engineered polypeptides are sdAbs or suitable for use as sdAbs, and as such may be a heavy chain variable domain sequence or a light chain variable domain sequence, and in certain embodiments, are heavy chain variable domain sequences of a heavy chain antibody. In cases where the engineered polypeptides are single domain, heavy chain variable domain sequences from a heavy chain antibody, such sequences may be referred to as VHH or V_{H}H antibodies, VHH or V_{H}H antibody fragments, or VHH or V_{H}H domains.

A “heavy chain antibody” refers to an antibody that consists of two heavy chains and lacks the two light chains found in conventional antibodies. Camelids (members of the biological family Camelidae, the only currently living family in the suborder Tylopoda; extant camelids include dromedary camels, Bactrian camels, wild or feral camels, llamas, alpacas, vicuñas and guanacos) are the only mammals with single chain VHH antibodies. About 50% of the antibodies in camelids are heavy chain antibodies with the other 50% being of the ordinary or conventional mammalian heavy/light chain antibody type.

“VHH domain” refers to variable domains present in naturally occurring heavy chain antibodies to distinguish them from the heavy chain variable domains that are present in conventional four chain antibodies (referred to herein as “VH domains”) and from the light chain variable domains that present in conventional four chain antibodies (referred to herein as “VL domains”).

VHH domains have a number of unique structural characteristics and functional properties that make isolated VHH domains (as well as sdAbs, which are based on VHH domains and share these structural characteristics and functional properties with the naturally occurring VHH domains) and proteins containing the VHH domains highly advantageous for use as functional antigen binding domains or proteins. For example, VHH domains, which bind to an antigen without the presence of a VL, and sdAbs can function as a single, relatively small, functional antigen binding structural unit, domain or protein. The small size of these molecules distinguishes VHH domains from the VH and VL domains of conventional four-chain antibodies. The use of VHH domains and sdAbs as single antigen-binding proteins or as antigen-binding domains (e.g., as part of a larger protein or polypeptide) offers a number of significant advantages over the use of conventional VH and VL domains, as well as scFv or conventional antibody fragments (such as Fab or F(ab′)₂ fragments). Only a single domain is required to bind an antigen with high affinity and with high selectivity, for example, so that there is no need to have two separate domains present, nor to assure that these two domains are present in a particular spatial conformation and configuration (e.g., through the use of specific linkers, as with an scFv). VHH domains and sdAbs can also be expressed from a single gene and require no post-translational folding or modifications. VHH domains and sdAbs can easily be engineered into multivalent and multi-specific formats. VHH domains and sdAbs are also highly soluble and do not have a tendency to aggregate (Ward, E. et al., Nature, 341: 544-6, 1989), and they are highly stable to heat, pH, proteases and other denaturing agents or conditions (Ewert, S. et al., Biochemistry, 41: 3628-36, 2002). VHH domains and sdAbs are relatively easy and cheap to prepare, even on a scale required for production. For example, VHH domains, sdAbs, and polypeptides containing VHH domains or sdAbs can be produced using microbial fermentation using methods known in the art and do not require the use of mammalian expression systems, as with, for example, conventional antibody fragments. VHH domains and sdAbs are relatively small (approximately 15 kDa, or 10 times smaller than a conventional IgG) compared to conventional four-chain antibodies and antigen-binding fragments thereof, and therefore show higher penetration into tissues (including but not limited to solid tumors and other dense tissues) than conventional four-chain antibodies and antigen-binding fragments thereof. VHH domains and sdAbs can show so-called “cavity-binding” properties (due to, for example, their extended CDR3 loop) and can access targets and epitopes not accessible to conventional four-chain antibodies and antigen-binding fragments thereof. It has been shown, for example, that VHH domains and sdAbs can inhibit enzymes (WO 97/49805; Transue, T. et al., Proteins, 32: 515-22, 1998; Lauwereys, M. et al., EMBO J., 17: 3512-20, 1998).

The term “single-domain antibody,” or “sdAb,” as used herein, is an antibody or fragment thereof consisting of a single monomeric variable antibody domain. It is not limited to a specific biological source or to a specific method of preparation. A sdAb can be obtained, for example, by (1) isolating the VHH domain of a naturally occurring heavy chain antibody; (2) expressing a nucleotide sequence encoding a naturally occurring VHH domain; (3) “humanization” of a naturally occurring VHH domain or by expression of a nucleic acid encoding such humanized VHH domain; (4) “camelization” of a naturally occurring VH domain from any animal species, in particular a species of mammal, such as from a human being, or by expression of a nucleic acid encoding such a camelized VH domain; (5) “camelization” of a “domain antibody” (“Dab”) or by expression of a nucleic acid encoding such a camelized VH domain; (6) using synthetic or semi-synthetic techniques for preparing engineered polypeptides or fusion proteins; (7) preparing a nucleic acid encoding a sdAb using techniques for nucleic acid synthesis, followed by expression of the nucleic acid thus obtained; and/or (8) any combination of the above.

The engineered polypeptides or fusion proteins described herein can comprise, for example, amino acid sequences of naturally occurring VHH domains that have been “humanized,” e.g., by replacing one or more amino acid residues in the amino acid sequence of the naturally occurring VHH sequence by one or more of the amino acid residues that occur at the corresponding positions in a VH domain from a human being.

The engineered polypeptides or fusion proteins described herein can comprise, for example, amino acid sequences of naturally occurring VH domains that have been “camelized,” i.e., by replacing one or more amino acid residues in the amino acid sequence of a naturally occurring VH domain with one or more of the amino acid residues that occur at the corresponding positions in a VHH domain of, for example, a camelid antibody. This can be performed in a manner known in the art. Such camelization may preferentially occur at amino acid positions that are present at the VH-VL interface and at the so-called “Camelidae hallmark residues” (WO 94/04678). The VH domain or sequence that is used as a parental sequence or starting material for generating or designing the camelized sequence can be, for example, a VH sequence from a mammal, and in certain embodiments, the VH sequence of a human. It should be noted, however, that such camelized sequences can be obtained in any suitable manner known in the art and thus are not strictly limited to polypeptides that have been obtained using a polypeptide that comprises a naturally occurring parental VH domain.

Both “humanization” and “camelization” can be performed by providing a nucleotide sequence that encodes a naturally occurring VHH domain or V_{H} domain, respectively, and then changing, in a manner known to those skilled in the art, one or more codons in the nucleotide sequence such that the new nucleotide sequence encodes a humanized or camelized sequence, respectively. Also, based on the amino acid sequence or nucleotide sequence of a naturally occurring VHH domain or VH domain, a nucleotide sequence encoding a desired humanized or camelized sequence can be designed and synthesized de novo using techniques for nucleic acid synthesis known in the art, after which the nucleotide sequence thus obtained can be expressed in a manner known in the art.

In some embodiments, the disclosure provides an engineered polypeptide that specifically binds to the same epitope on human C5 as eculizumab, or that binds to an epitope on C5 that prevents cleavage of C5 into C5a and C5b. In some embodiments, the disclosure provides an engineered polypeptide that specifically binds to human complement component C5, wherein the polypeptide comprises any one of the amino acid sequences of SEQ ID NOs: 1-12 or a fragment thereof. In other embodiments, the disclosure provides an engineered polypeptide that specifically binds to human complement component C5, wherein the polypeptide comprises an amino acid sequence that is at least 90% identical to any one of the amino acid sequences of SEQ ID NOs: 1-12. In other embodiments, the disclosure provides an engineered polypeptide that specifically binds to human complement component C5, wherein the polypeptide comprises an amino acid sequence that is at least 95% identical, at least 96% identical, at least 97% identical, at least 98% identical, or at least 99% identical to any one of the amino acid sequences of SEQ ID NOs: 1-12. For example, in one embodiment, the engineered polypeptide comprises the amino acid sequence set forth in SEQ ID NO:1 or a sequence at least 90% identical thereto. In another embodiment, the engineered polypeptide comprises the amino acid sequence set forth in SEQ ID NO:2 or a sequence at least 90% identical thereto. In another embodiment, the engineered polypeptide comprises the amino acid sequence set forth in SEQ ID NO:3 or a sequence at least 90% identical thereto. In another embodiment, the engineered polypeptide comprises the amino acid sequence set forth in SEQ ID NO:4 or a sequence at least 90% identical thereto. In another embodiment, the engineered polypeptide comprises the amino acid sequence set forth in SEQ ID NO:5 or a sequence at least 90% identical thereto. In another embodiment, the engineered polypeptide comprises the amino acid sequence set forth in SEQ ID NO:6 or a sequence at least 90% identical thereto. In another embodiment, the engineered polypeptide comprises the amino acid sequence set forth in SEQ ID NO:7 or a sequence at least 90% identical thereto. In another embodiment, the engineered polypeptide comprises the amino acid sequence set forth in SEQ ID NO:8 or a sequence at least 90% identical thereto. In another embodiment, the engineered polypeptide comprises the amino acid sequence set forth in SEQ ID NO:9 or a sequence at least 90% identical thereto. In another embodiment, the engineered polypeptide comprises the amino acid sequence set forth in SEQ ID NO:10 or a sequence at least 90% identical thereto. In another embodiment, the engineered polypeptide comprises the amino acid sequence set forth in SEQ ID NO:11 or a sequence at least 90% identical thereto. In another embodiment, the engineered polypeptide comprises the amino acid sequence set forth in SEQ ID NO: 12 or a sequence at least 90% identical thereto.

In another embodiment, an engineered polypeptide is provided that binds to human complement component C5, wherein the engineered polypeptide consists of an amino acid sequence selected from the group consisting of SEQ ID NOS: 1-12 and fragments thereof. For example, in one embodiment, the engineered polypeptide consists of the amino acid sequence set forth in SEQ ID NO:1. In another embodiment, the engineered polypeptide consists of the amino acid sequence set forth in SEQ ID NO:2. In another embodiment, the engineered polypeptide consists of the amino acid sequence set forth in SEQ ID NO:3. In another embodiment, the engineered polypeptide consists of the amino acid sequence set forth in SEQ ID NO:4. In another embodiment, the engineered polypeptide consists of the amino acid sequence set forth in SEQ ID NO:5. In another embodiment, the engineered polypeptide consists of the amino acid sequence set forth in SEQ ID NO:6. In another embodiment, the engineered polypeptide consists of the amino acid sequence set forth in SEQ ID NO:7. In another embodiment, the engineered polypeptide consists of the amino acid sequence set forth in SEQ ID NO:8. In another embodiment, the engineered polypeptide consists of the amino acid sequence set forth in SEQ ID NO:9. In another embodiment, the engineered polypeptide consists of the amino acid sequence set forth in SEQ ID NO: 10. In another embodiment, the engineered polypeptide consists of the amino acid sequence set forth in SEQ ID NO:11. In another embodiment, the engineered polypeptide consists of the amino acid sequence set forth in SEQ ID NO:12.

In another embodiment, the disclosure provides an engineered polypeptide that specifically binds to human complement component C5, wherein the polypeptide comprises three complementarity determining regions, CDR1, CDR2 and CDR3, wherein CDR1 comprises any one of the amino acid sequences of SEQ ID NOs: 13-17 or a sequence that is at least 90% identical to SEQ ID NOs: 13-17; CDR2 comprises an amino acid sequence of SEQ ID NOs: 18 or 19 or a sequence that is at least 90% identical to SEQ ID NOs: 18 or 19; and CDR3 comprises an amino acid sequence of SEQ ID NOs: 20 or 21 or a sequence that is at least 90% identical to SEQ ID NOs: 20 or 21.

In other embodiments, the disclosure provides an engineered polypeptide that specifically binds to human serum albumin, wherein the polypeptide comprises any one of the amino acid sequences of SEQ ID NOs: 22-34, or a fragment thereof. In other embodiments, the disclosure provides an engineered polypeptide that specifically binds to human serum albumin, wherein the polypeptide comprises an amino acid sequence that is at least 90% identical to any one of the amino acid sequences of SEQ ID NOs: 22-34. In other embodiments, the disclosure provides an engineered polypeptide that specifically binds to human serum albumin, wherein the polypeptide comprises an amino acid sequence that is at least 95% identical, at least 96% identical, at least 97% identical, at least 98% identical, or at least 99% identical to any one of the amino acid sequences of SEQ ID NOs: 22-34. For example, in one embodiment, the engineered polypeptide comprises the amino acid sequence set forth in SEQ ID NO:22 or a sequence at least 90% identical thereto. In another embodiment, the engineered polypeptide comprises the amino acid sequence set forth in SEQ ID NO:23 or a sequence at least 90% identical thereto. In another embodiment, the engineered polypeptide comprises the amino acid sequence set forth in SEQ ID NO:24 or a sequence at least 90% identical thereto. In another embodiment, the engineered polypeptide comprises the amino acid sequence set forth in SEQ ID NO:25 or a sequence at least 90% identical thereto. In another embodiment, the engineered polypeptide comprises the amino acid sequence set forth in SEQ ID NO:26 or a sequence at least 90% identical thereto. In another embodiment, the engineered polypeptide comprises the amino acid sequence set forth in SEQ ID NO:27 or a sequence at least 90% identical thereto. In another embodiment, the engineered polypeptide comprises the amino acid sequence set forth in SEQ ID NO:28 or a sequence at least 90% identical thereto. In another embodiment, the engineered polypeptide comprises the amino acid sequence set forth in SEQ ID NO:29 or a sequence at least 90% identical thereto. In another embodiment, the engineered polypeptide comprises the amino acid sequence set forth in SEQ ID NO:30 or a sequence at least 90% identical thereto. In another embodiment, the engineered polypeptide comprises the amino acid sequence set forth in SEQ ID NO:31 or a sequence at least 90% identical thereto. In another embodiment, the engineered polypeptide comprises the amino acid sequence set forth in SEQ ID NO:32 or a sequence at least 90% identical thereto. In another embodiment, the engineered polypeptide comprises the amino acid sequence set forth in SEQ ID NO:33 or a sequence at least 90% identical thereto. In another embodiment, the engineered polypeptide comprises the amino acid sequence set forth in SEQ ID NO:34 or a sequence at least 90% identical thereto.

In another embodiment, the engineered polypeptide that specifically binds to human serum albumin consists of an amino acid sequence selected from the group consisting of SEQ ID NOS: 22-34 and fragments thereof. For example, in one embodiment, the engineered polypeptide consists of the amino acid sequence set forth in SEQ ID NO:22. In another embodiment, the engineered polypeptide consists of the amino acid sequence set forth in SEQ ID NO:23. In another embodiment, the engineered polypeptide consists of the amino acid sequence set forth in SEQ ID NO:24. In another embodiment, the engineered polypeptide consists of the amino acid sequence set forth in SEQ ID NO:25. In another embodiment, the engineered polypeptide consists of the amino acid sequence set forth in SEQ ID NO:26. In another embodiment, the engineered polypeptide consists of the amino acid sequence set forth in SEQ ID NO:27. In another embodiment, the engineered polypeptide consists of the amino acid sequence set forth in SEQ ID NO:28. In another embodiment, the engineered polypeptide consists of the amino acid sequence set forth in SEQ ID NO:29. In another embodiment, the engineered polypeptide consists of the amino acid sequence set forth in SEQ ID NO:30. In another embodiment, the engineered polypeptide consists of the amino acid sequence set forth in SEQ ID NO:31. In another embodiment, the engineered polypeptide consists of the amino acid sequence set forth in SEQ ID NO:32. In another embodiment, the engineered polypeptide consists of the amino acid sequence set forth in SEQ ID NO:33. In another embodiment, the engineered polypeptide consists of the amino acid sequence set forth in SEQ ID NO:34.

In another embodiment, the disclosure provides an engineered polypeptide that specifically binds to human serum albumin, wherein the polypeptide comprises three complementarity determining regions, CDR1, CDR2 and CDR3, wherein CDR1 comprises any one of the amino acid sequences of SEQ ID NOs: 35-43 or a sequence that is at least 90% identical to SEQ ID Nos: 35-43; CDR2 comprises any one of the amino acid sequences of SEQ ID NOs: 44-51 or a sequence that is at least 90% identical to SEQ ID Nos: 44-51; and CDR3 comprises any one of the amino acid sequences of SEQ ID NOs: 52-63 or a sequence that is at least 90% identical to SEQ ID Nos: 52-63.

The engineered polypeptide disclosed herein can specifically bind, for example, to the same epitope on human serum albumin as Alb1 (AVOLVESGGG LVQPGNSLRL SCAASGFTFR SFGMSWVROA PGKEPEWVSS ISGSGSDTLY ADSVKGRETI SRDNAKTTLY LOMNSLKPED TAVYYCTIGG SLSRSSOGTQ VTVSS; SEQ ID NO: 149). In other embodiments, the engineered polypeptide competitively inhibits the binding of Alb 1 to human serum albumin.

When the engineered polypeptide comprises an Ig, a suitable fragment of the Ig, such as an Ig variable domain, may also be used in place of a full Ig.

Methods for identifying CDRs from within a given immunoglobulin variable domain are known in the art (Wu, T. & Kabat, E., J. Exp. Med., 132: 211-50, 1970; Clothia, C. et al., Nature, 342: 877-83, 1989; Al-Lazikani, B. et al., J. Mol. Biol., 273: 927-48, 1997; and Ofran, Y. et al., J. Immunol., 181: 6230-35, 2008).

### Fusion Proteins That Specifically Bind Complement Component C5 and Serum Albumin

Described herein are fusion proteins that comprise engineered polypeptides that specifically bind albumin and complement component C5, wherein the engineered polypeptides are fused directly or are linked via one or more suitable linkers or spacers. The term “peptide linker” as used herein refers to one or more amino acid residues inserted or included between the engineered polypeptides of the fusion protein(s). The peptide linker can be, for example, inserted or included at the transition between the engineered polypeptides of the fusion protein at the sequence level. The identity and sequence of amino acid residues in the linker may vary depending on the desired secondary structure. For example, glycine, serine and alanine are useful for linkers having maximum flexibility. Any amino acid residue can be considered as a linker in combination with one or more other amino acid residues, which may be the same as or different from the first amino acid residue, to construct larger peptide linkers as necessary depending on the desired properties. In other embodiments, the linker is GGGGAGGGGAGGGGS (SEQ ID NO:102). In other embodiments, the linker is GGGGSGGGGSGGGGS (SEQ ID NO:103). Additional peptide linkers suitable for use in creating fusion proteins described herein include, for example, G4S (SEQ ID NO:104), (G4S) 2 (SEQ ID NO:105), (G4S) 3 (SEQ ID NO:106), (G₄S)₄ (SEQ ID NO:107), (G₄S)₅ (SEQ ID NO:108), (G₄S)₆ (SEQ ID NO:109), (EAAAK)₃ (SEQ ID NO:110), PAPAP (SEQ ID NO:111), G₄SPAPAP (SEQ ID NO:112), PAPAPGAS (SEQ ID NO:113), GSTSGKSSEGKG (SEQ ID NO:114), (GGGDS)₂ (SEQ ID NO:115), (GGGES)₂ (SEQ ID NO: 116), GGGDSGGGGS (SEQ ID NO:117), GGGASGGGGS (SEQ ID NO:118), GGGESGGGGS (SEQ ID NO:119), ASTKGP (SEQ ID NO:120), ASTKGPSVFPLAP (SEQ ID NO:121), G₃P (SEQ ID NO:122), G-P (SEQ ID NO:123), PAPNLLGGP (SEQ ID NO: 124), G₆ (SEQ ID NO: 125), G₁₂ (SEQ ID NO:126), APELPGGP (SEQ ID NO: 127), SEPQPQPG (SEQ ID NO:128), (G₃S₂)₃ (SEQ ID NO:129), ATTORNEY-DOCKET NO.: 51196-005003 SUBSTITUTE SPECIFICATION GGGGGGGGGGGGS (SEQ ID NO:130), GGGGSGGGGGGGGGS (SEQ ID NO: 131), (GGSSS)₃ (SEQ ID NO:132), (GS₄)₃ (SEQ ID NO:133), G₄A(G₄S)₂ (SEQ ID NO: 134), G₄SG₄AG₄S (SEQ ID NO:135), G₃AS (G₄S)₂ (SEQ ID NO:136), G₄SG₃SG₄S (SEQ ID NO:137), G₄SAG₃SG₄S (SEQ ID NO:138), (G₄S)₂AG₃S (SEQ ID NO:139), G₄SAG₃SAG₃S (SEQ ID NO:140), G₄D(G₄S)₂ (SEQ ID NO:141), G₄SG₄DG₄S (SEQ ID NO: 142), (G₄D)₂G₄S (SEQ ID NO:143), G₄E(G₄S)₂ (SEQ ID NO:144), G₄SG₄EG₄S (SEQ ID NO:145) and (G₄E)₂G₄S (SEQ ID NO:146). One of skill in the art can select a linker, for example, to reduce or eliminate post-translational modification, e.g., glycosylation, e.g., xylosylation. In certain embodiments, the fusion protein comprises at least two sdAbs, Dabs, VHH antibodies, VHH antibody fragments, or combination thereof wherein at least one of the sdAbs, Dabs, VHH antibodies, or VHH antibody fragments is directed against albumin and one of the sdAbs, Dabs, VHH antibodies, or VHH antibody fragments is directed against complement component C5, so that the resulting fusion protein is multivalent or multi-specific. The binding domains or moieties can be directed against, for example, HSA, cynomolgus monkey serum albumin, human C5 and/or cynomolgus monkey C5.

In some embodiments, the C-terminal residue of the albumin-binding domain of the fusion protein can be fused either directly or via a peptide to the N-terminal residue of the complement component C5 binding domain. In other embodiments, the C-terminal residue of the complement component C5 binding domain of the fusion protein can be fused either directly or via a peptide to the N-terminal residue of the albumin-binding domain.

In some embodiments, a fusion protein comprises a complement component C5 binding comprising an amino acid sequences of SEQ ID NOs: 1-12 or a fragment thereof;

and the polypeptide that specifically binds to human serum albumin can comprise an amino acid sequence of SEQ ID NOs: 22-34 or a fragment thereof. In some embodiments, the first polypeptide is derived from an amino acid sequence set forth in any of SEQ ID NOs: 1-12 and the second polypeptide is derived from an amino acid sequence set forth in any of SEQ ID NOs: 22-34. The human complement component C5-binding domain can comprise, for example, the amino acid sequence of SEQ ID NO:5 or 11, and the albumin-binding domain can comprise, for example the amino acid sequence of SEQ ID NO:26. In another embodiment, the disclosure provides a fusion protein having any one of the amino acid sequences of SEQ ID NOs: 64-95. In another embodiment, the disclosure provides a fusion protein having the amino acid sequence of SEQ ID NO:93. In another embodiment, the disclosure provides a fusion protein having the amino acid sequence of SEQ ID NO:77. In another embodiment, the disclosure provides for a fusion protein having any one of the amino acid sequences of SEQ ID NOs: 96-101.

The fusion proteins disclosed herein can be made by expressing in a host cell at least one nucleic acid molecule comprising a nucleotide sequence encoding the fusion protein. Host cells can be mammalian, plant or microbial in origin. In addition to known mammalian host cells, yeast host cells, e.g., Pichia pastoris, Saccharomyces cerevisiae, and/or plant host cells can be used.

### Therapeutic Compositions Comprising Polypeptides That Specifically Bind Complement C5 or Serum Albumin, or Fusion Proteins Thereof, and Administration Thereof

In another embodiment, the disclosure provides engineered polypeptides comprising or consisting of an amino acid sequence as disclosed herein. In another embodiment, the disclosure provides fusion proteins and multivalent and multi-specific fusion proteins comprising or consisting of at least one engineered polypeptide of the disclosure that is linked to at least one therapeutic or targeting moiety, optionally via one or more suitable linkers or spacers.

The disclosure further relates to therapeutic uses of the engineered polypeptides of the disclosure, or fusion proteins and multivalent and multi-specific fusion proteins comprising or consisting of such engineered polypeptides, or to pharmaceutical compositions comprising such engineered polypeptides, fusion proteins, or multivalent and multi-specific fusion proteins.

In some embodiments, the therapeutic or targeting moiety can comprise, for example, at least one sdAb, Dab, VHH or fragment(s) thereof. In certain embodiments, the engineered polypeptide of the disclosure is a multivalent and/or multi-specific fusion protein comprising at least two sdAbs, Dabs, VHH antibodies, VHH antibody fragments, or combination(s) thereof.

In some embodiments, the engineered polypeptides, fusion proteins, or multivalent and multi-specific fusion proteins show an affinity for HSA that is higher than the affinity for mouse serum albumin. In certain embodiments, the engineered polypeptides, fusion proteins, or multivalent and multi-specific fusion proteins show an affinity for cynomolgus monkey serum albumin that is higher than the affinity for mouse serum albumin. In other embodiments, the engineered polypeptides, fusion proteins, or multivalent and multi-specific fusion proteins show an affinity for HSA that is higher than the affinity for cynomolgus monkey serum albumin.

In some embodiments, the engineered polypeptides, fusion proteins, or multivalent and multi-specific fusion proteins show an affinity for human C5 that is higher than the affinity for mouse C5. In certain embodiments, the engineered polypeptides, fusion proteins, or multivalent and multi-specific fusion proteins show an affinity for cynomolgus monkey C5 that is higher than the affinity for mouse C5. In other embodiments, the engineered polypeptides, fusion proteins, or multivalent and multi-specific fusion proteins show an affinity for human C5 that is higher than the affinity for cynomolgus monkey C5.

The engineered polypeptides, fusion proteins, or multivalent and multi-specific fusion proteins described herein can exhibit, for example, improved therapeutic properties, including, for example, increased efficacy, bioavailability, half-life or other therapeutically desirable properties when compared to antibody therapeutics or other therapeutics. In one embodiment, a fusion protein of the disclosure comprises at least one engineered polypeptide disclosed herein and at least one therapeutic or targeting moiety. In such fusion proteins, the fusion protein can exhibit, for example, an increased half-life compared to the therapeutic binding domain alone. Generally, such fusion proteins have a half-life that is at least 1.5 times, or at least 2 times, or at least 5 times, or at least 10 times, or more than 20 times greater than the half-life of the corresponding therapeutic or targeting moiety alone. In some embodiments, a fusion protein of the disclosure has a half-life that is increased by more than 1 hour, more than 2 hours, more than 6 hours, or more than 12 hours as compared to the half-life of the corresponding therapeutic or targeting moiety. In other embodiments, a fusion protein has a half-life that is more than 1 hour, more than 2 hours, more than 6 hours, more than 12 hours, about one day, about two days, about one week, about two weeks, about three weeks, or no more than 2 months.

The term “half-life,” as used herein, refers to the time taken for the serum concentration of the engineered polypeptide, fusion protein, or multivalent and multi-specific fusion protein to be reduced by 50%, in vivo, as a result, for example, of the degradation of the molecule and/or clearance or sequestration of the molecule by physiological mechanisms. Methods for pharmacokinetic analysis and determination of half-life are known to those skilled in the art.

A general description of multivalent and multi-specific fusion proteins containing one or more VHH antibodies and their preparation are known (Els Conrath, K. et al., J. Biol. Chem., 276: 7346-50, 2001; Muyldermans, S., J. Biotechnol., 74: 277-302 2001; International Publication Nos. WO 96/34103, WO 99/23221 and WO 04/041865).

The engineered polypeptides, fusion proteins, and multivalent and multi-specific fusion proteins disclosed herein can be expressed from or associated with constructs that include, for example, one or more elements such as expression vectors (WO 04/041862).

The engineered polypeptides, fusion proteins, and multivalent and multi-specific fusion proteins disclosed herein can be expressed in, for example, isolated host cells comprising nucleic acid molecules that encode the engineered polypeptides, fusion proteins, and multivalent and multi-specific fusion proteins disclosed herein. Suitable host cells include but are not limited to mammalian and yeast cells.

The therapeutic or pharmaceutical compositions disclosed herein can comprise a therapeutically effective amount of one or more engineered polypeptides, fusion proteins, or multivalent and multi-specific fusion proteins as disclosed herein in admixture with a pharmaceutically or physiologically acceptable formulation agent selected for suitability with the mode of administration. Acceptable formulation materials are preferably nontoxic to recipients at the dosages and concentrations to be employed.

Acceptable formulation materials can be used to modify, maintain, or preserve, for example, the pH, osmolarity, viscosity, clarity, color, isotonicity, odor, sterility, stability, rate of dissolution or release, adsorption, or penetration of the composition.

Acceptable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton;

tromethamine; lecithin; cholesterol or tyloxapal), stability enhancing agents (such as sucrose or sorbitol), tonicity enhancing agents (such as alkali metal halides—preferably sodium or potassium chloride-or mannitol sorbitol), delivery vehicles, diluents, excipients and/or pharmaceutical adjuvants (see, e.g., REMINGTON′S PHARMACEUTICAL SCIENCES (18th Ed., A. R. Gennaro, ed., Mack Publishing Company 1990), and subsequent editions of the same, which are incorporated herein by reference).

A skilled artisan can develop a pharmaceutical composition comprising the engineered polypeptides, fusion proteins, or multivalent and multi-specific fusion proteins disclosed herein depending upon, for example, the intended route of administration, delivery format, and desired dosage.

Since the engineered polypeptides, fusion proteins, and multivalent and multi-specific fusion proteins disclosed herein can exhibit, for example, an increased half-life, they may, in some embodiments, be administered to be in circulation. As such, they can be administered in any suitable manner, such as intravenously, subcutaneously, via injection or infusion, or in any other suitable manner that allows the engineered polypeptides, fusion proteins, or multivalent and multi-specific fusion proteins to enter circulation. The preparation of such pharmaceutical compositions is within the knowledge of one of skill in the art.

Any of the engineered polypeptides, fusion proteins, and multivalent and multi-specific fusion proteins disclosed herein, can be administered in combination with an additional therapy, i.e., combined with other agents. The term “coadministered” as used herein includes any or all of simultaneous, separate, or sequential administration of the engineered polypeptides, fusion proteins, and multivalent and multi-specific fusion proteins described herein with adjuvants and other agents, including administration as part of a dosing regimen.

Pharmaceutical compositions described herein can include one or more agents to improve, for example, delivery of the therapeutic agent. Additional agents can be co-administered, for example, as a co-injectable. Agents that degrade hyaluronan, for example, can be included in the pharmaceutical compositions described herein, or such agents can be co-administered with the pharmaceutical compositions described herein to facilitate, for example, dispersion and absorption of the therapeutic agents described herein upon administration. An example of such an agent is recombinant hyaluronidase.

The pharmaceutical compositions can also be selected for parenteral delivery. Alternatively, the compositions can be selected for inhalation or for delivery through the digestive tract, such as orally. The preparation of such pharmaceutical compositions is within the knowledge of one of skill in the art.

Additional pharmaceutical compositions will be evident to those of skill in the art, including formulations involving sustained-delivery or controlled-delivery formulations. Techniques for formulating sustained-delivery or controlled-delivery formulations, using, for example, liposome carriers, bio-erodible microparticles or porous beads, and depot injections, are known to those of skill in the art.

The disclosure also encompasses therapeutic kits comprising the engineered polypeptides, fusion proteins, and multivalent and multi-specific fusion proteins disclosed herein. In some embodiments, the kits comprise both a first container having a dried protein and a second container having an aqueous formulation. In other embodiments, the kits comprise single and multi-chambered pre-filled syringes (e.g., liquid syringes and lyosyringes).

The disclosure also encompasses an article of manufacture comprising a container comprising a label and a composition comprising the engineered polypeptides, fusion proteins, and multivalent and multi-specific fusion proteins disclosed herein wherein the label indicates that the composition is to be administered to a patient having, or that is suspected of having, a complement-mediated disorder.

In one embodiment, the disclosure provides a method for preventing and/or treating at least one disease, condition, or disorder that can be prevented or treated using an engineered polypeptide, fusion protein, or multivalent and multi-specific fusion protein disclosed herein, the method comprising administering to a patient in need thereof a therapeutically or pharmaceutically effective amount of an engineered polypeptide, fusion protein, or multivalent and multi-specific fusion protein disclosed herein. In particular embodiments, the disorder is a complement-mediated disorder such as, for example, rheumatoid arthritis (RA); lupus nephritis; asthma; ischemia-reperfusion injury; atypical hemolytic uremic syndrome (aHUS); dense deposit disease (DDD); paroxysmal nocturnal hemoglobinuria (PNH); macular degeneration (e.g., age-related macular degeneration (AMD); hemolysis, elevated liver enzymes, and low platelets (HELLP) syndrome; Guillain-Barré Syndrome (GBS); CHAPLE syndrome; myasthenia gravis (MG); neuromyelitis optica (NMO); post-hematopoietic stem cell transplant thrombotic microangiopathy (post-HSCT-TMA); post-bone marrow transplant TMA (post-BMT TMA); Degos disease; Gaucher's disease; glomerulonephritis; thrombotic thrombocytopenia purpura (TTP); spontaneous fetal loss; Pauci-immune vasculitis; epidermolysis bullosa; recurrent fetal loss; multiple sclerosis (MS); traumatic brain injury; and injury resulting from myocardial infarction, cardiopulmonary bypass and hemodialysis.

The effective amount of a pharmaceutical composition as disclosed herein to be employed therapeutically will depend, for example, upon the therapeutic context and objectives. One of skill in the art will appreciate that an appropriate dosage level for treatment will vary depending, in part, upon the molecule being delivered, the indication for which the composition is being used, the route of administration, and the size (body weight, body surface, or organ size) and condition (age and general health) of the patient.

### EXAMPLES

The Examples that follow are illustrative of specific embodiments of the disclosure, and various uses thereof. They are set forth for explanatory purposes only, and should not be construed as limiting the scope of the invention in any way.

### Example 1. Llama Immunization and Anti-C5 VHH Phage Library Construction

Llama immunizations were performed starting with a primary injection followed by secondary boosts. Briefly, primary immunization was initiated with 500 μg of human complement protein C5 and subsequent 500 μg human complement protein C5 antigen boosts administered at week 2 (boost 1), week 4 (boost 2), week 8 (boost 3), and week 12 (boost 4). Serum titers were measured by ELISA and titers after boost 3 were found to be the highest-10-fold above the pre-bleed signal at the 1:1,000,000 dilution. Peripheral blood mononuclear cells (PBMCs) were isolated from blood samples after boost 3. Cell viability was found to be 98% by trypan blue staining. Cells were lysed in RNA lysis buffer immediately after PBMC isolation. Total RNA was isolated from PBMCs and cDNA was synthesized using llama heavy chain specific primers. VHH (heavy chain only) fragments were separated from VH (conventional heavy chain) fragments via gel electrophoresis. The VHH fragments were cloned into pADL-10b (Antibody Design Labs, San Diego, CA), and the DNA library was transformed into TG1 cells. 114 colonies were randomly sequenced and 101 (89%) correct sequences were obtained. The library was scraped and suspended in 25% glycerol, then stored at −80C.

### Example 2. Phage Display Panning and Screening for Anti-C5 VHH Domains

TG1 cells containing the anti-human complement protein C5 VHH domain library were grown to logarithmic phase (OD₆₀₀=0.4-0.8) at 37C in 2×YT media containing 100 μg/mL carbenicillin and 2% glucose. The cells were infected with M13K07 helper phage with and without shaking at 37C for 30 minutes. Infected cells were pelleted at 4000× g for 10 minutes and resuspended in 2×YT media containing 100 μg/mL carbenicillin, 50 μg/mL kanamycin, and 1 mM IPTG, and the bacteriophage was propagated by overnight growth at 30C and 250 rpm. The overnight culture was centrifuged at 9000× g for 10 minutes at 4C, and phage was precipitated with one-fifth volume of a PEG-NaCl solution [20% polyethyleneglycol 6000, 1.5 M NaCl] by incubation for 1 hour on ice. Phage particles were pelleted by centrifugation at 9000× g for 15 minutes at 4C and the supernatant was discarded. Phage particles were resuspended in superblock blocking buffer and cell debris was pelleted by centrifugation for 10 minutes at 7500× g in a microcentrifuge tube. The supernatant containing phage particles was transferred to a new tube and phage was precipitated again as described above. Concentrated phage particles were subjected to a thermal challenge for 1 hour at 70C, and the phage titer before and after heating was determined by infection of logarithmic phase TG1 cells followed by plating on 2×YT agar plates with 100 g/mL carbenicillin, 50 g/mL kanamycin, and 2% glucose.

The library selection strategy included selection with biotinylated cynomolgus monkey (cyno) complement protein C5 and competition with molar equivalent non-biotinylated human complement protein C5 to obtain affinity matched anti-C5 VHH domains with reactivity to both human and cyno species. The phage display VHH library was subjected to a deselection step against Dynabeads® M-280 streptavidin for 1 hour at room temperature. The deselected phage particles were selected for matched affinity to human and cyno C5 by incubating in an equimolar solution of biotinylated cyno C5 and non-biotinylated human C5 with Dynabeads® M-280 Streptavidin for 30 minutes at room temperature. After 5 rounds of washing with PBST and PBS, phage was eluted off the beads using 0.1 M glycine (pH 2.2) with 1 mg/mL BSA. The eluted supernatant was neutralized with 1 M Tris pH 8.0. Log phase TG1 cells were infected with the neutralized phage and plated on 2YTCG medium to measure the output titer. Output and input titers were compared to calculate the enrichment ratio; a higher ratio suggested the successful isolation of C5 specific clones.

Individual clones were picked, inoculated in a 96-well deep well plate in 2×YT media with 100 μg/mL carbenicillin and 2% glucose, and grown to log phase. The cells were infected with M13K07 and cultured overnight at 30C for the production of phage particles displaying individual VHH domains in culture supernatant. Phage ELISA screening of four 96-well plates with human C5 captured on streptavidin-coated plates suggested ˜60% positive clones. 72 unique clones out of a total of 76 were selected as representatives based on sequence analysis of CDR H3. The sequences of these representative VHH clones are provided in Table 1. For cloning purposes, the N-and C-terminal amino acids were modified to match the N-and C-terminal amino acids of human VH-3 germline.

Amino acid sequences suitable for use in the engineered polypeptides of the disclosure include the amino acid sequences disclosed in Tables 1 or fragments thereof.

TABLE 1
Representative llama-derived anti-C5 VHH domains and
whether each clone binds to human complement protein
C5 (hC5) and/or cyno complement protein C5 (cC5).

| VHH | | Binds | Binds |
|---|---|---|---|
| domain | Sequence | hC5 | cC5 |
| LCP0081 | EVQLVESGGGLVQTGGSLRLSCAASTSGSDFSGKKMAWYRQAPGNGRE | + | - |
| | FVAIIFSNKVTDYADSVKGRFTISRDNAKKTVYLQMSSLTPTDTAVYY | | |
| | CHDQEISWGQGTQVTVSS (SEQ ID NO: 150) | | |
| LCP0082 | EVQLVESGGGLVQAGGSLRLSCAASGTSVVINSMGWYRQAPGKQRELV | + | + |
| | ATIDLSGTTNYADSAQGRFTISRDNAENLNLVYLQMNNLNPDDTAVYY | | |
| | CNALLSRAVSGSYVYWGQGTQVTVSS (SEQ ID NO: 151) | | |
| LCP0083 | EVQLVESGGGLVQPGGSLRLSCTSRIGTISNIDLMNWYRQAPGKQREF | + | + |
| | VASLQSNGATNYADSVKGRFTISRDNAKNTLFLQMNSLNPEDTAVYFC | | |
| | HALLPRSPYNSWGQGTQVTVSS (SEQ ID NO: 152) | | |
| LCP0085 | EVQLVESGGGLVQAGGSLRLSCAASSIIPNIYAMGWYRQAPGKQRELV | + | + |
| | ASIENGLPANYADSVKGRFTISRDNAKNTVFLQMHSLKSEDTAVYYCY | | |
| | AFRPGVPTTWGQGTQVTVSS (SEQ ID NO: 153) | | |
| LCP0086 | EVQLVESGGGLVQAGESLRLSCAASGSISAINAMGWYRQAPGKQREFV | + | − |
| | ADITRAGVSDYADAVKGRFTISRDNAKNTFYLQMNDLKPEDTAVYYCD | | |
| | ALLIAGGVYWGQGTQVTVSS (SEQ ID NO: 154) | | |
| LCP0088 | EVQLVESGGGLVQAGGSLRLSCTASGRTISTTVMGWFRQAPGKEREFV | + | + |
| | AAVHWGDGNTVYADSVKGRFTISRDDAKNTVYLQLNYLKPEDTSVYYC | | |
| | AARPPTYVGTSRNSRSYDYWGQGTQVTVSS (SEQ ID NO: 155) | | |
| LCP0089 | EVQLVESGGGLVQAGGSLRLSCVVSGRAIDRNAMGWFRQAPGKERESV | + | − |
| | AAISASSGNTYYSDSVTGRFTISRDNTKNTVYLQMNSLKPEDTAVYYC | | |
| | AAGSRGSWYLFDRREYDYWGQGTQVTVSS (SEQ ID NO: 156) | | |
| LCP0090 | EVQLVESGGGLVQAGGSLRLTCTASETSFDINVMGWYRQAPGKQRELV | + | + |
| | AIITASGNTEYADSAKGRFTISRDNTKNTVAMQMNNLKPDDTAVYYCY | | |
| | VLLSGAVSGVYAHWGQGTQVTVSS (SEQ ID NO: 157) | | |
| LCP0091 | EVQLVESGGGLVQAGGSLTLSCAASGRTDSRYAMGWFRQAPGKERELM | + | + |
| | AAISWSGRPTYYADSVKGRFTISRDNAKNTVSLQMNSLKPEDTAVYYC | | |
| | AYKRLPAWYTGSAYYSQESEYDYWGQGTQVTVSS (SEQ ID | | |
| | NO: 158) | | |
| LCP0092 | EVQLVESGGGLVQPGGSLRLSCTSRIGTISNIDLMNWYRQAPGKQREF | + | + |
| | VASLQSTGTTDYADSVKGRFTISRDNAKNTLFLQMNSLNPEDTAVYYC | | |
| | HALIPRSPYNVWGQGTQVTVSS (SEQ ID NO: 159) | | |
| LCP0095 | EVQLVESGGGLVQAGGSLRLSCTASGRTISTTVMAWFRQAPGKEREFV | + | + |
| | AADHWGDAGTVYADSVKGRFTISRDNAKNTVYLQMNYLKPEDTSVYYC | | |
| | AARPPTYVGTSRDSRAYDYWGQGTQVTVSS (SEQ ID NO: 160) | | |
| LCP0097 | EVQLVESGGGLVQPGGSLRLSCAASESISSDSPMAWYRQAPGKQREMV | + | + |
| | ARILPIGPPDYADAVKDRFSISRENAKNTVYLQMNSLKPEDTAVYYCN | | |
| | LLHLPSGLNYWGQGTQVTVSS (SEQ ID NO: 161) | | |
| LCP0098 | EVQLVESGGDLVQAGGSLRLSCVASRSISSAMNWYRQPPGKQRELVAL | + | − |
| | ITRGFNTNYADSVKGRFTISRDNAKNTVYLQMNSLKPEDTGVYYCNSL | | |
| | NYWGQGTQVTVSS (SEQ ID NO: 162) | | |
| LCP0100 | EVQLVESGGGLVQAGGSLRLSCAASGRTDSMWSMGWFRQAPGQEREFV | + | − |
| | AAISWSVGTYYEDSVKGRFTLSRDDDKDTAYLEMSDLKLEDTADYYCA | | |
| | ASTRHGTNLVLPRDYDYWGQGTQVTVSS (SEQ ID NO: 163) | | |
| LCP0101 | EVQLVESGGGLVQPGGSLRLSCTSRIGTISNIDLMNWYRQAPGKQREF | + | + |
| | VASLQSTGTTDYADSVKGRFTISRDNAKNTLFLQMNSLNPEDTAVYYC | | |
| | HALLPRSPYNAWGQGTQVTVSS (SEQ ID NO: 164) | | |
| LCP0102 | EVQLVESGGGLVQAGGSLRLSCAASGIIPNIYAMGWYRQAPGKQRELV | + | + |
| | ASIENGGSTNYADSVKGRFTISRDNARNTVFLQMHSLKSEDTAVYYCY | | |
| | AFRPGVPTDWGQGTQVTVSS (SEQ ID NO: 165) | | |
| LCP0103 | EVQLVESGGGLVQAGGSLTLSCVASGRTFSNYRMGWFRQAPGAEREFV | + | + |
| | GTIYWSTGRSYYGDSVKGRFIISGDNAKNTIHLQMNSLKPEDTGVYYC | | |
| | ASGPENSAFDSWGQGTQVTVSS (SEQ ID NO: 166) | | |
| LCP0104 | EVQLVESGGGLVQAGDSLRLSCAASGRPFSSYTMGWFRQAPGKERDFV | + | − |
| | ATISWSGGIKYYADSVEGRFSISRDNAKNMVYLQMNSLKPEDTAVYYC | | |
| | AATELRTWSRQTFEYDYWGQGTQVTVSS (SEQ ID NO: 167) | | |
| LCP0105 | EVQLVESGGGLVQAGGSLRLSCTASGRTISTTVMAWFRQAPGKEREFV | + | + |
| | AAVHWGDESTVYADSVKGRFTISRDNAKNTVYLQMNYLKPEDTSVYYC | | |
| | AARPPTYVGSSRSSRAYDYWGQGTQVTVSS (SEQ ID NO: 168) | | |
| LCP0106 | EVQLVESGGGLVQAGGSLRLSCVVSGSILDINVMAWYRQAPGKQREFV | + | + |
| | ARITSGGDIDYADPVKGRFTISTNGAKNTVYLQMNSLKPEDTAAYYCN | | |
| | VLLSRSSAGRYTHWGQGTQVTVSS (SEQ ID NO: 169) | | |
| LCP0111 | EVQLVESGGGLVQPGGSLRLSCAASGFPFSLYDMGWYRQAPEKQRESV | + | − |
| | AIITQSGSTDYADSVKGRFTISRDNAKNTLYLQMNSLKPEDTAVYYCR | | |
| | LVGVTWGQGTQVTVSS (SEQ ID NO: 170) | | |
| LCP0112 | EVQLVESGGGLVQAGGSLTLSCAASGRTFSSYGIGWFRQAPGKEREFV | + | − |
| | AAISRTGQTTHYADSIRFTISRDNAKNTVYLQMNSLKPEDTAVYYCAA | | |
| | RTGGPIYGSEYHYWGQGTQVTVSS (SEQ ID NO: 171) | | |
| LCP0113 | EVQLVESGGGLVQAGDSLTLSCAASGRPFSSLTMGWFRQAPGKGREFV | + | − |
| | ATTSWSGDIKYYADFVKGRFTISRDNAKNMVYLQMNSLKPEDTAVYYC | | |
| | AATLLRTWSRQTNEYEYWGQGTQVTVSS (SEQ ID NO: 172) | | |
| LCP0114 | EVQLVESGGGLVQPGGSLRLSCTSRIGTISNIDLMNWYRQAPGKQREF | + | + |
| | VASLQSTGTTDYADSVRGRFTISRDNAKNTLFLQMNSLNPEDTAVYYC | | |
| | HALLPRSPYNVWGQGTQVTVSS (SEQ ID NO: 173) | | |
| LCP0115 | EVQLVESGGGLVQAGGSLRLSCAASGRTFSGILSPYAVGWFRQAPGKG | + | + |
| | REFVSTITSGGSAIYTDSVKGRFTLSRDNAKDTVYLQMNSLKPEDTAV | | |
| | YYCAVRTRRYGSNLGEVPQENEYGYWGQGTQVTVSS (SEQ ID | | |
| | NO: 174) | | |
| LCP0122 | EVQLVESGGGLVQAGGSLRLSCAAPETGATINVMAWYRQAPGKQRELV | + | + |
| | ARVAIDNNTDYADHAKGRFTISRDNTKNTVYLQMNNLKPDDTAVYYCN | | |
| | VLLSRQISGSYGHWGQGTQVTVSS (SEQ ID NO: 175) | | |
| LCP0123 | EVQLVESGGGLVQAGGSLTLSCAMSGGTRPFEDYVMAWFRQATGKERE | + | + |
| | FVATITWMGETTYYKDSVNGRFAISRDNAENTVALQMNSLEPEDTAVY | | |
| | FCAAHSRSSFSTSGGRYNPRPTEYDYWGQGTQVTVSS (SEQ ID | | |
| | NO: 176) | | |
| LCP0125 | EVQLVESGGGLVQAGGSLRLSCTASGRTISTTVMGWFRQAPGKEREFV | + | + |
| | AAVHWGDEGTVYADSVKGRFTISRDNAKNTVYLQMNALKPEDTSVYYC | | |
| | AAKPPTYVGTSRSSRAYVYWGQGTQVTVSS (SEQ ID NO: 177) | | |
| LCP0126 | EVQLVESGGGLVQAGDSLTLSCAASGSGFSINVMAWYRQAPGKQRDLV | + | + |
| | ASMTIGGRTNYKDSLKGRFTISRDNTKNTAYLQMNSLKPEDTAVYYCY | | |
| | ALLDRGIGGNYVYWGQGTQVTVSS (SEQ ID NO: 178) | | |
| LCP0127 | EVQLVESGGGLVQAGGSLRLSCAASGLTFSDYYMGWFRQAPGKERDFL | + | + |
| | ARIGKSGIGKSYADSVRGRFTISRDNAKNTVYLQMNNLKLEDTAVYYC | | |
| | AADRDIAYDARLTAEYDYWGQGTQVTVSS (SEQ ID NO: 179) | | |
| LCP0128 | EVQLVESGGGLVQAGGSLRLSCTASGRTISTTVMGWFRQAPGKEREFV | + | − |
| | AAVHWGDESTVYADSVKGRFTISRDNAKNTVYLQMNYLKPEDTAVYYC | | |
| | AARPPTYVGTSRSSRAYDYWGQGTQVTVSS (SEQ ID NO: 180) | | |
| LCP0129 | EVQLVESGGGLVQAGGSLRLSCAASVASETIVSINDMAWYRQAPGKQR | + | + |
| | ELVASITIHNNRDYADSAKGRFTISRDDTKNTVYLQMTHLKPDDTAVY | | |
| | YCTVLLSRALSGSYRFWGQGTQVTVSS (SEQ ID NO: 181) | | |
| LCP0130 | EVQLVESGGGLVQAGGSLRLSCTGSETSGTIFNINVMGWYRQAPGKQR | ND | ND |
| | ELVAIMDIGGTTDYADSVKGRFTISRDNAKNTVYVQMNNLKSEDTAVY | | |
| | YCYCALDRAVAGRYTYWGQGTQVTVSS (SEQ ID NO: 182) | | |
| LCP0132 | EVQLVESGGGLVQPGGSLRLSCEASGISLNDYNMGWFRQAPGKDREIV | + | - |
| | AALSRRSHGIYQSDSVKYRFSISRDNTKNMVSLQMDSLRPEDTAVYYC | | |
| | AADGDPYFTGRDMNPEYWGQGTQVTVSS (SEQ ID NO: 183) | | |
| LCP0133 | EVQLVESGGGSVQAGGSLRLSCAFSGGRFSDYGMAWFRQGPGKEREFV | + | + |
| | SRISGNGRGTQYTDSVSGRFIISRDNDKNTVYLQMNDLKVEDTAIYYC | | |
| | ARGSGPSSFNEGSVYDYWGQGTQVTVSS (SEQ ID NO: 184) | | |
| LCP0134 | EVQLVESGGGLVQSGGSLTLSCVLSGSIFSSNTMGWHRQAPGKQREWV | + | + |
| | AITTSGGTTKYADSVKGRFTISRDNAKNTVYLRMNNLKPEDTGVYFCY | | |
| | ASLAGIWGQGTQVTVSS (SEQ ID NO: 185) | | |
| LCP0135 | EVQLVESGGGLVQAGGSLRLSCAAPETEATYNVMGWYRRAPGKQRELV | + | + |
| | ATMTIDYNTNYADSAKGRFTISRDNTKNTVYLQMNNLRPDDTAVYYCR | | |
| | VDLSRQISGSYNYWGQGTQVTVSS (SEQ ID NO: 186) | | |
| LCP0136 | EVQLVESGGGLVQPGESLRLSCAISGFAFTDVGMSWVRQAPGKGLEWV | + | + |
| | SSISSGSSITTYSDSVKGRFTISRDNARNTLFLQMNSLKPEDTAVYYC | | |
| | GRYYCTGLGCHPRRDSALWGQGTQVTVSS (SEQ ID NO: 187) | | |
| LCP0137 | EVQLVESGGGLVQPGGSLRLSCRASGFTYSTAAMGWVRQAPGKGLEWV | + | + |
| | SSISSLGSDRKSADSVKGRFTISRDNAKNTLYLQMNSLKPEDTAVYYC | | |
| | ARFISNRWSRDVHAPSDFGSRGQGTQVTVSS (SEQ ID NO: 188) | | |
| LCP0138 | EVQLVESGGGSVPAGGSLRLSCAAFGFTFDNYAIAWFRQAPGKEREGV | + | − |
| | SCLSTNDGETYYADSVKGRFTISSDHAKNTVYLQMDSLRPEDTAVYYC | | |
| | AAAEGSWCHKYEYDYWGQGTQVTVSS (SEQ ID NO: 189) | | |
| LCP0139 | EVQLVESGGGLVQAGESLRLSCAASGRTSDLYVVGWFRQTPGKEREFV | + | − |
| | AGIAWTGDASYYADSVEGRFTIARDNAENRIDLQMTSLKPEDTAVYYC | | |
| | AADSRARFERQRYNDMNYWGQGTQVTVSS (SEQ ID NO: 190) | | |
| LCP0141 | EVQLVESGGGLVQAGGSLRLSCIASVTIADINVMGWYRQAPGKQREFV | + | + |
| | ASIPTTGDKNYAESAKGRFTISRDNSQNTVAMQMNNLKPDDTAVYYCY | | |
| | VLLSRAVSGSYGHWGQGTQVTVSS (SEQ ID NO: 191) | | |
| LCP0142 | EVQLVESGGGLVQVGGSLRLSCAASGSIVDIKVMGWYRQAPGNERELV | + | + |
| | ALINDADDSEYSPSMRGRFTISRDNSKNTVYLQMNSLKPEDTAAYYCA | | |
| | ADRDSSWFKSPYIPGSWGQGTQVTVSS (SEQ ID NO: 192) | | |
| LCP0143 | EVQLVESGGGLVQAGGSLRLSCAAPEMGATINVMAWYRQAPGKQRELV | + | + |
| | ARLPLDNNIDYGDFAKGRFTISRDITRNTVYLQMNNLKPDDTAVYYCN | | |
| | VLLSRQINGAYVHWGQGTQVTVSS (SEQ ID NO: 193) | | |
| LCP0144 | EVQLVESGGGLVQAGGSLRLSCAASGIDGDINVMAWYRQAPGKQRELV | + | + |
| | ASITIGGNTNYADSVKGRFTIARDNAKNRMSLEMNSLKSEDTAVYYCN | | |
| | TLLSRVHDGQYVFWGQGTQVTVSS (SEQ ID NO: 194) | | |
| LCP0145 | EVQLVESGGGLVQAGGSLRLSCVASEDAFKTDTLGWFRQAPGEEREFV | + | − |
| | AAFVWAGGPFYADSVKGRFTISMDEDRNTVYLQMNSLKPEDTGVYYCA | | |
| | ASLSRLRVGEITPRHMNYWGQGTQVTVSS (SEQ ID NO: 195) | | |
| LCP0146 | EVQLVESGGGLVQAGGSLRLSCAASGRAFSDYAMAWFRQAPGKEREFV | + | + |
| | AGIGWSGGDTLYADSVRGRFTNSKDNAKNRMSLQMNSLKPEDTAVYYC | | |
| | AARQGQYIYSSMRSDSYDYWGQGTQVTVSS (SEQ ID NO: 196) | | |
| LCP0147 | EVQLVESGGGLVQAGGSLRLSCAASGRTFSSSNMGWFRQAPGEEREFV | + | + |
| | TAIDWSGGRTYYADSVKGRFTISRDNAKNTVYLQMDSLKPEDTAVYYC | | |
| | AAQGSGLDWGYPWTYDYWGQGTQVTVSS (SEQ ID NO: 197) | | |
| LCP0149 | EVQLVESGGGLVQPGGSLKLSCATSGSVLNIDSMAWYRQAPGKQRELV | + | − |
| | AEMLWGGTKNYGDSVKGRFTISGDADWGTELQMSSLKPEDTAVYYCNA | | |
| | VGRGFRDAWGQGTQVTVSS (SEQ ID NO: 198) | | |
| LCP0150 | EVQLVESGGGLVQAGGSLRLSCVASGSGFGILDMGWYRQAPGSRRELV | + | + |
| | GYVTRDGTTNYGNSVKGRSIISEDITKNTVILQMNSLKPEDTAVYFCT | | |
| | AGLTNQPRAWGQGTQVTVSS (SEQ ID NO: 199) | | |
| LCP0151 | EVQLVESGGGLVQPGGSLRLSCAASGSVSSINVMGWYRQTPGKQRELV | + | + |
| | AAINRGGSTNVADSVKGRFTISRDNAKNTVYLQMNSLKPEDTAVYYCN | | |
| | AEPYGLDWRYDYWGQGTQVTVSS (SEQ ID NO: 200) | | |
| LCP0152 | EVQLVESGGGLEQAGGSLRLSCTASGGTDSIYQMGWFRQTPGKEREFV | + | − |
| | AAINWNYGGAYYPDSVKGRFTISRDKAKNIGFLQMNSLKPEDTAVYYC | | |
| | ATSQTSVDAFSVPITTARRYQYWGQGTQVTVSS (SEQ ID | | |
| | NO: 201) | | |
| LCP0153 | EVQLVESGGGLVQAGGSLTLSCVASGRTFSNYRMGWFRQAPGKEREFV | + | + |
| | GTIYWSTGRSYYGDSVKGRFIISGDNAKNTIHLQMNSLKPGDTGVYYC | | |
| | ASGPEMSAFDSWGQGTQVTVSS (SEQ ID NO: 202) | | |
| LCP0154 | EVQLVESGGGLVQPGGSLRLSCAASGFTLDDYAIGWFRQAPGKEREGV | + | + |
| | SCISSSDGSTYYGDSVKGRFTISRDNAKNTMYLQMNSLKPEDTAVYYC | | |
| | ATGTPLSSYYGSCLDYDMAYWGQGTQVTVSS (SEQ ID NO: 203) | | |
| LCP0155 | EVQLVESGGGLVQAGGSLRLSCAASGVTFSNYGMAWFRQAPEKEREFV | + | + |
| | ARISSNGRRTEYADGVSGRFTISRDNAKNTVYLQMNGLKPEDTAVYYC | | |
| | ARAAGPSGFHEQSIYDDWGQGTQVTVSS (SEQ ID NO: 204) | | |
| LCP0295 | EVQLVESGGGLVQAGGSLRLSCAVSGRSISTYVAGWFRQGPGKEREFV | + | + |
| | ALISRGGGDIQYSDSVKGRFTISRDNAKNAVYLQMNSLKPADTAVYYC | | |
| | SLDASFGSRLVSRWDYWGQGTQVTVSS (SEQ ID NO: 205) | | |
| LCP0296 | EVQLVESGGGVVQAGDSLTLTCTAPVGTISDYGMGWFRQAPGKEREFV | + | + |
| | ASISWGGMWTDYADSVKGRFTISRDNDKNAVYLRMNSLNAEDTAVYYC | | |
| | GRGRMYRGIGNSLAQPKSYGYWGQGTQVTVSS (SEQ ID NO: 206) | | |
| LCP0297 | EVQLVESGGGLVQAGGSLRLSCAGSGFTSDDYAIAWFRQAPGKEREGV | + | + |
| | SCIGSGDGTTYYADSVKGRFIISSENAKKTVYLQMNSLKPEDTGIYYC | | |
| | AADLYPPADYALDHTWYDYWGQGTQVTVSS (SEQ ID NO: 207) | | |
| LCP0298 | EVQLVESGGGVVQPGGSLRLSCVVSGSRFSLDTVGWHHQAPGKLRELV | + | + |
| | ARIRDDGDTMYVASVKGRFIISRDDAKNTVYLQMNSLKPEDTGVYYCY | | |
| | FSRNGAWGQGTQVTVSS (SEQ ID NO: 208) | | |
| LCP0299 | EVQLVESGGGLVQAGGSLRLSCGASGRISDINVMGWYRQAPGKQREMV | + | + |
| | ADIDIRGYTNYADSVKGRFTVSRDNAETMYLEMNSLKPEDTAVYRCNA | | |
| | LTSRDWGTGKYVYWGQGTQVTVSS (SEQ ID NO: 209) | | |
| LCP0300 | EVQLVESGGDLVQVGGSLRLSCAFPGSMSSRNSVNWYRQPPGKQREWV | + | + |
| | ATISVSGFTQYADSAKGRFTISRDSAKNTVHLQMNSLKPEDTGVYYCN | | |
| | YMDYWGQGTQVTVSS (SEQ ID NO: 210) | | |
| LCP0301 | EVQLVESGGGVVRAGGSLKLSCTAAGTDINIVTVGWHRQAPGKHRELV | + | + |
| | ATIVGSGSRTNYADSVKGRFTISRDNPKNTVYLQMNSLKPEDTAVYYC | | |
| | YATSIGWGQGTQVTVSS (SEQ ID NO: 211) | | |
| LCP0302 | EVQLVESGGGLVQAGGSLRLSCAASGRTFSGILSAYAVGWFRQAPGKE | + | + |
| | REFVSTITSGGSTLSADSVKGRFTLSRDNAKDTVYLQMNSLKPEDTAV | | |
| | YYCAVRTWPYGSNRGEVPTENEYGHWGQGTQVTVSS (SEQ ID | | |
| | NO: 212) | | |
| LCP0303 | EVQLVESGGGSVQAGGSLRLTCTASGNVRSIFTMAWYRQAPGKQRELV | + | + |
| | ASAAKGGDTYYADSAKGRFTISRDDAKAIVSLQMNSLKPEDTAVYYCK | | |
| | TDGRPWFSEDYWGQGTQVTVSS (SEQ ID NO: 213) | | |
| LCP0304 | EVQLVESGGGLVQVGDSMRLSCAVFGNIFTRDPVMWFRQPPGKQREWV | + | + |
| | ATITPSGFANYADSVKGRFTISRYAANNTVHLQMNSLKPEDTGVYFCN | | |
| | FGTYWGQGTQVTVSS (SEQ ID NO: 214) | | |
| LCP0306 | EVQLVESGGGLVQAGGSLRLSCAASKGAFNINVMAWYRQAPGKQRELV | + | + |
| | ARVALGGTTDYADSVKGRFTISRNNAQDTVYLQMNSLKPEDTAVYYCN | | |
| | VLLDRGVRGSYAYWGQGTQVTVSS (SEQ ID NO: 215) | | |
| LCP0309 | EVQLVESGGGLVQAGGSLRLSCAASGRTYSSYVIGWFRQAPGKEREFV | + | + |
| | ASIRWAGGDSHYQESVKGRSTISKDNARNTVYLQMNSLKPEDTAVYYC | | |
| | AGAAPVPGQSYEWSSWGQGTQVTVSS (SEQ ID NO: 216) | | |
| LCP0310 | EVQLVESGGGLVQAGGSLRLSCVASGSAFYVGPMAWYRQAPGKERESV | + | + |
| | ASITKGGITNYADSVKGRFTISRDNAKNTVYLQMNSLKPEDTDVYVCN | | |
| | ARVKLQEDRLFRDYWGQGTQVTVSS (SEQ ID NO: 217) | | |
| LCP0311 | EVQLVESGGGMVQPGGSLRLSCVVSGASGNIDFVTVGWHRQAPGKHRE | + | + |
| | MVAVITGDGTRNYRDSVKGRFSISRDNAKNTIYLQMNSLKPEDTAVYY | | |
| | CYMSNPISSWGQGTQVTVSS (SEQ ID NO: 218) | | |
| LCP0312 | EVQLVESGGGLVQAGGSRRLSCAVSGRTLSSFGMGWFRQAPEKPREFV | + | + |
| | AAITWGQGGTFYADSVKGRFTISRDIVKNTVYLQMNDLKPDDTGLYFC | | |
| | VSAPHFHEAFPSRPPAYAYWGQGTQVTVSS (SEQ ID NO: 219) | | |
| LCP0313 | EVQLVESGGGLVQAGGSLRLSCAASGRTYGSYVIGWFRQAPGKEREFV | + | + |
| | ASIRWAGGDSHYGDPLKGRSTISKDNAKNTVYLQMNSLKPEDAAVYYC | | |
| | AGAAPVPGSSYEWTNWGQGTQVTVSS (SEQ ID NO: 220) | | |
| LCP0314 | EVQLVESGGGLVQAGGSLRLSCAASGSISSVNTMGWYRQAPGKQRELV | + | + |
| | AFITSGDDTNYADSMKGRFTISRDNAKNTLYLQMNSLKPEDTAVYYCV | | |
| | ATLGRSSSGTYTYWGQGTQVTVSS (SEQ ID NO: 221) | | |
| LCP0316 | EVQLVESGGGLVQAGGSLRLSCAASLRTLDNYGVGWFRQTPGREREFV | + | + |
| | SAVSWNGDRTYYQDSVKGRFTISREYAKNTVYLQMDSLKPEDTAVYYC | | |
| | AVNMYGSTFPGLSVESHYDYWGQGTQVTVSS (SEQ ID NO: 222) | | |
| LCP0317 | EVQLVESGGGLVQAGGSLRLSCAASGSIFSINAMAWYRQAQGKQRELV | + | + |
| | ADITKNDITDYADSVKGRFTIARDNAKNTVDLQMNSLKPEDTAVYYCT | | |
| | AALSRHPYRSWGQGTQVTVSS (SEQ ID NO: 223) | | |
| LCP0319 | EVQLVESGGGLVQAGGSLRLSCAAAGRSLSDYYIIWFRQPPGKEYEFV | + | + |
| | SSIRWNTGSTTYGDSVKGRFTISRDNAKSTVYLQMNSLKPEDTALYWC | | |
| | AAGLHLTPTSRTYNYRGQGTQVTVSS (SEQ ID NO: 224) | | |
| LCP0320 | EVQLVESGGGLVQAGGSLRLSCAAPETIFTINSMGWYRQAPGKQRELV | + | + |
| | AFINLDGNTNYADSAKGRFTISRDNAENTVYLQMDNLKPDDTAVYYCN | | |
| | VLLSRAISGSYVHWGQGTQVTVSS (SEQ ID NO: 225) | | |

### Example 3. Cloning and Expression of Anti-C5 VHH Domains

Representative anti-C5 VHH domains were subcloned into a mammalian expression vector and expressed as VHH-His-tag fusions in Expi293F cells. Culture supernatants were harvested when cell viability dropped to 50-60%. The supernatants were analyzed via SDS-PAGE under reducing conditions, followed by Coomassie brilliant blue staining. Expression levels were calculated using biolayer interferometry on an Octet (ForteBio Inc.) instrument. His-tagged VHH domains were purified by Immobilized Metal Affinity Chromatography (IMAC) on an AKTA (GE Healthcare) from the culture supernatants.

### Example 4. Binding and Functional Analysis of Anti-C5 VHH Domains

Binding analysis to complement component C5. Representative anti-C5 VHH domains were sequenced, characterized, and evaluated for binding to human, cynomolgus monkey (cyno), and mouse C5 protein using Biolayer Interferometry on an Octet (ForteBio Inc.) instrument. Cell culture supernatants from expressed VHH-His domains were normalized to a concentration of 20 μg/mL in 2× kinetics buffer and loaded on anti-penta-HIS (HIS1K) biosensor tips (ForteBio Inc.) for 300 seconds to fully saturate the sensor tips. The saturated tips were then exposed to a solution containing 50 nM of soluble C5 (human, cyno or mouse) in 2× kinetics buffer each for 600 seconds in separate experiments and dissociation was followed for 600 seconds into 2× kinetics buffer. VHH domains that showed binding to human (hC5) or cyno C5 (cC5) are marked with a ‘+’ in Table 1.
Hemolysis assays for C5 antagonism. A hemolysis assay measures the release of hemoglobin from sensitized chicken erythrocytes lysed on exposure to Complement Classical Pathway (CCP)-activated serum. His-tagged VHH domains were expressed in Expi293 cells. Preliminary assays were used to select functional anti-C5 VHH domains, which were purified by IMAC. Ten purified VHH domains were analyzed for their ability to inhibit CCP-mediated hemolysis of sensitized chicken erythrocytes at different concentrations.

No antibody and 20 mM EDTA were used as complete lysis and no lysis controls for the assay, respectively. The ten VHH domains and the control anti-C5 IgGs (denoted h5G1.1, BNJ441 and Ec-CHO) at different concentrations (32 μg/mL to 0.5 μg/mL) were pre-incubated with 20% normal human serum (NHS) in 0.1 mL gelatin veronal buffered saline (GVB++, cat #B100, Comptech) for 30 minutes at room temperature. 400 μL chicken erythrocytes (Lampire Biologicals, cat #7201403) were washed four times with 1 mL of GVB++and sensitized cRBCs were prepared by incubating 5×10⁷ cells/mL with 1:500 (v/v) dilution of rabbit-anti-chicken IgG (cat #203-4139, Rockland) and incubated at 4C for 15 minutes. The cells were washed twice with GVB++ and resuspended in a final volume of 3.6 mL GVB++. 30 μL of sensitized cRBCs (2.5×10⁶ cells) were added to the pre-incubated human serum and antibodies, and incubated at 37C for 30 minutes. The cells were pelleted by centrifugation at 1700× g for 3 minutes at 4C and the supernatant (85 μL) was transferred to a new flat bottom 96 well plate. Absorbance was measured at 415 nm. Percent lysis was calculated for each VHH domain and the control antibodies as:

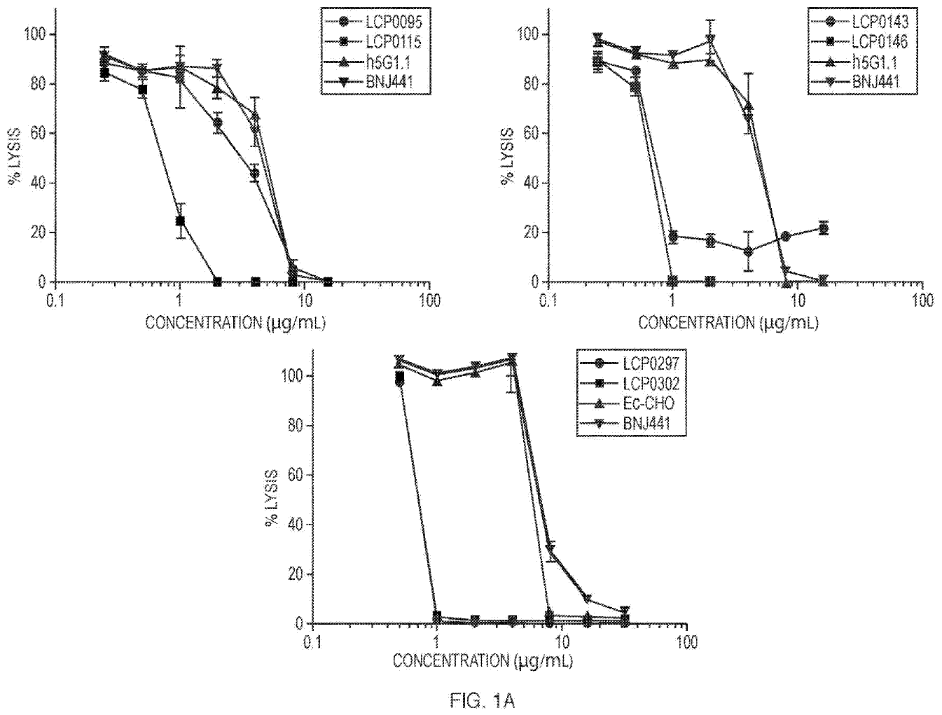

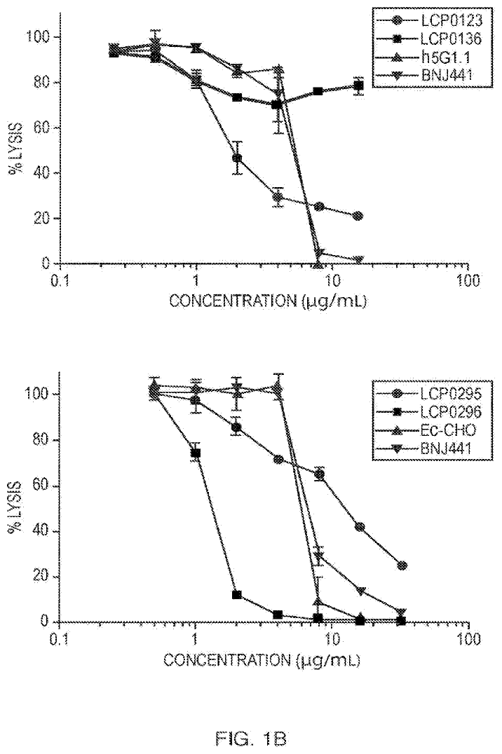

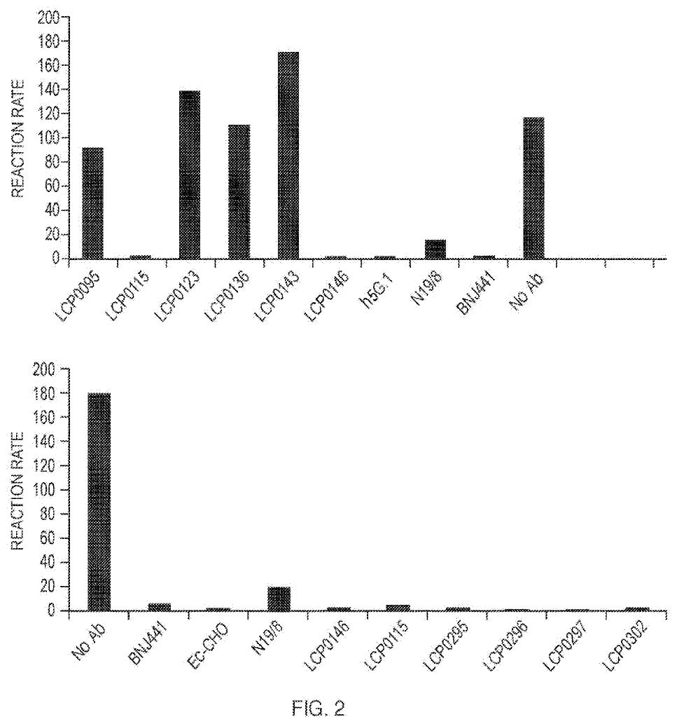

((A₄₁₅ₛₐₘₚₗₑ−A_{415 no lysis})/(A_{415complete lysis}−A_{415 no lysis}))×100

where A₄₁₅ₛₐₘₚₗₑ is the absorbance at 415 nm for the sample antibody, A_{415no lysis} is the absorbance at 415 nm for no lysis control (20 mM EDTA), and A_{415 complete lysis} is the absorbance at 415 nm for complete lysis control. The results are shown in FIG. 1.
Identification of VHH domains that inhibit C5a liberation. Human C5 protein cleavage (e.g., C5a liberation with Complement Alternative Pathway C5 convertase deposited on CAP-activator Zymosan) was measured using a Meso Scale Discovery (MSD)-based immunoassay. Anti-C5 VHH domains were expressed and purified as in the previous section and were analyzed for their ability to block the cleavage of human C5 protein by measuring the amount of hC5a released. Optimal concentration for the sample VHH domain was determined in pilot experiments. The sample VHH domains and control antibodies (h5G1.1, N19/8, BNJ441 and Ec-CHO) were added to human C5 protein (final concentration 25 nM) (CompTech Inc.) in GVB++ buffer containing 1% gelatin, and 2.5 mM NiCl for 30 minutes at 37C and stored at 4C until further use. A MSD high-binding 96 well plate was coated with an anti-C5a antibody at 2 μg/mL in BupH Phosphate Buffered Saline (ThermoFisher) and incubated for 1 hour. Zymosan was then added to NHS in equal proportion to activate the complement alternative pathway. This mixture of zymosan-NHS was then added to pre-incubated VHH-hC5 solution and incubated at 37C. The reaction was stopped at different time points (0, 30, 60 and 90 minutes) by addition of futhan-EDTA. The plate was centrifuged at 3600 rpm for 2 minutes and supernatant was transferred to a new polypropylene plate. Blocker A was added for 1 hour at room temperature to block non-specific binding to the coated MSD plate. The MSD plate was washed and supernatant from samples from above were added. This plate was incubated at room temperature for 15 minutes. A mixture of detection antibody biotin-Ab2942 (Abcam) at 1 μg/mL and streptavidin conjugated sulfo tag at 0.5 μg/mL was prepared and then added to each well and incubated at room temperature for 30 minutes. MSD 2× read buffer was added to each well and the electro-chemiluminscent signal was measured. Raw data was analyzed using the MSD workbench software. The results from this experiment are shown in FIG. 2.

LCP0115, LCP0146, LCP0295, LCP0296, LCP0297 and LCP0302 inhibited the release of C5a and were used for further characterization.

### Example 5. Affinity Analysis of Anti-C5 VHH Domains by Biacore

Anti-C5 VHH domains were prioritized based on cross reactivity to cyno C5 and eight purified anti-C5 VHH domains were subjected to affinity analysis by Biacore. The kinetic parameters for binding to human and cyno C5 for the initial eight candidates are shown in Table 2. Out of the eight affinity-analyzed candidates, five anti-C5 domains (LCP0115, LCP0143, LCP0146, LCP0296, and LCP0302) were chosen and prioritized for humanization and further analysis based on matched affinity to human and cyno C5.

TABLE 2
Results of Biacore characterization of VHH domains.

| Sample | C5 | kₐ (1/Ms) | k_{d} (1/s) | K_{D} (M) | Chi² |
|---|---|---|---|---|---|
| LCP0095 | hC5 | 2.86e5 | 7.14e−4 | 2.50e−9 | 6.94 |
| | cC5 | 4.56e5 | 1.68e−3 | 3.69e−9 | 12.9 |
| LCP0115 | hC5 | 1.13e5 | 3.48e−5 | 3.09e−10 | 0.08 |
| | cC5 | 9.53e4 | 1.02e−5 | 1.07e−10 | 0.10 |
| LCP0123 | hC5 | 1.08e5 | 2.16e−4 | 1.99e−9 | 0.13 |
| | cC5 | 1e5 | 3.81e−4 | 3.8e−9 | 0.14 |
| LCP0136 | hC5 | 4.86e5 | 8.82e−4 | 1.81e−9 | 2.47 |
| | cC5 | 7.89e5 | 2.51e−4 | 3.18e−10 | 1.01 |
| LCP0143 | hC5 | 6.91e5 | 5.66e−5 | 8.2e−11 | 0.90 |
| | cC5 | 7.41e5 | 1.24e−4 | 1.67e−10 | 0.81 |
| LCP0146 | hC5 | 2.24e6 | 9.75e−5 | 4.35e−11 | 0.42 |
| | cC5 | 2.64e6 | 2.44e−4 | 9.22e−11 | 0.47 |
| LCP0296 | hC5 | 9.34e4 | 3.9e−5 | 4.17e−10 | 0.06 |
| | cC5 | 6.84e4 | 1.06e−4 | 1.55e−9 | 0.03 |
| LCP0302 | hC5 | 1.14e5 | 2.22e−5 | 1.95e−10 | 0.03 |
| | cC5 | 1.03e5 | 2.38e−5 | 2.32e−10 | 0.03 |

### Example 6. Humanization of Anti-C5 VHH Domains

Five prioritized anti-C5 VHH domains (LCP0115, LCP0143, LCP0146, LCP0296 and LCP0302) were humanized by CDR grafting onto human germlines with sequence similarity to the llama sequence. CDRs were based on higher amino acid coverage among the IMGT and Kabat definitions. Back mutations to llama FR2 hallmark residues were made to maintain VHH domain stability. The humanized variants were expressed in Expi293 cells and tested for binding to human C5 using biolayer interferometry.

Further back mutations to parental llama residues were introduced in selected frameworks for several of the variants to improve their affinity for human C5. Constructs were expressed in HEK293F cells and evaluated for binding by biolayer interferometry. Additional mutations were made in some of the variants to further optimize their affinity, and the N-termini were humanized to EVQLV (SEQ ID NO:147; where necessary) and the C-termini were humanized to WGQGTLVTVSS (SEQ ID NO:148; where necessary). Resulting prioritized anti-C5 VHH candidates are shown in Table 3 below. The CDRs from these candidates are shown in Table 4.

TABLE 3
Humanized anti-C5 VHH domain candidates

| VHH anti-C5 | | |
|---|---|---|
| candidate | | |
| name | Candidate sequence | SEQ ID NO: |
| LCP0177 | EVQLVESGGGLVQPGGSLRLSCAASGRTFSGILSPYAVGWFRQ | 226 |
| | APGQGLEAVATITSGGSAIYTDSVKGRFTISRDNSKNTLYLQM | |
| | NSLRAEDTAVYYCAVRTRRYGSNLGEVPQENEYGYWGQGTLVT | |
| | VSS | |
| LCP0178 | EVQLVESGGGLVQPGGSLRLSCAASEMGATINVMAWFRQAPGQ | 227 |
| | GLEAVARLPLDNNIDYGDFAKGRFTISRDNSKNTLYLQMNSLR | |
| | AEDTAVYYCNVLLSRQINGAYVHWGQGTLVTVSS | |
| LCP0179 | EVQLVESGGGLVQPGGSLRLSCAASGRAFSDYAMAWFRQAPGQ | 228 |
| | GLEAVAGIGWSGGDTLYADSVRGRFTISRDNSKNTLYLQMNSL | |
| | RAEDTAVYYCAARQGQYIYSSMRSDSYDYWGQGTLVTVSS | |
| LCP0180 | EVQLVESGGGLVQPGGSLRLSCAASGRTFSGILSPYAVGWFRQ | 229 |
| | APGQGREFVATITSGGSAIYTDSVKGRFTISRDNSKNTLYLQM | |
| | NSLRAEDTAVYYCAVRTRRYGSNLGEVPQENEYGYWGQGTLVT | |
| | VSS | |
| LCP0181 | EVQLVESGGGLVQPGGSLRLSCAAPEMGATINVMAWYRQAPGQ | 230 |
| | QRELVARLPLDNNIDYGDFAKGRFTISRDNSKNTLYLQMNSLR | |
| | AEDTAVYYCNVLLSRQINGAYVHWGQGTLVTVSS | |
| LCP0182 | EVQLVESGGGLVQPGGSLRLSCAASGRAFSDYAMAWFRQAPGQ | 231 |
| | EREFVAGIGWSGGDTLYADSVRGRFTISRDNSKNTLYLQMNSL | |
| | RAEDTAVYYCAARQGQYIYSSMRSDSYDYWGQGTLVTVSS | |
| LCP0183 | EVQLVESGGGLVQPGGSLRLSCAASGRTFSGILSPYAVGWFRQ | 232 |
| | APGKGREFVSTITSGGSAIYTDSVKGRFTISRDNAKNSLYLQM | |
| | NSLRAEDTAVYYCAVRTRRYGSNLGEVPQENEYGYWGQGTLVT | |
| | VSS | |
| LCP0184 | EVQLVESGGGLVQPGGSLRLSCAASGRTFSGILSPYAVGWFRQ | 233 |
| | APGKGLEFVSTITSGGSAIYTDSVKGRFTISRDNAKNSLYLQM | |
| | NSLRAEDTAVYYCAVRTRRYGSNLGEVPQENEYGYWGQGTLVT | |
| | VSS | |
| LCP0185 | EVQLVESGGGLVKPGGSLRLSCAASEMGATINVMAWYRQAPGK | 234 |
| | QRELVSRLPLDNNIDYGDFAKGRFTISRDNAKNSLYLQMNSLR | |
| | AEDTAVYYCNVLLSRQINGAYVHWGQGTLVTVSS | |
| LCP0186 | EVQLVESGGGLVKPGGSLRLSCAASEMGATINVMAWYRQAPGK | 235 |
| | GLELVSRLPLDNNIDYGDFAKGRFTISRDNAKNSLYLQMNSLR | |
| | AEDTAVYYCNVLLSRQINGAYVHWGQGTLVTVSS | |
| LCP0187 | EVQLVESGGGLVQPGRSLRLSCAASGRAFSDYAMAWFRQAPGK | 236 |
| | EREFVSGIGWSGGDTLYADSVRGRFTISRDNAKNSLYLQMNSL | |
| | RAEDTALYYCAARQGQYIYSSMRSDSYDYWGQGTLVTVSS | |
| LCP0188 | EVQLVESGGGLVQPGRSLRLSCAASGRAFSDYAMAWFRQAPGK | 237 |
| | GLEFVSGIGWSGGDTLYADSVRGRFTISRDNAKNSLYLQMNSL | |
| | RAEDTALYYCAARQGQYIYSSMRSDSYDYWGQGTLVTVSS | |
| LCP0195 | EVQLVESGGGLVQPGGSLRLSCAASGRAFSDYAMAWFRQAPGQ | 1 |
| | EREFVAGIGWSGGDTLYADSVRGRFTNSRDNSKNTLYLQMNSL | |
| | RAEDTAVYYCAARQGQYIYSSMRSDSYDYWGQGTLVTVSS | |
| LCP0197 | EVQLVESGGGLVQPGGSLRLSCAASGRAFSDYAMAWFRQAPGQ | 2 |
| | EREFVAGIGWSGGDTLYADSVRGRFTISRDNAKNTLYLQMNSL | |
| | RAEDTAVYYCAARQGQYIYSSMRSDSYDYWGQGTLVTVSS | |
| LCP0199 | EVQLVESGGGLVQPGGSLRLSCAASGRAFSDYAMAWFRQAPGQ | 3 |
| | EREFVAGIGWSGGDTLYADSVRGRFTISRDNSKNTMYLQMNSL | |
| | RAEDTAVYYCAARQGQYIYSSMRSDSYDYWGQGTLVTVSS | |
| LCP0203 | EVQLVESGGGLVQPGGSLRLSCAASGRAFSDYAMAWFRQAPGQ | 4 |
| | GLEFVAGIGWSGGDTLYADSVRGRFTISRDNSKNTLYLQMNSL | |
| | RAEDTAVYYCAARQGQYIYSSMRSDSYDYWGQGTLVTVSS | |
| LCP0207 | EVQLVESGGGLVQPGGSLRLSCAASGRTFSGILSPYAVGWFRQ | 5 |
| | APGKGLEFVSTITSGGSAIYTDSVKGRFTISRDNAKDSLYLQM | |
| | NSLRAEDTAVYYCAVRTRRYGSNLGEVPQENEYGYWGQGTLVT | |
| | VSS | |
| LCP0208 | EVQLVESGGGLVQPGGSLRLSCAASGRTFSGILSPYAVGWFRQ | 6 |
| | APGKGLEFVSTITSGGSAIYTDSVKGRFTISRDNAKNTLYLQM | |
| | NSLRAEDTAVYYCAVRTRRYGSNLGEVPQENEYGYWGQGTLVT | |
| | VSS | |
| LCP0209 | EVQLVESGGGLVQPGGSLRLSCAASGRTFSGILSPYAVGWFRQ | 7 |
| | APGKGLEFVSTITSGGSAIYTDSVKGRFTISRDNAKNSVYLQM | |
| | NSLRAEDTAVYYCAVRTRRYGSNLGEVPQENEYGYWGQGTLVT | |
| | VSS | |
| LCP0212 | EVQLVESGGGLVQPGGSLRLSCAASGRTFSGILSPYAVGWFRQ | 8 |
| | APGQGLEFVATITSGGSAIYTDSVKGRFTISRDNSKNTLYLQM | |
| | NSLRAEDTAVYYCAVRTRRYGSNLGEVPQENEYGYWGQGTLVT | |
| | VSS | |
| CRL0303 | EVQLVESGGGLVQPGGSLRLSCAASGRHFSDYAMAWFRQAPGQ | 9 |
| | EREFVAGIGWSGGDTLYADSVRGRFTISRDNSKNTLYLQMNSL | |
| | RAEDTAVYYCAARQGQYIYSSMRSDSYDYWGQGTLVTVSS | |
| CRL0304 | EVQLVESGGGLVQPGGSLRLSCAASGRAHSDYAMAWFRQAPGQ | 10 |
| | EREFVAGIGWSGGDTLYADSVRGRFTISRDNSKNTLYLQMNSL | |
| | RAEDTAVYYCAARQGQYIYSSMRSDSYDYWGQGTLVTVSS | |
| CRL0305 | EVQLVESGGGLVQPGGSLRLSCAASGRAHSDYAMAWFRQAPGQ | 11 |
| | EREFVAGIGWSGGDTLYADSVRGRFTNSRDNSKNTLYLQMNSL | |
| | RAEDTAVYYCAARQGQYIYSSMRSDSYDYWGQGTLVTVSS | |
| CRL0307 | EVQLVESGGGLVQPGGSLRLSCAASGRHHSDYAMAWFRQAPGQ | 12 |
| | EREFVAGIGWSGGDTLYADSVRGRFTISRDNSKNTLYLQMNSL | |
| | RAEDTAVYYCAARQGQYIYSSMRSDSYDYWGQGTLVTVSS | |
| CRL0726 | EVQLVESGGGLVQPGGSLRLSCAASVGTISDYGMGWFRQAPGQ | 238 |
| | GLEAVASISWGGMWTDYADSVKGRFTISRDNSKNTLYLQMNSL | |
| | RAEDTAVYYCGRGRMYRGIGNSLAQPKSYGYWGQGTLVTVSS | |
| CRL0727 | EVQLVESGGGLVQPGGSLRLSCAASGRTFSGILSAYAVGWFRQ | 239 |
| | APGQGLEAVATITSGGSTLSADSVKGRFTISRDNSKNTLYLQM | |
| | NSLRAEDTAVYYCAVRTWPYGSNRGEVPTENEYGHWGQGTLVT | |
| | VSS | |
| CRL0728 | EVQLVESGGGLVQPGGSLRLSCAASVGTISDYGMGWFRQAPGQ | 240 |
| | EREFVASISWGGMWTDYADSVKGRFTISRDNSKNTLYLQMNSL | |
| | RAEDTAVYYCGRGRMYRGIGNSLAQPKSYGYWGQGTLVTVSS | |
| CRL0729 | EVQLVESGGGLVQPGGSLRLSCAASGRTFSGILSAYAVGWFRQ | 241 |
| | APGQEREFVATITSGGSTLSADSVKGRFTISRDNSKNTLYLQM | |
| | NSLRAEDTAVYYCAVRTWPYGSNRGEVPTENEYGHWGQGTLVT | |
| | VSS | |
| CRL0730 | EVQLVESGGGLVKPGGSLRLSCAASVGTISDYGMGWFRQAPGK | 242 |
| | EREFVSSISWGGMWTDYADSVKGRFTISRDNAKNSLYLQMNSL | |
| | RAEDTAVYYCGRGRMYRGIGNSLAQPKSYGYWGQGTLVTVSS | |
| CRL0731 | EVQLVESGGGLVKPGGSLRLSCAASVGTISDYGMGWFRQAPGK | 243 |
| | GLEFVSSISWGGMWTDYADSVKGRFTISRDNAKNSLYLQMNSL | |
| | RAEDTAVYYCGRGRMYRGIGNSLAQPKSYGYWGQGTLVTVSS | |
| CRL0732 | EVQLLESGGGLVQPGGSLRLSCAASGRTFSGILSAYAVGWFRQ | 244 |
| | APGKEREFVSTITSGGSTLSADSVKGRFTISRDNSKNTLYLQM | |
| | NSLRAEDTAVYYCAVRTWPYGSNRGEVPTENEYGHWGQGTLVT | |
| | VSS | |
| CRL0733 | EVQLLESGGGLVQPGGSLRLSCAASGRTFSGILSAYAVGWFRQ | 245 |
| | APGKGLEFVSTITSGGSTLSADSVKGRFTISRDNSKNTLYLQM | |
| | NSLRAEDTAVYYCAVRTWPYGSNRGEVPTENEYGHWGQGTLVT | |
| | VSS | |
| CRL0960 | QVQLVQSGAEVKKPGASVKVSCKASGRAFSDYAMAWVRQAPGQ | 246 |
| | GLEWMGGIGWSGGDTLYADSVRGYTENFKDRVTMTRDTSTSTV | |
| | YMELSSLRSEDTAVYYCARQGQYIYSSMRSDSYDYWGQGTLVT | |
| | VSS | |
| CRL0961 | QVQLVQSGAEVKKPGASVKVSCKASGRAFSDYAMAWFRQAPGQ | 247 |
| | EREFMGGIGWSGGDTLYADSVRGYTENFKDRVTMTRDTSTSTV | |
| | YMELSSLRSEDTAVYYCARQGQYIYSSMRSDSYDYWGQGTLVT | |
| | VSS | |
| CRL0962 | QVQLVQSGAEVKKPGASVKVSCKASGRAFSDYAMAWFRQAPGQ | 248 |
| | GLEFMGGIGWSGGDTLYADSVRGYTENFKDRVTMTRDTSTSTV | |
| | YMELSSLRSEDTAVYYCARQGQYIYSSMRSDSYDYWGQGTLVT | |
| | VSS | |
| CRL0963 | QVQLVQSGAEVKKPGASVKVSCKASVGTISDYGMGWVRQAPGQ | 249 |
| | GLEWMGSISWGGMWTDYADSVKGYTENFKDRVTMTRDTSTSTV | |
| | YMELSSLRSEDTAVYYCARGRGRMYRGIGNSLAQPKSYGYWGQ | |
| | GTLVTVSS | |
| CRL0964 | QVQLVQSGAEVKKPGASVKVSCKASVGTISDYGMGWFRQAPGQ | 250 |
| | EREFMGSISWGGMWTDYADSVKGYTENFKDRVTMTRDTSTSTV | |
| | YMELSSLRSEDTAVYYCARGRGRMYRGIGNSLAQPKSYGYWGQ | |
| | GTLVTVSS | |
| CRL0965 | QVQLVQSGAEVKKPGASVKVSCKASVGTISDYGMGWFRQAPGQ | 251 |
| | GLEFMGSISWGGMWTDYADSVKGYTENFKDRVTMTRDTSTSTV | |
| | YMELSSLRSEDTAVYYCARGRGRMYRGIGNSLAQPKSYGYWGQ | |
| | GTLVTVSS | |
| CRL0966 | QVQLVQSGAEVKKPGASVKVSCKASGRTFSGILSAYAVGWVRQ | 252 |
| | APGQGLEWMGTITSGGSTLSADSVKGYTENFKDRVTMTRDTST | |
| | STVYMELSSLRSEDTAVYYCARAVRTWPYGSNRGEVPTENEYG | |
| | HWGQGTLVTVSS | |
| CRL0967 | QVQLVQSGAEVKKPGASVKVSCKASGRTFSGILSAYAVGWFRQ | 253 |
| | APGQEREFMGTITSGGSTLSADSVKGYTENFKDRVTMTRDTST | |
| | STVYMELSSLRSEDTAVYYCARAVRTWPYGSNRGEVPTENEYG | |
| | HWGQGTLVTVSS | |
| CRL0968 | QVQLVQSGAEVKKPGASVKVSCKASGRTFSGILSAYAVGWFRQ | 254 |
| | APGQGLEFMGTITSGGSTLSADSVKGYTENFKDRVTMTRDTST | |
| | STVYMELSSLRSEDTAVYYCARAVRTWPYGSNRGEVPTENEYG | |
| | HWGQGTLVTVSS | |
| CRL0972 | EVQLVESGGGVVRPGGSLRLSFAASGRAFSDYAMAWFRQAPGK | 255 |
| | EREFVSGIGWSGGDTLYADSVRGRFTISRDNAKNSLYLQMNSL | |
| | RAEDTALYHCAARQGQYIYSSMRSDSYDYWGQGTLVTVSS | |
| CRL0973 | EVQLLESGGGLVQPGGSLRLSCAASGRAFSDYAMAWFRQAPGK | 256 |
| | EREFVSGIGWSGGDTLYADSVRGRFTISRDNSKNTLYLQMNSL | |
| | RAEDTAVYYCAARQGQYIYSSMRSDSYDYWGQGTLVTVSS | |
| CRL0974 | EVQLVESGGVVVQPGGSLRLSCAASGRAFSDYAMAWFRQAPGK | 257 |
| | EREFVSGIGWSGGDTLYADSVRGRFTISRDNSKNSLYLQMNSL | |
| | RAEDTALYYCAARQGQYIYSSMRSDSYDYWGQGTLVTVSS | |
| CRL0975 | EVQLVESGGGLVQPGGSLRLSCAASVGTISDYGMGWFRQAPGK | 258 |
| | EREFVSSISWGGMWTDYADSVKGRFTISRDNSKNTLYLQMNSL | |
| | RAEDTAVYYCGRGRMYRGIGNSLAQPKSYGYWGQGTQVTVSS | |
| CRL0976 | EVQLVESGGGLVQPGGSLRLSCAASVGTISDYGMGWFHQAPGK | 259 |
| | EREFVSSISWGGMWTDYADSVKGRFIISRDNSRNTLYLQTNSL | |
| | RAEDTAVYYCGRGRMYRGIGNSLAQPKSYGYWGQGTLVTVSS | |
| CRL0977 | EVQLVESGGGVVQPGRSLRLSCAASVGTISDYGMGWFRQAPGK | 260 |
| | EREFVASISWGGMWTDYADSVKGRFTISRDNSKNTLYLQMNSL | |
| | RAEDTAVYYCGRGRMYRGIGNSLAQPKSYGYWGQGTQVTVSS | |
| CRL0978 | EVQLVESGGGLVKPGGSLRLSCAASGRTFSGILSAYAVGWFRQ | 261 |
| | APGKEREFVSTITSGGSTLSADSVKGRFTISRDNAKNSLYLQM | |
| | NSLRAEDTAVYYCAVRTWPYGSNRGEVPTENEYGHWGQGTQVT | |
| | VSS | |
| CRL0979 | EVQLVESGGGLVQPGGSLRLSCAASGRTFSGILSAYAVGWFRQ | 262 |
| | APGKEREFVSTITSGGSTLSADSVKGRFTISRDNSKNTLYVQM | |
| | SSLRAEDTAVYYCAVRTWPYGSNRGEVPTENEYGHWGQGTQVT | |
| | VSS | |
| CRL0980 | EVQLVESGGGVVQPGGSLRLSCAASGRTFSGILSAYAVGWFRQ | 263 |
| | APGKEREFVSTITSGGSTLSADSVKGRFTISRDNSKNSLYLQM | |
| | NSLRTEDTALYYCAVRTWPYGSNRGEVPTENEYGHWGQGTQVT | |
| | VSS | |

TABLE 4
CDRs of humanized anti-C5 VHH domain candidates

| | CDR1 sequence | CDR2 sequence | CDR3 sequence |
|---|---|---|---|
| VHH domain | [SEQ ID NO:] | [SEQ ID NO:] | [SEQ ID NO:] |
| LCP0146 | GRAFSDYAMA | GIGWSGGDTLYADSVRG | AARQGQYIYSSMRSDSYDY |
| LCP0179 | [13] | [18] | [20] |
| LCP0182 | | | |
| LCP0187 | | | |
| LCP0188 | | | |
| LCP0195 | | | |
| LCP0197 | | | |
| LCP0199 | | | |
| LCP0203 | | | |
| CRL0960 | | | |
| CRL0961 | | | |
| CRL0962 | | | |
| CRL0972 | | | |
| CRL0973 | | | |
| CRL0974 | | | |
| LCP0115 | GRTFSGILSPYAVG | TITSGGSAIYTDSVKG | AVRTRRYGSNLGEVPQENEYGY |
| LCP0177 | [14] | [19] | [21] |
| LCP0180 | | | |
| LCP0183 | | | |
| LCP0184 | | | |
| LCP0207 | | | |
| LCP0208 | | | |
| LCP0209 | | | |
| LCP0212 | | | |
| LCP0143 | EMGATINVMA | RLPLDNNIDYGDFAKG | NVLLSRQINGAYVH |
| LCP0178 | [327] | [325] | [326] |
| LCP0181 | | | |
| LCP0185 | | | |
| LCP0186 | | | |
| CRL0303 | GRHFSDYAMA | GIGWSGGDTLYADSVRG | AARQGQYIYSSMRSDSYDY |
| | [15] | [18] | [20] |
| CRL0304 | GRAHSDYAMA | GIGWSGGDTLYADSVRG | AARQGQYIYSSMRSDSYDY |
| CRL0305 | [16] | [18] | [20] |
| CRL0307 | GRHHSDYAMA | GIGWSGGDTLYADSVRG | AARQGQYIYSSMRSDSYDY |
| | [17] | [18] | [20] |
| LCP0296 | VGTISDYGMG | SISWGGMWTDYADSVKG | GRGRMYRGIGNSLAQPKSYGY |
| CRL0726 | [264] | [266] | [268] |
| CRL0728 | | | |
| CRL0730 | | | |
| CRL0731 | | | |
| CRL0963 | | | |
| CRL0964 | | | |
| CRL0965 | | | |
| CRL0975 | | | |
| CRL0976 | | | |
| CRL0977 | | | |
| LCP0302 | GRTFSGILSAYAVG | TITSGGSTLSADSVKG | AVRTWPYGSNRGEVPTENEYGH |
| CRL0727 | [265] | [267] | [269] |
| CRL0729 | | | |
| CRL0732 | | | |
| CRL0733 | | | |
| CRL0966 | | | |
| CRL0967 | | | |
| CRL0968 | | | |
| CRL0978 | | | |
| CRL0979 | | | |
| CRL0980 | | | |

Back mutations to parental llama residues were introduced in selected frameworks from humanization assessments to improve the affinity of the selected variants. The sequences of the back mutated variants are shown in Table 5. Constructs were expressed in HEK293F cells and evaluated for binding by biolayer interferometry.

TABLE 5
Anti-C5 VHH humanized variants with back mutations

| Variant | | |
|---|---|---|
| name | Back mutated variant sequence | SEQ ID NO |
| LCP0115 | | |
| variants | | |
| LCP0204 | EVQLVESGGGLVQAGGSLRLSCAASGRTFSGILSPYAVGWFRQAPGKGLEF | 270 |
| | VSTITSGGSAIYTDSVKGRFTISRDNAKNSLYLQMNSLRAEDTAVYYCAVR | |
| | TRRYGSNLGEVPQENEYGYWGQGTLVTVSS | |
| LCP0205 | EVQLVESGGGLVQPGGSLRLSCAASGRTFSGILSPYAVGWFRQAPGKGREF | 232 |
| | VSTITSGGSAIYTDSVKGRFTISRDNAKNSLYLQMNSLRAEDTAVYYCAVR | |
| | TRRYGSNLGEVPQENEYGYWGQGTLVTVSS | |
| LCP0206 | EVQLVESGGGLVQPGGSLRLSCAASGRTFSGILSPYAVGWFRQAPGKGLEF | 271 |
| | VSTITSGGSAIYTDSVKGRFTLSRDNAKNSLYLQMNSLRAEDTAVYYCAVR | |
| | TRRYGSNLGEVPQENEYGYWGQGTLVTVSS | |
| LCP0207 | EVQLVESGGGLVQPGGSLRLSCAASGRTFSGILSPYAVGWFRQAPGKGLEF | 5 |
| | VSTITSGGSAIYTDSVKGRFTISRDNAKDSLYLQMNSLRAEDTAVYYCAVR | |
| | TRRYGSNLGEVPQENEYGYWGQGTLVTVSS | |
| LCP0208 | EVQLVESGGGLVQPGGSLRLSCAASGRTFSGILSPYAVGWFRQAPGKGLEF | 6 |
| | VSTITSGGSAIYTDSVKGRFTISRDNAKNTLYLQMNSLRAEDTAVYYCAVR | |
| | TRRYGSNLGEVPQENEYGYWGQGTLVTVSS | |
| LCP0209 | EVQLVESGGGLVQPGGSLRLSCAASGRTFSGILSPYAVGWFRQAPGKGLEF | 7 |
| | VSTITSGGSAIYTDSVKGRFTISRDNAKNSVYLQMNSLRAEDTAVYYCAVR | |
| | TRRYGSNLGEVPQENEYGYWGQGTLVTVSS | |
| LCP0210 | EVQLVESGGGLVQPGGSLRLSCAASGRTFSGILSPYAVGWFRQAPGKGLEF | 272 |
| | VSTITSGGSAIYTDSVKGRFTISRDNAKNSLYLQMNSLKAEDTAVYYCAVR | |
| | TRRYGSNLGEVPQENEYGYWGQGTLVTVSS | |
| LCP0211 | EVQLVESGGGLVQPGGSLRLSCAASGRTFSGILSPYAVGWFRQAPGKGLEF | 273 |
| | VSTITSGGSAIYTDSVKGRFTISRDNAKNSLYLQMNSLRPEDTAVYYCAVR | |
| | TRRYGSNLGEVPQENEYGYWGQGTLVTVSS | |
| LCP0212 | EVQLVESGGGLVQPGGSLRLSCAASGRTFSGILSPYAVGWFRQAPGQGLEF | 8 |
| | VATITSGGSAIYTDSVKGRFTISRDNSKNTLYLQMNSLRAEDTAVYYCAVR | |
| | TRRYGSNLGEVPQENEYGYWGQGTLVTVSS | |
| LCP0146 | | |
| variants | | |
| LCP0193 | EVQLVESGGGLVQAGGSLRLSCAASGRAFSDYAMAWFRQAPGQEREFVAGI | 274 |
| | GWSGGDTLYADSVRGRFTISRDNSKNTLYLQMNSLRAEDTAVYYCAARQGQ | |
| | YIYSSMRSDSYDYWGQGTLVTVSS | |
| LCP0194 | EVQLVESGGGLVQPGGSLRLSCAASGRAFSDYAMAWFRQAPGKEREFVAGI | 275 |
| | GWSGGDTLYADSVRGRFTISRDNSKNTLYLQMNSLRAEDTAVYYCAARQGQ | |
| | YIYSSMRSDSYDYWGQGTLVTVSS | |
| LCP0195 | EVQLVESGGGLVQPGGSLRLSCAASGRAFSDYAMAWFRQAPGQEREFVAGI | 1 |
| | GWSGGDTLYADSVRGRFTNSRDNSKNTLYLQMNSLRAEDTAVYYCAARQGQ | |
| | YIYSSMRSDSYDYWGQGTLVTVSS | |
| LCP0196 | EVQLVESGGGLVQPGGSLRLSCAASGRAFSDYAMAWFRQAPGQEREFVAGI | 276 |
| | GWSGGDTLYADSVRGRFTISKDNSKNTLYLQMNSLRAEDTAVYYCAARQGQ | |
| | YIYSSMRSDSYDYWGQGTLVTVSS | |
| LCP0197 | EVQLVESGGGLVQPGGSLRLSCAASGRAFSDYAMAWFRQAPGQEREFVAGI | 2 |
| | GWSGGDTLYADSVRGRFTISRDNAKNTLYLQMNSLRAEDTAVYYCAARQGQ | |
| | YIYSSMRSDSYDYWGQGTLVTVSS | |
| LCP0198 | EVQLVESGGGLVQPGGSLRLSCAASGRAFSDYAMAWFRQAPGQEREFVAGI | 277 |
| | GWSGGDTLYADSVRGRFTISRDNSKNRLYLQMNSLRAEDTAVYYCAARQGQ | |
| | YIYSSMRSDSYDYWGQGTLVTVSS | |
| LCP0199 | EVQLVESGGGLVQPGGSLRLSCAASGRAFSDYAMAWFRQAPGQEREFVAGI | 3 |
| | GWSGGDTLYADSVRGRFTISRDNSKNTMYLQMNSLRAEDTAVYYCAARQGQ | |
| | YIYSSMRSDSYDYWGQGTLVTVSS | |
| LCP0200 | EVQLVESGGGLVQPGGSLRLSCAASGRAFSDYAMAWFRQAPGQEREFVAGI | 278 |
| | GWSGGDTLYADSVRGRFTISRDNSKNTLSLQMNSLRAEDTAVYYCAARQGQ | |
| | YIYSSMRSDSYDYWGQGTLVTVSS | |
| LCP0201 | EVQLVESGGGLVQPGGSLRLSCAASGRAFSDYAMAWFRQAPGQEREFVAGI | 279 |
| | GWSGGDTLYADSVRGRFTISRDNSKNTLYLQMNSLKAEDTAVYYCAARQGQ | |
| | YIYSSMRSDSYDYWGQGTLVTVSS | |
| LCP0202 | EVQLVESGGGLVQPGGSLRLSCAASGRAFSDYAMAWFRQAPGQEREFVAGI | 280 |
| | GWSGGDTLYADSVRGRFTISRDNSKNTLYLQMNSLRPEDTAVYYCAARQGQ | |
| | YIYSSMRSDSYDYWGQGTLVTVSS | |
| LCP0203 | EVQLVESGGGLVQPGGSLRLSCAASGRAFSDYAMAWFRQAPGQGLEFVAGI | 4 |
| | GWSGGDTLYADSVRGRFTISRDNSKNTLYLQMNSLRAEDTAVYYCAARQGQ | |
| | YIYSSMRSDSYDYWGQGTLVTVSS | |

### Example 7. Isolation of VHH Domains Binding to Human Serum Albumin

Albumin is an abundant protein in serum and has sufficient molecular weight to avoid removal by filtration through the glomerular filtration barrier. Removal of albumin from serum by intracellular degradation is inhibited by the interaction of FcRn with albumin that occurs at low pH. This interaction results in trafficking of the albumin-FcRn complex back to the plasma membrane where albumin is released back into blood upon exposure to the more neutral pH of the blood.

### Overview of the Process for Generating Anti-HSA VHH

An immune biased VHH anti-HSA phage display library was produced from B cells of an immunized llama for anti-C5 VHH domains and for anti-HSA VHH domains. Upon obtaining endpoint titers greater than 1,000,000 towards HSA, PBMCs were harvested, RNA isolated and VHH regions genetically isolated. As described in detail for anti-C5 VHH domains in Examples 2-4, these anti-HSA VHH sequences were cloned into a pIII fusion phagemid, resulting in a library of 6×10⁸ independent clones.

Standard phage display panning techniques were used to select VHH domains reactive towards HSA and CSA (Cynomolgus monkey serum albumin). Outputs from three rounds of panning were analyzed by ELISA and Sanger sequencing. In parallel, next generation sequencing (NGS) was used to examine populations of sequences within the original library, or sequences that were enriched by panning. A total of ˜1000 clones were isolated and analyzed using these methods.
Llama immunization and VHH phage library construction. A llama was immunized with HSA. The primary boost consisted of 500 μg antigen mixed with complete Freunds adjuvant. Boost immunizations of 500 μg antigen in incomplete Freunds adjuvant were given at 2 weeks, 4 weeks, 8 weeks and 12 weeks. Sera titers were monitored with test bleeds approximately 2 weeks after each boost. Test bleeds were analyzed by ELISA to determine titer of immune response. An anti-HSA sera titer was detected at 20× signal above the pre-bleed for the 1:100,000 dilution, therefore a production bleed of 500 mL was processed to obtain ˜7×10⁸ PBMCs for RNA isolation and library production. Total RNA from PBMCs was purified with phenol/chloroform extraction, followed by a silica-spin column, and total RNA was eluted with RNase free water. Quality of RNA was evaluated by determining the OD_{260/280} ratio and by agarose gel electrophoresis. cDNA was synthesized using llama heavy chain specific reverse primers. VHH (heavy chain only) fragments were separated from VH (conventional heavy chain) fragments via gel electrophoresis.

The VHH fragments were modified with Sfil sites and cloned into pADL-10b, and the DNA library was transformed into TG1 cells. A total of 6×10⁸ independent clones were obtained for the library. All clones were harvested and stored in 25% glycerol at −80C until use. Library quality was validated by analysis of 105 clones for the presence of an insert with a correct reading frame, uniqueness, and presence of primer sequences.
Phage display panning and screening. An aliquot of the anti-HSA VHH library glycerol stock comprising 3.75×101⁰ cells was cultured in 2×YT media supplemented with 2% glucose and 100 μg/mL carbenicillin. Cells were grown at 37C with shaking at ˜250 rpm until and an OD₆₀₀ of ˜0.6 was obtained. Helper phage was added at a multiplicity of infection (MOI) of 20 and the culture was incubated for 30 minutes without shaking, followed by incubation for 30 minutes with shaking at 37C. Cells were harvested and resuspended in 2×YT media supplemented with 25 μg/mL Carbenicillin, 50 μg/mL kanamycin, and 200 μM IPTG. Cultures were shaken overnight at 30C and 250 rpm. Media was clarified by centrifugation, phage were precipitated by addition of ¼th volume of 10% PEG-8000/2.5 M NaCl and incubation on ice for 30 minutes. Phage were pelleted by centrifugation at 7500 rpm for 15 minutes at 4C in an SLA3000 rotor. The pellet was resuspended in Superblock (Thermo Scientific, 37515).

An aliquot of phage was deselected with M280 Streptavidin beads (Life Technologies, 11205D) for 30 minutes at room temperature, the beads were removed using a magnet, and phage-containing supernatant was transferred to a new Eppendorf tube. Phage were supplemented with 10 μg of biotinylated HSA, incubated with rotation at room temperature for 30 minutes, and then supplemented with M280 streptavidin beads to immobilize biotinylated HSA. Beads were washed 11 times with PBS/0.05%

Tween wash buffer, eluted with 0.1 M glycine, pH 2.7, and then the elution buffer was neutralized with 1 M Tris, pH 9.0. Eluted phage were rescued into log phage TG1 cells and outgrowths recovered on 250 cm×250 cm LB Carbenicillin, 2% glucose trays. Titers were determined by serial dilution of an aliquot of the phage rescue. A second round of panning was performed essentially as described above, using an aliquot of the round one outgrowth and 5 μg of biotinylated HSA for selections.

To screen clones for reactivity to HSA, individual clones were picked into 96 well plates, cultured in a volume of 250 μL 2×YT supplemented with 100 μg/mL Carbenicillin and 2% glucose overnight at 37C. Each well was subcultured by transfer of 5 μL dense overnight culture into 250 μL fresh media. An aliquot was submitted for rolling circle amplification sequence analysis to determine the encoded insert. Cells were grown to an OD₆₀₀ of ˜0.6, then supplemented with M13 helper phage at an MOI of 20 for one hour. Cells were harvested by centrifugation and media replaced with 250 μL per well of 2×YT supplemented with 100 μg/mL Carbenicillin and 50 μg/mL kanamycin. Plates were then incubated overnight at 30C with shaking at 250 rpm. Media was clarified by centrifugation to prepare phage supernatants for use in ELISA assays.

For ELISA analysis, streptavidin-coated, pre-blocked 96-well plates (Pierce, 15500) were incubated with has-Biotin at 2 μg/mL for 30 minutes at room temperature with shaking. Plates were washed and then blocking was repeated for 1 hour at room temperature. Plates were again washed and supplemented with 50 μL of clarified supernatant for 30 minutes at room temperature. Plates were washed three times, then incubated with anti-M13 HRP antibody (GE Healthcare, Cat #27-9421-01) in blocking buffer for 30 minutes at room temperature. Plates were washed four times, then supplemented with 1-step Ultra TMB-ELISA reagent (Thermo Scientific, Cat #34029), color developed, and the reaction stopped using 2 M sulfuric acid stop solutions. OD₄₅₀ readings were determined using a BioRad iMark plate reader.

NGS was used to examine populations of sequences within the original library, or sequences that were enriched by panning. For NGS, phagemid DNA was isolated from outgrowths of the initial library, round 1 panning, and round 2 panning. The VHH cassette was released from the phagemid by restriction digestion, VHH encoding bands isolated by agarose gel electrophoresis, and DNA purified using DNA affinity columns. This DNA was submitted for library production and analysis on the MiSeq 2×300 platform.

### Example 8. Expression and Purification of VHH Domains Binding to HSA

VHH sequences selected using the above methodologies were synthesized with N-terminal signal peptides and C-terminal 6× His-tags (SEQ ID NO: 324) and cloned into a mammalian expression construct. The published MSA21 VHH domain (International Publication No. WO 2004/062551 A2) and genetically modified versions of individual clones (deglycosylated or humanized) were prepared by synthesis of GeneBlocks (Integrated DNA Technologies) and infusion cloning into a standard mammalian expression vector. These constructs were transfected into 293expi cells and supernatant harvested at 96 hours post-transfection. Supernatants were dialyzed against PBS and VHH-His proteins purified using standard chromatography methods. Purified proteins were buffer exchanged into PBS and quantified using OD and extinction coefficient.

### Example 9. Characterization of Immobilized VHH Domains Binding to Soluble HSA, CSA and Mouse Serum Albumin

Mammalian expression vectors were created for 112 VHH sequences and protein produced in the 293 expi expression system. VHH sequences were first analyzed by SDS-PAGE and Coomassie staining to determine approximate concentration relative to a known standard. Supernatant concentrations were then normalized and subjected to biolayer interferometry on an Octet HTX (Pall/ForteBio). Penta-His sensors were exposed to kinetics buffer for 60 seconds to establish baseline measurements. The sensors were then loaded with VHH-His containing supernatants for 300 seconds before a second baseline was established in kinetics buffer over 120 seconds. Tips were then incubated with 100 nM HSA or CSA in kinetics buffer for 600 seconds and dissociation measured over an additional 600 seconds.

Of the 112 VHH domains analyzed, 12 domains demonstrated binding to biotinylated HSA and three clones (HAS040, HAS041 and HAS042) interacted with both biotinylated CSA and biotinylated HSA. The sequences of these 12 anti-HSA VHH domains, including one or more humanized versions thereof, are shown in Table 6, with the CDRs of these anti-HSA VHH domains shown in Table 7.

TABLE 6
Sequences for anti-albumin VHH domains

| VHH domain | Sequence | SEQ ID NO: |
|---|---|---|
| HAS020 | QVQLVESGGGLVQAGGSLRLSCAASGRTFGSDAAGWFRQASGK | 22 |
| | EREFVASISWSGGYTYYADSVKGRFTISSDNVKNTVYLQMNSL | |
| | TPEDTAVYFCATGNRYSDYRISLVTPSQYEYWGQGTLVTVS | |
| HAS038 | QVQLVESGGGLVQPGGSLRLSCTGSGHSFSTYTVGWFRQAPGE | 23 |
| | ERKFVASISWSGEVTLYGDSVKGRFTISRDNRKKTVYLQMHSL | |
| | KPEDSAIYYCAAKRGGRPTDSSDDYFYWGQGTQVTVSS | |
| HAS040 | QVQLNESGGGMVQAGGSLRLSCAASGRTVSNYAAGWFRQAPGK | 24 |
| | EREFVAAINWNKTTTYADSVKGRFIISREYAKNTVALQMNSLK | |
| | PEDTAVYYCAAVFRIVAPKTQYEYDYWGQGTQVTVSS | |
| HAS041 | QVQLIESGGGLVQAGGSLGLSCAASGRPVSNYAAAWFRQAPGK | 25 |
| | EREFVAAINWNKTATYADSVKGRFTISRDNAKSTVALQMNSLK | |
| | PEDTAVYYCAAVFRVVAPKTQYDYDYWGQGTQVTVSS | |
| HAS042 | EVQLVESGGGLVKPGGSLRLSCAASGRPVSNYAAAWFRQAPGK | 26 |
| | EREFVSAINWQKTATYADSVKGRFTISRDNAKNSLYLQMNSLR | |
| | AEDTAVYYCAAVFRVVAPKTQYDYDYWGQGTLVTVSS | |
| HAS044 | QVQLVESGGGLVQAGGSLRLSCAASGRTFSSYAIGWFRQAPGK | 27 |
| | AREFVARVSTIAGDTDYADSVKGRFTISRDNAKNTVYLQMNSL | |
| | KPEDTAVYYCAADSYNVRLVTGEADYWGEGTQVTVSS | |
| HAS077 | QVQLVESGGGLVQAGGSLRLSCAASGRTFSSYAIGWFRQAPGK | 28 |
| | AREFVARVSTIAGDTDYADSVKGRFTISRDNAKNTVYLQMNSL | |
| | KPEDTAVYYCAADSYNVRLGTGEADYWGEGTQVTVSS | |
| HAS079 | EVQLVESGGGLVQAGDSLRLSCAASGFTFSNYAIGWFRQAPGK | 29 |
| | AREFVARVSTIAGDTDYANAVKGRFTISRDNAKNTVYLQMNSL | |
| | KPDDTAVYYCAAESYNVRLVTGEADYWGEGTQVTVSS | |
| HAS080 | QVRLAESGGGRVQAGESLRLSCVASGRTFSNDAAGWFREASGK | 30 |
| | EREFVASISWSGNYTYYADSVKGRFTISEDNVKNTVYLQMTSL | |
| | KPEDTAVYYCAAGNRYSDYRISLVTPRLYEYWGQGTQVTVS | |
| HAS081 | QVQLVESGGGLVQAGGSLRLSCAASGRTFSSDAAGWFRQASGK | 31 |
| | EREFVAAISWSGNYTYSADSVKGRFTISSDNVKNTVYLQMNSL | |
| | KPEDTAVYLCAAGNRYSDYRISLVTPSQYEYWGQGTQVTVS | |
| HAS091 | QVQLVESGGGLVQAGGSLRLSCAASGRTFGSDAAGWFRQASGK | 32 |
| | EREFVASISWSGGYTYYADSGTGRFTISSDNVKNTVYLQMNSL | |
| | TPEDTAVYFCATGNRDSDYRISLVTPSQYEYWGQGTQVTVS | |
| HAS093 | QVQLVESGGGLVQAGGSLRLSCAASGRTFGSDAAGWFRQASGK | 33 |
| | EREFVASISWSGGYTYYADSGKGRFTISSDNVKNTVYLQMNSL | |
| | TPEDTAVYFCATGNRYSDYRISLVTPSQYDYWGQGTQVTVS | |
| HAS096 | QVQLVESGGGLVQAGGSLRLSCAASGRTFGSDAAGWFRQASGK | 34 |
| | EREFVASISWSGGYTYYADSVKGRFTSSSDNVKNTVYLQMNSL | |
| | TPEDTAVYFCATVNRYSDYRISLVTPSQYEYWGQGTQVTVS | |

TABLE 7
CDR sequences for anti-albumin VHH domains.

| | CDR1 sequence | CDR2 sequence | CDR3 sequence |
|---|---|---|---|
| VHH domain | [SEQ ID NO:] | [SEQ ID NO] | [SEQ ID NO:] |
| HAS020 | GRTFGSDA [35] | ISWSGGYT [44] | ATGNRYSDYRISLVTPSQYEY [52] |
| HAS038 | GHSFSTYT [36] | ISWSGEVT [45] | AAKRGGRPTDSSDDYFY [53] |
| HAS040 | GRTVSNYA [37] | INWNKTTT [46] | AAVFRIVAPKTQYEYDY [54] |
| HAS041 | GRPVSNYA [38] | INWNKTAT [47] | AAVFRVVAPKTQYDYDY [55] |
| HAS042 | GRPVSNYA [38] | INWQKTAT [48] | AAVFRVVAPKTQYDYDY [55] |
| HAS044 | GRTFSSYA [39] | VSTIAGDT [49] | AADSYNVRLVTGEADY [56] |
| HAS077 | GRTFSSYA [39] | VSTIAGDT [49] | AADSYNVRLGTGEADY [57] |
| HAS079 | GFTFSNYA [40] | VSTIAGDT [49] | AAESYNVRLVTGEADY [58] |
| HAS080 | GRTFSNDA [41] | ISWSGNYT [50] | AAGNRYSDYRISLVTPRLYEY [59] |
| HAS081 | GRTFSSDA [42] | ISWSGNYT [50] | AAGNRYSDYRISLVTPSQYEY [60] |
| HAS091 | GRTFGSDA [43] | ISWSGGYT [51] | ATGNRDSDYRISLVTPSQYEY [61] |
| HAS093 | GRTFGSDA [43] | ISWSGGYT [51] | ATGNRYSDYRISLVTPSQYDY [62] |
| HAS096 | GRTFGSDA [43] | ISWSGGYT [51] | ATVNRYSDYRISLVTPSQYEY [63] |

### Example 10. Characterization of Albumin-Binding Kinetics by Biacore

The binding kinetics of the VHH domains HAS040 and HAS041 to HSA or CSA were determined using SPR on a Biacore 3000 instrument. Biotinylated albumin was captured onto a CAP chip saturated with Biotin CAPture reagent containing deoxyribooligonucleotides (obtained from GE Healthcare). Concentrations of purified VHH domains were injected for 5 minutes at a flowrate of 50 μL/min. Three concentrations were assessed per VHH domain. Bound analyte was allowed to dissociate for 600 seconds. The chip surface was regenerated after each concentration by injecting 6 M guanidine HCl/0.25 M NaOH for 2 minutes at 10 μL/min. Kinetics were determined at pH 7.4 and pH 6.0 in HBS-EP buffer using a 1:1 Langmuir model (local Rₘₐₓ and constant RI) and double reference subtraction (subtraction of a buffer concentration cycle from the sample concentration cycle and subtraction of a parallel reference flow cell). The MSA21 VHH domain (International Publication No. WO 2004/062551 A2) (sequence:

(SEQ ID NO: 322)
LEQVQLQESGGGLVQPGGSLRLSCEASGFTFSRFGMTWVRQAPGKGVEW
VSGISSLGDSTLYADSVKGRFTISRDNAKNTLYLQMNSLKPEDTAVYYC
TIGGSLNPGGQGTQVTVSS

was prepared and used as a comparator in these assays.

The results of this assay are shown in Table 8. Binding affinities were observed in the 0.3-5 nM range, indicating that the HAS040 and HAS041 domains have sufficient affinity at both pH 6 and pH 7.4 to facilitate half-life extension. Furthermore, these VHH domains demonstrated binding to CSA and HSA with very similar affinities, strengthening the predictive nature of half-life extension studies to be performed in primates.

TABLE 8
Results of Biacore characterization of anti-albumin VHH domains.

| | | kₐ | k_{d} | K_{D} | |
|---|---|---|---|---|---|
| Sample | Albumin/pH | (1/Ms) | (1/s) | (M) | Chi² |
| HAS40 | CSA/pH 6.0 | 3.68E+05 | 2.81E−04 | 7.64E−10 | 0.05 |
| | CSA/pH 7.4 | 1.04E+06 | 5.62E−04 | 5.39E−10 | 0.1 |
| | HSA/pH 6.0 | 4.45E+05 | 2.08E−04 | 4.66E−10 | 0.09 |
| | HSA/pH 7.4 | 1.29E+06 | 4.40E−04 | 3.41E−10 | 0.03 |
| HAS41 | CSA/pH 6.0 | 3.12E+05 | 7.39E−04 | 2.37E−09 | 0.41 |
| | CSA/pH 7.4 | 1.07E+06 | 1.23E−03 | 1.15E−09 | 0.18 |
| | HSA/pH 6.0 | 3.73E+05 | 3.87E−04 | 1.04E−09 | 0.12 |
| | HSA/pH 7.4 | 1.23E+06 | 5.66E−04 | 4.61E−10 | 0.03 |
| MSA21 | CSA/pH 6.0 | 2.80E+05 | 1.53E−03 | 5.47E−09 | 0.05 |
| | CSA/pH 7.4 | 5.61E+05 | 2.16E−03 | 3.85E−09 | 0.05 |
| | HSA/pH 6.0 | 3.30E+05 | 1.81E−03 | 5.46E−09 | 0.06 |
| | HSA/pH 7.4 | 1.13E+06 | 3.93E−03 | 3.49E−09 | 0.07 |

### Example 11. Demonstration of Non-Competitive Albumin Binding by VHH and FcRn

Recycling of albumin from endocytic vesicles is mediated by interaction with FcRn. It was, therefore, important to determine whether the VHH would interfere with the interaction of HSA and FcRn. To determine whether the HAS040 and HAS041 VHH domains bind to the same epitope as FcRn, the binding of FcRn to HSA that had been saturated with anti-HSA VHH domains was analyzed on a Biacore 3000 instrument at pH 6.0 in HBS-EP buffer. HSA was directly immobilized onto a CM5 chip to reach a target density of 250 RUs (resonance units) using amine coupling. VHH domains were diluted to approximately 1-10 μg/mL and injected to achieve saturation (3 minutes at 50 μL/min). One concentration of FcRn was injected over the HSA: VHH surface to obtain kinetics for 5 minutes at 50 μL/min. Dissociation was allowed for 180 seconds before regeneration. The chip surface was regenerated by injecting 20 μL of 25 mM NaOH at 100 L/min. Kinetics were determined using a 1:1 Langmuir model (local Rₘₐₓ and constant RI) and double reference subtraction (subtraction of a buffer concentration cycle from the sample concentration cycle and subtraction of a parallel reference flow cell).

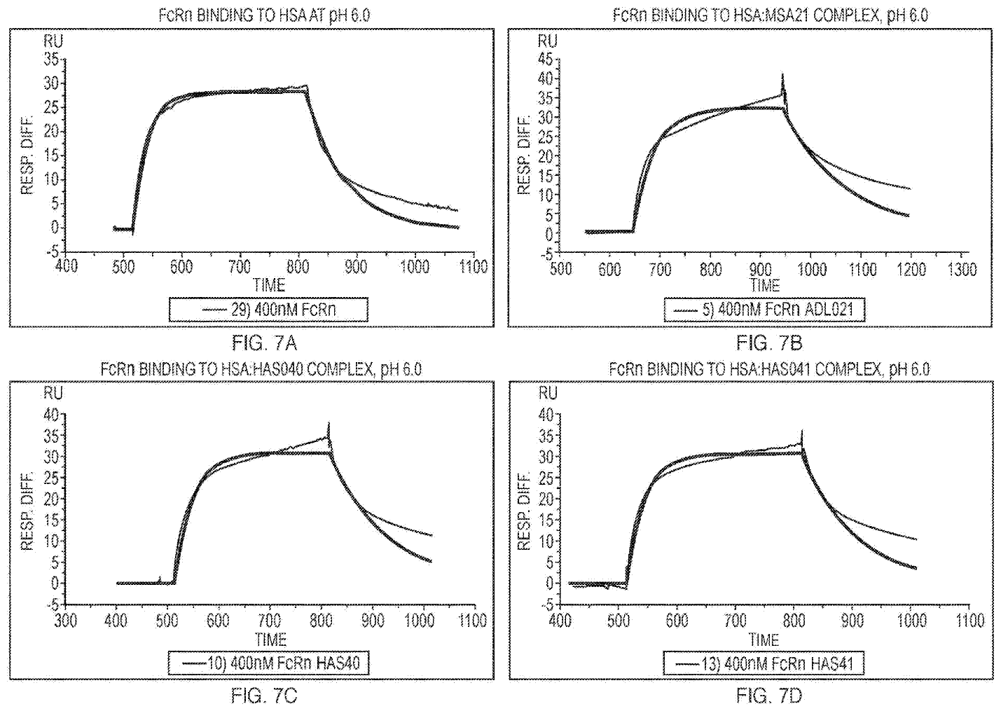

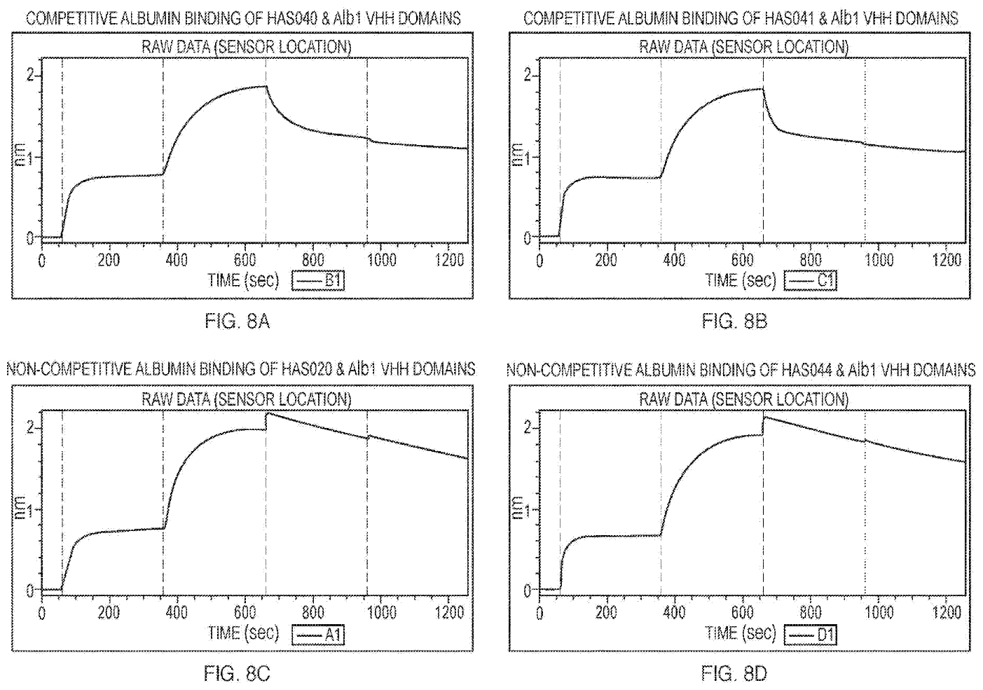

Results are shown in FIG. 7. In FIG. 7A, the direct interaction of FcRn with an HSA saturated surface resulted in a response difference of 30 RUs. Similar RUs were obtained when 400 nM FcRn was injected over surfaced saturated with complexes of HSA with MSA21 (ADL021) (FIG. 7B), HAS040 (FIG. 7C) or HAS041 (FIG. 7D). Based on these data, HAS040 and HAS041 do not to interfere with FcRn binding and are expected to be recycled from the endosome via the interaction of albumin with FcRn.

### Example 12. Generation of Anti-C5 and Anti-Albumin Bispecific Fusion Proteins

Anti-C5 VHH domains were fused to an anti-albumin domain to generate bispecific molecules. Four different linker lengths (G₄S)₃ (SEQ ID NO: 106), (G₄S)₄ (SEQ ID NO: 107), (G₄S)₅ (SEQ ID NO: 108) and (G₄S)₆ (SEQ ID NO: 109), and two different orientations (N-terminal or C-terminal) of anti-albumin domain were evaluated. Constructs were expressed in HEK293F cells and purified using Protein A affinity chromatography. Purified fusion molecules were evaluated in Biacore experiments. Human C5 was biotinylated and immobilized on a biacore chip, purified bispecific molecules were injected to saturate the chip followed by three different concentrations of human serum albumin to obtain kinetics. Measured affinity to human serum albumin was used as a proxy to compare the different linker lengths. (G₄S)₃ (SEQ ID NO: 106) was chosen as the optimal linker length to generate bispecific fusions. N-terminal or C-terminal anti-albumin fusions were also evaluated in the same experiment. Different orientations were found to be optimal for different anti-C5 VHH domains. The N-versus C-terminal orientation of the constructs is specified below the construct name in Table 9 with (C5/HSA) indicating the anti-C5 domain is located N-terminal to the anti-HSA domain. Likewise, with (HSA/C5) indicates the anti-HSA domain is located N-terminal to the anti-C5 domain.

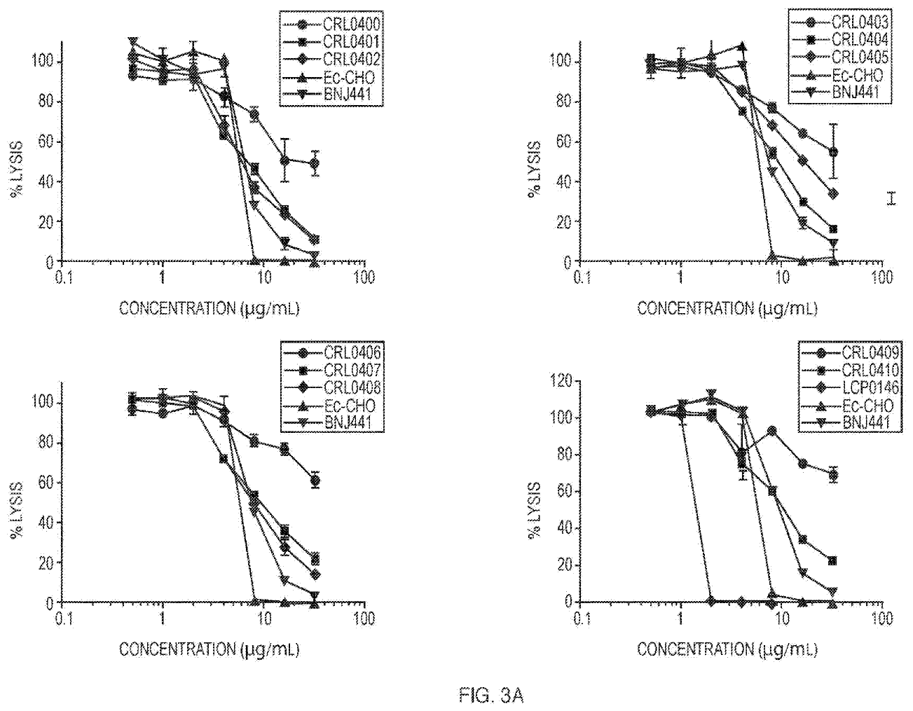

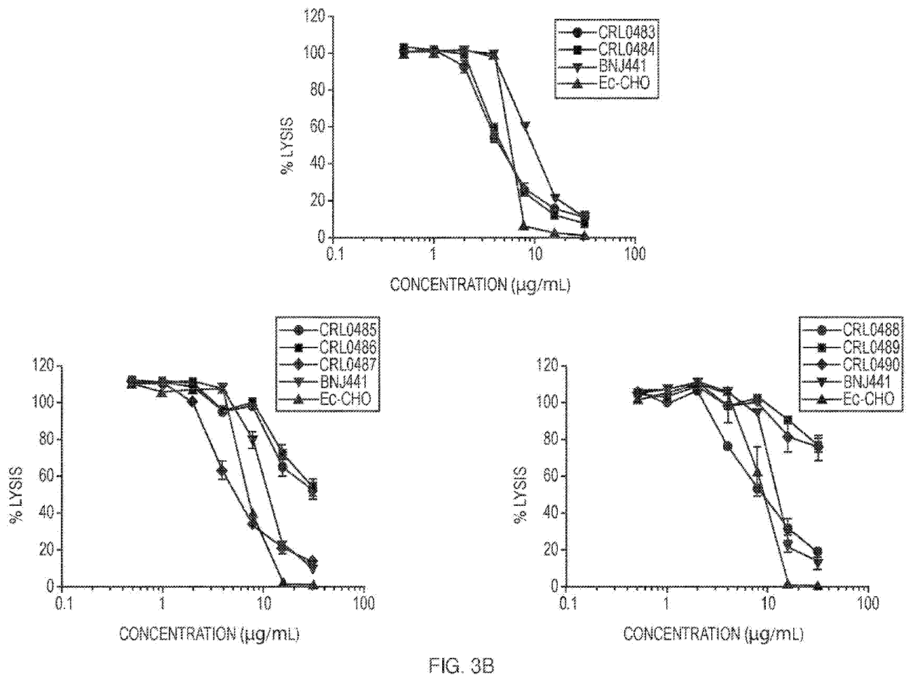

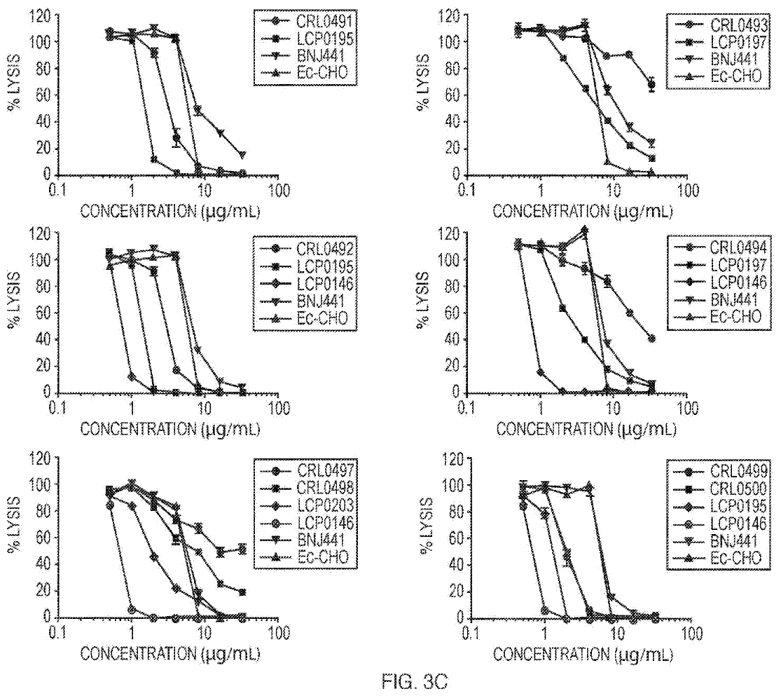

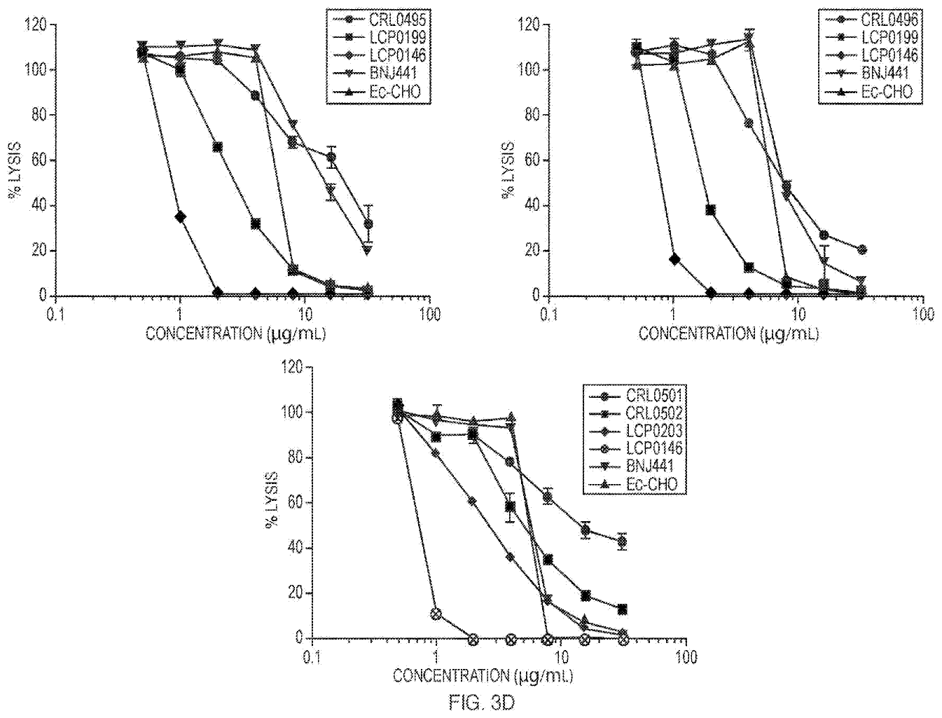

After selecting the optimal linker length, a series of different bispecific fusion molecules were generated with humanized anti-C5 VHH domains fused to two different anti-albumin domains (shown in Table 8). These constructs were expressed in Expi293 cells and purified using Protein A chromatography. Purified bispecific fusion proteins were tested in hemolysis assays and the results are shown in FIGS. 3A and 3B.

TABLE 9
Anti-C5/Anti-Albumin Fusion Proteins

| Name | Sequence | SEQ ID NO: |
|---|---|---|
| CRL0400 | EVQLLESGGGLVQPGGSLRLSCAASGFTFRSFGMSWVRQAPGKGPE | 64 |
| (HSA/C5) | WVSSISGSGSDTLYADSVKGRFTISRDNSKNTLYLQMNSLRPEDTA | |
| | VYYCTIGGSLSRSSQGTLVTVSSGGGGSGGGGSGGGGSEVQLVESG | |
| | GGLVQPGGSLRLSCAASGRHFSDYAMAWFRQAPGQEREFVAGIGWS | |
| | GGDTLYADSVRGRFTISRDNAKNTLYLQMNSLRAEDTAVYYCAARQ | |
| | GQYIYSSMRSDSYDYWGQGTLVTVSS | |
| CRL0401 | EVQLLESGGGLVQPGGSLRLSCAASGFTFRSFGMSWVRQAPGKGPE | 65 |
| (HSA/C5) | WVSSISGSGSDTLYADSVKGRFTISRDNSKNTLYLQMNSLRPEDTA | |
| | VYYCTIGGSLSRSSQGTLVTVSSGGGGSGGGGSGGGGSEVQLVESG | |
| | GGLVQPGGSLRLSCAASGRAHSDYAMAWFRQAPGQEREFVAGIGWS | |
| | GGDTLYADSVRGRFTISRDNAKNTLYLQMNSLRAEDTAVYYCAARQ | |
| | GQYIYSSMRSDSYDYWGQGTLVTVSS | |
| CRL0402 | EVQLLESGGGLVQPGGSLRLSCAASGFTFRSFGMSWVRQAPGKGPE | 66 |
| (HSA/C5) | WVSSISGSGSDTLYADSVKGRFTISRDNSKNTLYLQMNSLRPEDTA | |
| | VYYCTIGGSLSRSSQGTLVTVSSGGGGSGGGGSGGGGSEVQLVESG | |
| | GGLVQPGGSLRLSCAASGRHHSDYAMAWFRQAPGQEREFVAGIGWS | |
| | GGDTLYADSVRGRFTISRDNAKNTLYLQMNSLRAEDTAVYYCAARQ | |
| | GQYIYSSMRSDSYDYWGQGTLVTVSS | |
| CRL0403 | EVQLLESGGGLVQPGGSLRLSCAASGFTFRSFGMSWVRQAPGKGPE | 67 |
| (HSA/C5) | WVSSISGSGSDTLYADSVKGRFTISRDNSKNTLYLQMNSLRPEDTA | |
| | VYYCTIGGSLSRSSQGTLVTVSSGGGGSGGGGSGGGGSEVQLVESG | |
| | GGLVQPGGSLRLSCAASGRHFSDYAMAWFRQAPGQEREFVAGIGWS | |
| | GGDTLYADSVRGRFTISRDNSKNTMYLQMNSLRAEDTAVYYCAARQ | |
| | GQYIYSSMRSDSYDYWGQGTLVTVSS | |
| CRL0404 | EVQLLESGGGLVQPGGSLRLSCAASGFTFRSFGMSWVRQAPGKGPE | 68 |
| (HSA/C5) | WVSSISGSGSDTLYADSVKGRFTISRDNSKNTLYLQMNSLRPEDTA | |
| | VYYCTIGGSLSRSSQGTLVTVSSGGGGSGGGGSGGGGSEVQLVESG | |
| | GGLVQPGGSLRLSCAASGRAHSDYAMAWFRQAPGQEREFVAGIGWS | |
| | GGDTLYADSVRGRFTISRDNSKNTMYLQMNSLRAEDTAVYYCAARQ | |
| | GQYIYSSMRSDSYDYWGQGTLVTVSS | |
| CRL0405 | EVQLLESGGGLVQPGGSLRLSCAASGFTFRSFGMSWVRQAPGKGPE | 69 |
| (HSA/C5) | WVSSISGSGSDTLYADSVKGRFTISRDNSKNTLYLQMNSLRPEDTA | |
| | VYYCTIGGSLSRSSQGTLVTVSSGGGGSGGGGSGGGGSEVQLVESG | |
| | GGLVQPGGSLRLSCAASGRHHSDYAMAWFRQAPGQEREFVAGIGWS | |
| | GGDTLYADSVRGRFTISRDNSKNTMYLQMNSLRAEDTAVYYCAARQ | |
| | GQYIYSSMRSDSYDYWGQGTLVTVSS | |
| CRL0406 | EVQLVESGGGLVKPGGSLRLSCAASGRPVSNYAAAWFRQAPGKERE | 70 |
| (HSA/C5) | FVSAINWQKTATYADSVKGRFTISRDNAKNSLYLQMNSLRAEDTAV | |
| | YYCAAVFRVVAPKTQYDYDYWGQGTLVTVSSGGGGSGGGGSGGGGS | |
| | EVQLVESGGGLVQPGGSLRLSCAASGRHFSDYAMAWFRQAPGQERE | |
| | FVAGIGWSGGDTLYADSVRGRFTISRDNAKNTLYLQMNSLRAEDTA | |
| | VYYCAARQGQYIYSSMRSDSYDYWGQGTLVTVSS | |
| CRL0407 | EVQLVESGGGLVKPGGSLRLSCAASGRPVSNYAAAWFRQAPGKERE | 71 |
| (HSA/C5) | FVSAINWQKTATYADSVKGRFTISRDNAKNSLYLQMNSLRAEDTAV | |
| | YYCAAVFRVVAPKTQYDYDYWGQGTLVTVSSGGGGSGGGGSGGGGS | |
| | EVQLVESGGGLVQPGGSLRLSCAASGRAHSDYAMAWFRQAPGQERE | |
| | FVAGIGWSGGDTLYADSVRGRFTISRDNAKNTLYLQMNSLRAEDTA | |
| | VYYCAARQGQYIYSSMRSDSYDYWGQGTLVTVSS | |
| CRL0408 | EVQLVESGGGLVKPGGSLRLSCAASGRPVSNYAAAWFRQAPGKERE | 72 |
| (HSA/C5) | FVSAINWQKTATYADSVKGRFTISRDNAKNSLYLQMNSLRAEDTAV | |
| | YYCAAVFRVVAPKTQYDYDYWGQGTLVTVSSGGGGSGGGGSGGGGS | |
| | EVQLVESGGGLVQPGGSLRLSCAASGRHHSDYAMAWFRQAPGQERE | |
| | FVAGIGWSGGDTLYADSVRGRFTISRDNAKNTLYLQMNSLRAEDTA | |
| | VYYCAARQGQYIYSSMRSDSYDYWGQGTLVTVSS | |
| CRL0409 | EVQLVESGGGLVKPGGSLRLSCAASGRPVSNYAAAWFRQAPGKERE | 73 |
| (HSA/C5) | FVSAINWQKTATYADSVKGRFTISRDNAKNSLYLQMNSLRAEDTAV | |
| | YYCAAVFRVVAPKTQYDYDYWGQGTLVTVSSGGGGSGGGGSGGGGS | |
| | EVQLVESGGGLVQPGGSLRLSCAASGRHFSDYAMAWFRQAPGQERE | |
| | FVAGIGWSGGDTLYADSVRGRFTISRDNSKNTMYLQMNSLRAEDTA | |
| | VYYCAARQGQYIYSSMRSDSYDYWGQGTLVTVSS | |
| CRL0410 | EVQLVESGGGLVKPGGSLRLSCAASGRPVSNYAAAWFRQAPGKERE | 74 |
| (HSA/C5) | FVSAINWQKTATYADSVKGRFTISRDNAKNSLYLQMNSLRAEDTAV | |
| | YYCAAVFRVVAPKTQYDYDYWGQGTLVTVSSGGGGSGGGGSGGGGS | |
| | EVQLVESGGGLVQPGGSLRLSCAASGRAHSDYAMAWFRQAPGQERE | |
| | FVAGIGWSGGDTLYADSVRGRFTISRDNSKNTMYLQMNSLRAEDTA | |
| | VYYCAARQGQYIYSSMRSDSYDYWGQGTLVTVSS | |
| CRL0411 | EVQLVESGGGLVKPGGSLRLSCAASGRPVSNYAAAWFRQAPGKERE | 75 |
| (HSA/C5) | FVSAINWQKTATYADSVKGRFTISRDNAKNSLYLQMNSLRAEDTAV | |
| | YYCAAVFRVVAPKTQYDYDYWGQGTLVTVSSGGGGSGGGGSGGGGS | |
| | EVQLVESGGGLVQPGGSLRLSCAASGRHHSDYAMAWFRQAPGQERE | |
| | FVAGIGWSGGDTLYADSVRGRFTISRDNSKNTMYLQMNSLRAEDTA | |
| | VYYCAARQGQYIYSSMRSDSYDYWGQGTLVTVSS | |
| CRL0483 | EVQLVESGGGLVQPGGSLRLSCAASGRTFSGILSPYAVGWFRQAPG | 76 |
| (C5/HSA) | KGLEFVSTITSGGSAIYTDSVKGRFTISRDNAKDSLYLQMNSLRAE | |
| | DTAVYYCAVRTRRYGSNLGEVPQENEYGYWGQGTLVTVSSGGGGSG | |
| | GGGSGGGGSEVQLLESGGGLVQPGGSLRLSCAASGFTFRSFGMSWV | |
| | RQAPGKGPEWVSSISGSGSDTLYADSVKGRFTISRDNSKNTLYLQM | |
| | NSLRPEDTAVYYCTIGGSLSRSSQGTLVTVSS | |
| CRL0484 | EVQLVESGGGLVQPGGSLRLSCAASGRTFSGILSPYAVGWFRQAPG | 77 |
| (C5/HSA) | KGLEFVSTITSGGSAIYTDSVKGRFTISRDNAKDSLYLQMNSLRAE | |
| | DTAVYYCAVRTRRYGSNLGEVPQENEYGYWGQGTLVTVSSGGGGSG | |
| | GGGSGGGGSEVQLVESGGGLVKPGGSLRLSCAASGRPVSNYAAAWF | |
| | RQAPGKEREFVSAINWQKTATYADSVKGRFTISRDNAKNSLYLQMN | |
| | SLRAEDTAVYYCAAVFRVVAPKTQYDYDYWGQGTLVTVSS | |
| CRL0485 | EVQLVESGGGLVQPGGSLRLSCAASGRTFSGILSPYAVGWFRQAPG | 78 |
| (C5/HSA) | KGLEFVSTITSGGSAIYTDSVKGRFTISRDNAKNTLYLQMNSLRAE | |
| | DTAVYYCAVRTRRYGSNLGEVPQENEYGYWGQGTLVTVSSGGGGSG | |
| | GGGSGGGGSEVQLLESGGGLVQPGGSLRLSCAASGFTFRSFGMSWV | |
| | RQAPGKGPEWVSSISGSGSDTLYADSVKGRFTISRDNSKNTLYLQM | |
| | NSLRPEDTAVYYCTIGGSLSRSSQGTLVTVSS | |
| CRL0486 | EVQLVESGGGLVQPGGSLRLSCAASGRTFSGILSPYAVGWFRQAPG | 79 |
| (C5/HSA) | KGLEFVSTITSGGSAIYTDSVKGRFTISRDNAKNTLYLQMNSLRAE | |
| | DTAVYYCAVRTRRYGSNLGEVPQENEYGYWGQGTLVTVSSGGGGSG | |
| | GGGSGGGGSEVQLVESGGGLVKPGGSLRLSCAASGRPVSNYAAAWF | |
| | RQAPGKEREFVSAINWQKTATYADSVKGRFTISRDNAKNSLYLQMN | |
| | SLRAEDTAVYYCAAVFRVVAPKTQYDYDYWGQGTLVTVSS | |
| CRL0487 | EVQLVESGGGLVQPGGSLRLSCAASGRTFSGILSPYAVGWFRQAPG | 80 |
| (C5/HSA) | KGLEFVSTITSGGSAIYTDSVKGRFTISRDNAKNSVYLQMNSLRAE | |
| | DTAVYYCAVRTRRYGSNLGEVPQENEYGYWGQGTLVTVSSGGGGSG | |
| | GGGSGGGGSEVQLLESGGGLVQPGGSLRLSCAASGFTFRSFGMSWV | |
| | RQAPGKGPEWVSSISGSGSDTLYADSVKGRFTISRDNSKNTLYLQM | |
| | NSLRPEDTAVYYCTIGGSLSRSSQGTLVTVSS | |
| CRL0488 | EVQLVESGGGLVQPGGSLRLSCAASGRTFSGILSPYAVGWFRQAPG | 81 |
| (C5/HSA) | KGLEFVSTITSGGSAIYTDSVKGRFTISRDNAKNSVYLQMNSLRAE | |
| | DTAVYYCAVRTRRYGSNLGEVPQENEYGYWGQGTLVTVSSGGGGSG | |
| | GGGSGGGGSEVQLVESGGGLVKPGGSLRLSCAASGRPVSNYAAAWF | |
| | RQAPGKEREFVSAINWQKTATYADSVKGRFTISRDNAKNSLYLQMN | |
| | SLRAEDTAVYYCAAVFRVVAPKTQYDYDYWGQGTLVTVSS | |
| CRL0489 | EVQLVESGGGLVQPGGSLRLSCAASGRTFSGILSPYAVGWFRQAPG | 82 |
| (C5/HSA) | QGLEFVATITSGGSAIYTDSVKGRFTISRDNSKNTLYLQMNSLRAE | |
| | DTAVYYCAVRTRRYGSNLGEVPQENEYGYWGQGTLVTVSSGGGGSG | |
| | GGGSGGGGSEVQLLESGGGLVQPGGSLRLSCAASGFTFRSFGMSWV | |
| | RQAPGKGPEWVSSISGSGSDTLYADSVKGRFTISRDNSKNTLYLQM | |
| | NSLRPEDTAVYYCTIGGSLSRSSQGTLVTVSS | |
| CRL0490 | EVQLVESGGGLVQPGGSLRLSCAASGRTFSGILSPYAVGWFRQAPG | 83 |
| (C5/HSA) | QGLEFVATITSGGSAIYTDSVKGRFTISRDNSKNTLYLQMNSLRAE | |
| | DTAVYYCAVRTRRYGSNLGEVPQENEYGYWGQGTLVTVSSGGGGSG | |
| | GGGSGGGGSEVQLVESGGGLVKPGGSLRLSCAASGRPVSNYAAAWF | |
| | RQAPGKEREFVSAINWQKTATYADSVKGRFTISRDNAKNSLYLQMN | |
| | SLRAEDTAVYYCAAVFRVVAPKTQYDYDYWGQGTLVTVSS | |
| CRL0491 | EVQLLESGGGLVQPGGSLRLSCAASGFTFRSFGMSWVRQAPGKGPE | 84 |
| (C5/HSA) | WVSSISGSGSDTLYADSVKGRFTISRDNSKNTLYLQMNSLRPEDTA | |
| | VYYCTIGGSLSRSSQGTLVTVSSGGGGSGGGGSGGGGSEVQLVESG | |
| | GGLVQPGGSLRLSCAASGRAFSDYAMAWFRQAPGQEREFVAGIGWS | |
| | GGDTLYADSVRGRFTNSRDNSKNTLYLQMNSLRAEDTAVYYCAARQ | |
| | GQYIYSSMRSDSYDYWGQGTLVTVSS | |
| CRL0492 | EVQLVESGGGLVKPGGSLRLSCAASGRPVSNYAAAWFRQAPGKERE | 85 |
| (C5/HSA) | FVSAINWQKTATYADSVKGRFTISRDNAKNSLYLQMNSLRAEDTAV | |
| | YYCAAVFRVVAPKTQYDYDYWGQGTLVTVSSGGGGSGGGGSGGGGS | |
| | EVQLVESGGGLVQPGGSLRLSCAASGRAFSDYAMAWFRQAPGQERE | |
| | FVAGIGWSGGDTLYADSVRGRFTNSRDNSKNTLYLQMNSLRAEDTA | |
| | VYYCAARQGQYIYSSMRSDSYDYWGQGTLVTVSS | |
| CRL0493 | EVQLLESGGGLVQPGGSLRLSCAASGFTFRSFGMSWVRQAPGKGPE | 86 |
| (C5/HSA) | WVSSISGSGSDTLYADSVKGRFTISRDNSKNTLYLQMNSLRPEDTA | |
| | VYYCTIGGSLSRSSQGTLVTVSSGGGGSGGGGSGGGGSEVQLVESG | |
| | GGLVQPGGSLRLSCAASGRAFSDYAMAWFRQAPGQEREFVAGIGWS | |
| | GGDTLYADSVRGRFTISRDNAKNTLYLQMNSLRAEDTAVYYCAARQ | |
| | GQYIYSSMRSDSYDYWGQGTLVTVSS | |
| CRL0494 | EVQLVESGGGLVKPGGSLRLSCAASGRPVSNYAAAWFRQAPGKERE | 87 |
| (C5/HSA) | FVSAINWQKTATYADSVKGRFTISRDNAKNSLYLQMNSLRAEDTAV | |
| | YYCAAVFRVVAPKTQYDYDYWGQGTLVTVSSGGGGSGGGGSGGGGS | |
| | EVQLVESGGGLVQPGGSLRLSCAASGRAFSDYAMAWFRQAPGQERE | |
| | FVAGIGWSGGDTLYADSVRGRFTISRDNAKNTLYLQMNSLRAEDTA | |
| | VYYCAARQGQYIYSSMRSDSYDYWGQGTLVTVSS | |
| CRL0495 | EVQLLESGGGLVQPGGSLRLSCAASGFTFRSFGMSWVRQAPGKGPE | 88 |
| (C5/HSA) | WVSSISGSGSDTLYADSVKGRFTISRDNSKNTLYLQMNSLRPEDTA | |
| | VYYCTIGGSLSRSSQGTLVTVSSGGGGSGGGGSGGGGSEVQLVESG | |
| | GGLVQPGGSLRLSCAASGRAFSDYAMAWFRQAPGQEREFVAGIGWS | |
| | GGDTLYADSVRGRFTISRDNSKNTMYLQMNSLRAEDTAVYYCAARQ | |
| | GQYIYSSMRSDSYDYWGQGTLVTVSS | |
| CRL0496 | EVQLVESGGGLVKPGGSLRLSCAASGRPVSNYAAAWFRQAPGKERE | 89 |
| (C5/HSA) | FVSAINWQKTATYADSVKGRFTISRDNAKNSLYLQMNSLRAEDTAV | |
| | YYCAAVFRVVAPKTQYDYDYWGQGTLVTVSSGGGGSGGGGSGGGGS | |
| | EVQLVESGGGLVQPGGSLRLSCAASGRAFSDYAMAWFRQAPGQERE | |
| | FVAGIGWSGGDTLYADSVRGRFTISRDNSKNTMYLQMNSLRAEDTA | |
| | VYYCAARQGQYIYSSMRSDSYDYWGQGTLVTVSS | |
| CRL0497 | EVQLLESGGGLVQPGGSLRLSCAASGFTFRSFGMSWVRQAPGKGPE | 90 |
| (HSA/C5) | WVSSISGSGSDTLYADSVKGRFTISRDNSKNTLYLQMNSLRPEDTA | |
| | VYYCTIGGSLSRSSQGTLVTVSSGGGGSGGGGSGGGGSEVQLVESG | |
| | GGLVQPGGSLRLSCAASGRAFSDYAMAWFRQAPGQGLEFVAGIGWS | |
| | GGDTLYADSVRGRFTISRDNSKNTLYLQMNSLRAEDTAVYYCAARQ | |
| | GQYIYSSMRSDSYDYWGQGTLVTVSS | |
| CRL0498 | EVQLVESGGGLVKPGGSLRLSCAASGRPVSNYAAAWFRQAPGKERE | 91 |
| (HSA/C5) | FVSAINWQKTATYADSVKGRFTISRDNAKNSLYLQMNSLRAEDTAV | |
| | YYCAAVFRVVAPKTQYDYDYWGQGTLVTVSSGGGGSGGGGSGGGGS | |
| | EVQLVESGGGLVQPGGSLRLSCAASGRAFSDYAMAWFRQAPGQGLE | |
| | FVAGIGWSGGDTLYADSVRGRFTISRDNSKNTLYLQMNSLRAEDTA | |
| | VYYCAARQGQYIYSSMRSDSYDYWGQGTLVTVSS | |
| CRL0499 | EVQLLESGGGLVQPGGSLRLSCAASGFTFRSFGMSWVRQAPGKGPE | 92 |
| (HSA/C5) | WVSSISGSGSDTLYADSVKGRFTISRDNSKNTLYLQMNSLRPEDTA | |
| | VYYCTIGGSLSRSSQGTLVTVSSGGGGSGGGGSGGGGSEVQLVESG | |
| | GGLVQPGGSLRLSCAASGRAHSDYAMAWFRQAPGQEREFVAGIGWS | |
| | GGDTLYADSVRGRFTNSRDNSKNTLYLQMNSLRAEDTAVYYCAARQ | |
| | GQYIYSSMRSDSYDYWGQGTLVTVSS | |
| CRL0500 | EVQLVESGGGLVKPGGSLRLSCAASGRPVSNYAAAWFRQAPGKERE | 93 |
| (HSA/C5) | FVSAINWQKTATYADSVKGRFTISRDNAKNSLYLQMNSLRAEDTAV | |
| | YYCAAVFRVVAPKTQYDYDYWGQGTLVTVSSGGGGSGGGGSGGGGS | |
| | EVQLVESGGGLVQPGGSLRLSCAASGRAHSDYAMAWFRQAPGQERE | |
| | FVAGIGWSGGDTLYADSVRGRFTNSRDNSKNTLYLQMNSLRAEDTA | |
| | VYYCAARQGQYIYSSMRSDSYDYWGQGTLVTVSS | |
| CRL0501 | EVQLLESGGGLVQPGGSLRLSCAASGFTFRSFGMSWVRQAPGKGPE | 94 |
| (HSA/C5) | WVSSISGSGSDTLYADSVKGRFTISRDNSKNTLYLQMNSLRPEDTA | |
| | VYYCTIGGSLSRSSQGTLVTVSSGGGGSGGGGSGGGGSEVQLVESG | |
| | GGLVQPGGSLRLSCAASGRAHSDYAMAWFRQAPGQGLEFVAGIGWS | |
| | GGDTLYADSVRGRFTISRDNSKNTLYLQMNSLRAEDTAVYYCAARQ | |
| | GQYIYSSMRSDSYDYWGQGTLVTVSS | |
| CRL0502 | EVQLVESGGGLVKPGGSLRLSCAASGRPVSNYAAAWFRQAPGKERE | 95 |
| (HSA/C5) | FVSAINWQKTATYADSVKGRFTISRDNAKNSLYLQMNSLRAEDTAV | |
| | YYCAAVFRVVAPKTQYDYDYWGQGTLVTVSSGGGGSGGGGSGGGGS | |
| | EVQLVESGGGLVQPGGSLRLSCAASGRAHSDYAMAWFRQAPGQGLE | |
| | FVAGIGWSGGDTLYADSVRGRFTISRDNSKNTLYLQMNSLRAEDTA | |
| | VYYCAARQGQYIYSSMRSDSYDYWGQGTLVTVSS | |

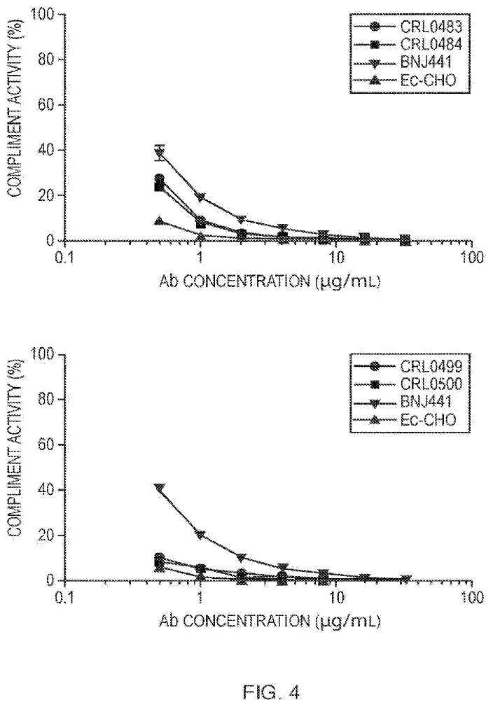

Four bispecific molecules CRL0483, CRL0484, CRL0499, and CRL0500 were prioritized based on binding and functional assays. Biacore affinity measurements for binding to human C5 for CRL0483, CRL0484, CRL0499, and CRL0500 are shown in Table 10 and functional assessments are shown in in FIGS. 3, 4 and 5. These four bispecific molecules were evaluated in in vivo pharmacokinetic studies in cynomolgus monkeys.

TABLE 10
Biacore measurements of prioritized fusions at pH 7.4 and pH 6.0

| | | | kₐ | k_{d} | K_{D} | |
|---|---|---|---|---|---|---|
| Sample | C5 | pH | (1/Ms) | (1/s) | (M) | Chi² |
| CRL0483 | hC5 | 7.4 | 2.25e5 | 2.42e−4 | 1.07e−9 | 0.03 |
| | cC5 | 7.4 | 9.15e4 | 2.20e−5 | 2.40e−10 | 0.01 |
| CRL0484 | hC5 | 7.4 | 7.01e4 | 7.69e−5 | 1.10e−9 | 0.04 |
| | cC5 | 7.4 | 9.15e4 | 2.2e−5 | 2.40e−10 | 0.01 |
| CRL0499 | hC5* | 7.4 | 2.22e6 | 3.32e−4 | 1.5e−10 | 3.3 |
| | cC5 | 7.4 | N.D. | N.D. | N.D. | N.D. |
| CRL0500 | hC5 | 7.4 | 2.88e6 | 6.72e−4 | 2.33e−10 | 0.65 |
| | cC5 | 7.4 | 2.00e6 | 8.48e−4 | 4.2e−10 | 0.04 |
| CRL0483 | hC5 | 6.0 | 4.00e4 | 2.11e−04 | 5.27e−09 | 0.02 |
| | cC5 | 6.0 | 3.71e4 | 4.62e−5 | 1.25e−9 | 0.02 |
| CRL0484 | hC5 | 6.0 | 4.25e5 | 2.36e−4 | 5.56e−10 | 0.02 |
| | cC5 | 6.0 | 4.82e4 | 6.17e−6 | 1.28e−10 | 0.03 |
| CRL0499 | hC5* | 6.0 | 2.51e6 | 1.12e−3 | 4.48e−10 | 0.24 |
| | cC5 | 6.0 | 1.92e6 | 3.88e−3 | 2.02e−9 | 0.31 |
| CRL0500 | hC5* | 6.0 | 8.02e6 | 1.519e−3 | 1.89e−10 | 1.06 |
| | cC5* | 6.0 | 3.91e6 | 2.5e−3 | 6.41e−10 | 3.16 |

### Example 13. Pharmacokinetic Analysis of Bispecific Fusion Proteins

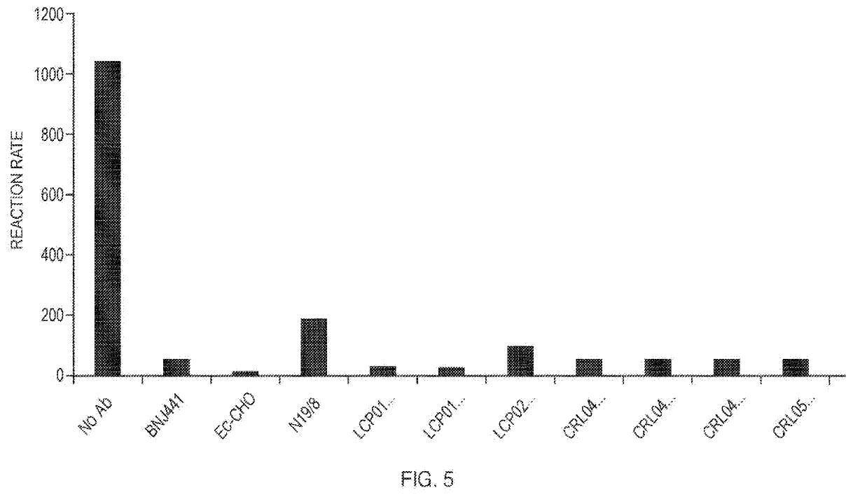

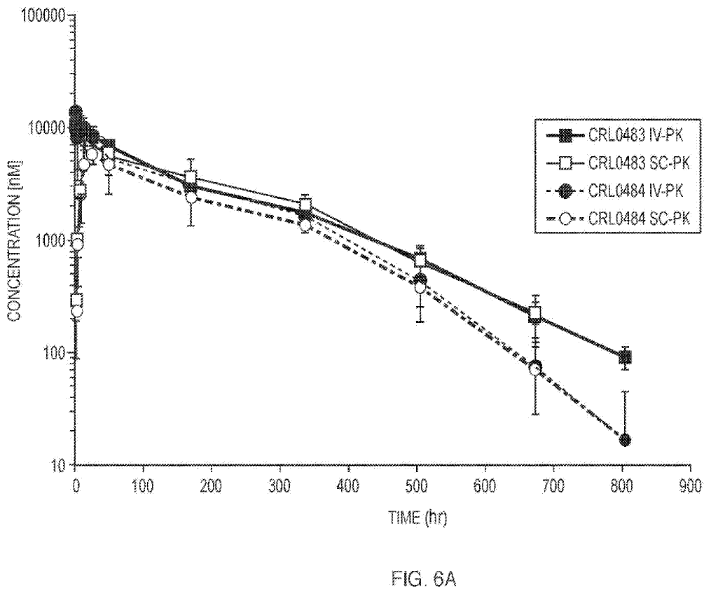

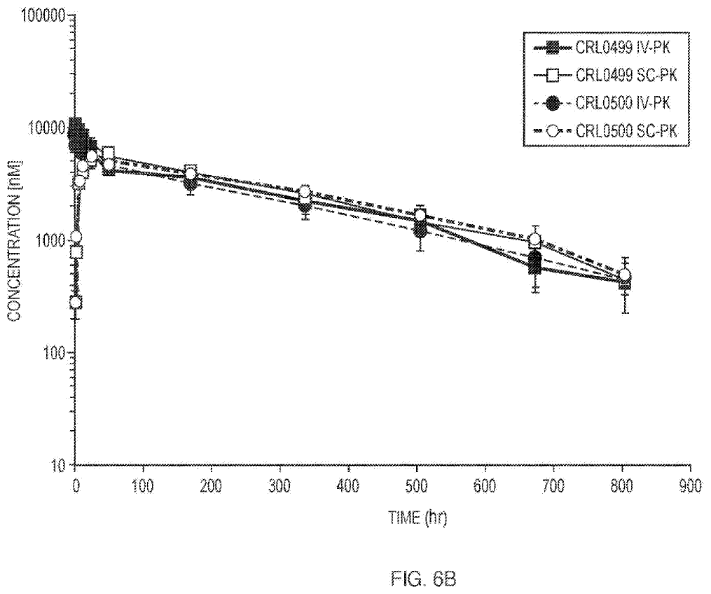

Purified proteins were dosed at 10 mg/kg either intravenously or subcutaneously in cynomolgus monkeys. Three monkeys per dose group per test article were used. Pharmacokinetics properties of bispecific molecules were measured by LC-MS based quantitation using signature peptides to each construct. The PK profile is shown in FIG. 6, and the parameters are described in Table 11.

TABLE 11
PK parameters after 10 mg/kg of test articles in cynomolgus monkeys

| | | | | AUC | C_{L} | | |
|---|---|---|---|---|---|---|---|
| | t_{1/2} | tₘₐₓ | Cₘₐₓ | (h * | (mL/ | V | F |
| Test article | (h) | (h) | (μg/mL) | μg/mL) | h/kg) | (mL/kg) | (%) |
| CRL0483 IV | 139 | 1.33 | 324 | 47900 | 0.211 | 42.0 | |
| CRL0484 IV | 125 | 1 | 382 | 43700 | 0.238 | 43.0 | |
| CRL0483 SC | 103 | 20 | 238 | 46412 | 0.218 | 32.5 | 97 |
| CRL0484 SC | 75.9 | 24 | 161 | 32610 | 0.315 | 34.9 | 75 |
| CRL0499 IV | 170 | 2.11 | 299 | 53773 | 0.184 | 46.9 | |
| CRL0500 IV | 239 | 0.167 | 351 | 51929 | 0.205 | 62.5 | |
| CRL0499 SC | 220 | 32 | 146 | 58666 | 0.173 | 54.2 | 109 |
| CRL0500 SC | 209 | 32 | 161 | 61475 | 0.163 | 49.0 | 118 |

Variant linker sequences were also generated for the bispecific fusion proteins. The sequences including these variant linker sequences are shown in Table 12.

TABLE 12
Sequences of anti-C5/anti-albumin bi-specifics with different linkers

| Name | Sequence | SEQ ID NO |
|---|---|---|
| CRL0952 | EVQLVESGGGLVKPGGSLRLSCAASGRPVSNYAAAWFRQAPGKEREFVS | 96 |
| | AINWQKTATYADSVKGRFTISRDNAKNSLYLQMNSLRAEDTAVYYCAAV | |
| | FRVVAPKTQYDYDYWGQGTLVTVSSGGGGAGGGGAGGGGSEVQLVESGG | |
| | GLVQPGGSLRLSCAASGRAHSDYAMAWFRQAPGQEREFVAGIGWSGGDT | |
| | LYADSVRGRFTNSRDNSKNTLYLQMNSLRAEDTAVYYCAARQGQYIYSS | |
| | MRSDSYDYWGQGTLVTVSS | |
| CRL0953 | EVQLVESGGGLVQPGGSLRLSCAASGRTFSGILSPYAVGWFRQAPGKGL | 97 |
| | EFVSTITSGGSAIYTDSVKGRFTISRDNAKDSLYLQMNSLRAEDTAVYY | |
| | CAVRTRRYGSNLGEVPQENEYGYWGQGTLVTVSSGGGGAGGGGAGGGGS | |
| | EVQLVESGGGLVKPGGSLRLSCAASGRPVSNYAAAWFRQAPGKEREFVS | |
| | AINWQKTATYADSVKGRFTISRDNAKNSLYLQMNSLRAEDTAVYYCAAV | |
| | FRVVAPKTQYDYDYWGQGTLVTVSS | |
| CRL0954 | EVQLVESGGGVVQAGDSLTLTCTAPVGTISDYGMGWFRQAPGKEREFVA | 98 |
| | SISWGGMWTDYADSVKGRFTISRDNDKNAVYLRMNSLNAEDTAVYYCGR | |
| | GRMYRGIGNSLAQPKSYGYWGQGTQVTVSSGGGGAGGGGAGGGGSEVQL | |
| | VESGGGLVKPGGSLRLSCAASGRPVSNYAAAWFRQAPGKEREFVSAINW | |
| | QKTATYADSVKGRFTISRDNAKNSLYLQMNSLRAEDTAVYYCAAVFRVV | |
| | APKTQYDYDYWGQGTLVTVSS | |
| CRL0955 | EVQLVESGGGLVQAGGSLRLSCAASGRTFSGILSAYAVGWFRQAPGKER | 99 |
| | EFVSTITSGGSTLSADSVKGRFTLSRDNAKDTVYLQMNSLKPEDTAVYY | |
| | CAVRTWPYGSNRGEVPTENEYGHWGQGTQVTVSSGGGGAGGGGAGGGGS | |
| | EVQLVESGGGLVKPGGSLRLSCAASGRPVSNYAAAWFRQAPGKEREFVS | |
| | AINWQKTATYADSVKGRFTISRDNAKNSLYLQMNSLRAEDTAVYYCAAV | |
| | FRVVAPKTQYDYDYWGQGTLVTVSS | |
| CRL0956 | EVQLVESGGGLVKPGGSLRLSCAASGRPVSNYAAAWFRQAPGKEREFVS | 100 |
| | AINWQKTATYADSVKGRFTISRDNAKNSLYLQMNSLRAEDTAVYYCAAV | |
| | FRVVAPKTQYDYDYWGQGTLVTVSSGGGGAGGGGAGGGGSEVQLVESGG | |
| | GVVQAGDSLTLTCTAPVGTISDYGMGWFRQAPGKEREFVASISWGGMWT | |
| | DYADSVKGRFTISRDNDKNAVYLRMNSLNAEDTAVYYCGRGRMYRGIGN | |
| | SLAQPKSYGYWGQGTQVTVSS | |
| CRL0957 | EVQLVESGGGLVKPGGSLRLSCAASGRPVSNYAAAWFRQAPGKEREFVS | 101 |
| | AINWQKTATYADSVKGRFTISRDNAKNSLYLQMNSLRAEDTAVYYCAAV | |
| | FRVVAPKTQYDYDYWGQGTLVTVSSGGGGAGGGGAGGGGSEVQLVESGG | |
| | GLVQAGGSLRLSCAASGRTFSGILSAYAVGWFRQAPGKEREFVSTITSG | |
| | GSTLSADSVKGRFTLSRDNAKDTVYLQMNSLKPEDTAVYYCAVRTWPYG | |
| | SNRGEVPTENEYGHWGQGTQVTVSS | |

### Example 14. Varying Peptide Linker Sequences

Constructs were generates using the HAS042 (SEQ ID NO:26) albumin binding domain and the CRL0305 (SEQ ID NO:11) humanized anti-C5 VHH. The constructs that were evaluated are listed in Table 13.

TABLE 13
Linkers used for generating fusion proteins.

| | | SEQ | Octet Binding- |
|---|---|---|---|
| | | ID | Human C5 and |
| Protein | Linker | NO | Human Albumin |
| TPP-3211 | No anti-albumin | | no |
| | domain (only anti-C5) | | |
| TPP-3212 | No anti-C5 domain | | no |
| | (only anti-albumin) | | |
| TPP-3213 | No linker | | yes |
| TPP-3214 | GGGGS | 104 | yes |
| TPP-3215 | EAAAKEAAAKEAAAK | 110 | yes |
| TPP-3216 | PAPAP | 111 | yes |
| TPP-3217 | GGGGSPAPAP | 112 | yes |
| TPP-3218 | PAPAPGGGGS | 113 | yes |
| TPP-3219 | GSTSGKSSEGKG | 114 | yes |
| TPP-3220 | GGGDSGGGDS | 115 | yes |
| TPP-3221 | GGGESGGGES | 116 | yes |
| TPP-3222 | GGGGSGGGGS | 105 | yes |
| TPP-3223 | GGGDSGGGGS | 117 | yes |
| TPP-3224 | GGGASGGGGS | 118 | yes |
| TPP-3225 | GGGESGGGGS | 119 | yes |
| TPP-3226 | ASTKGP | 120 | yes |
| TPP-3227 | ASTKGPSVFPLAP | 121 | yes |
| TPP-3228 | GGGGGGGP | 123 | yes |
| TPP-3229 | GGGGGGGGP | 321 | yes |
| TPP-3230 | PAPNLLGGP | 124 | yes |
| TPP-3231 | PNLLGGP | 323 | yes |
| TPP-3232 | GGGGGG | 125 | yes |
| TPP-3233 | GGGGGGGGGGGG | 126 | yes |
| TPP-3234 | APELPGGP | 127 | yes |
| TPP-3235 | SEPQPQPG | 128 | yes |
| TPP-1252 | GGGGSGGGGSGGGGS | 106 | yes |

The 26 constructs listed in Table 13 were expressed and the fusion proteins were evaluated for binding to human C5 and albumin (Table 13-Octet binding), generation of aggregates, hydrophobicity (HIC HPLC) and glycosylation (electrospray mass spectrometry). For the octet analysis, biotinylated human C5 was captured on a CAP chip followed by an injection of a test bi-specific molecule. Various concentrations of albumin were subsequently injected. Kinetics were determined at pH 7.4 (Biacore 3000). All bi-specific molecules bound to both C5 and albumin, with each having a similar affinity for albumin (5-6 nM).

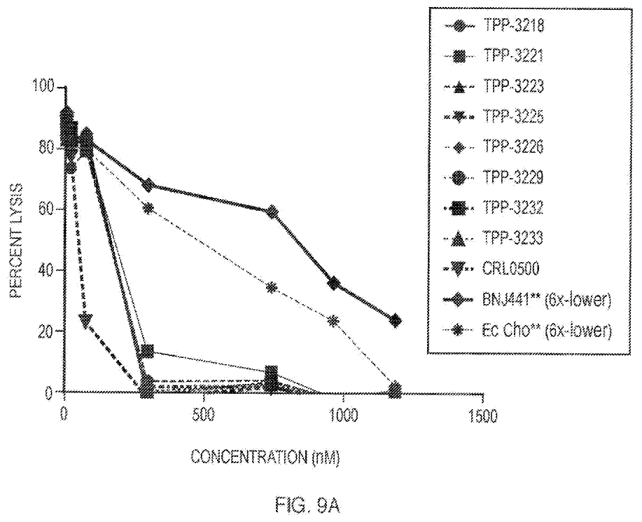

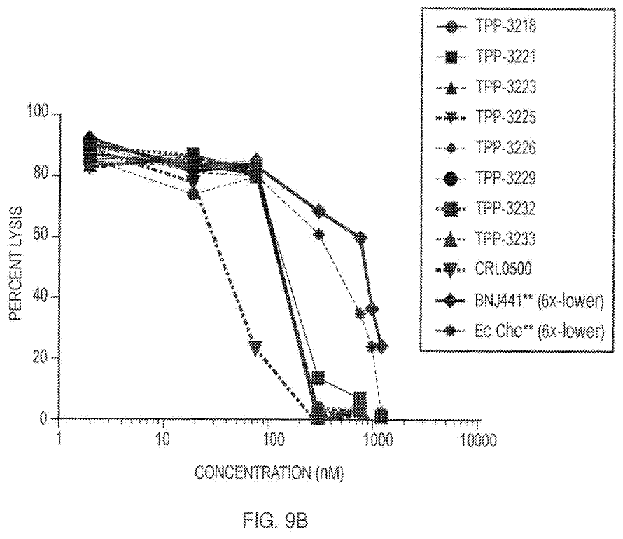

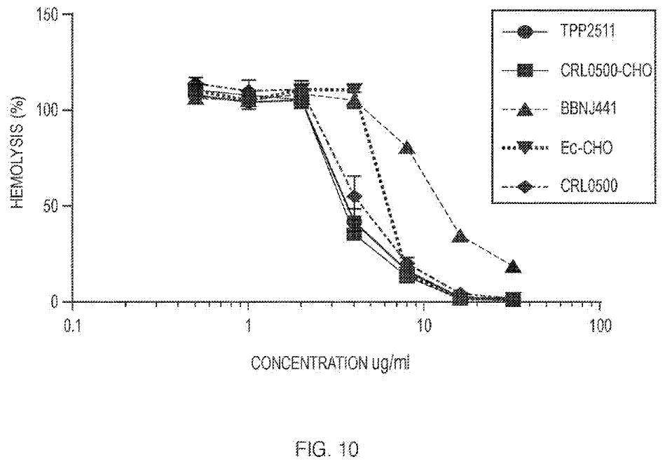

The bi-specific fusion proteins were tested for their ability to inhibit hemolysis in an in vitro hemolysis assay. Data are shown in FIGS. 9A and 9B.

Table 14 shows binding kinetics for CRL0500 and CRL0952 binding to human C5 (hC5) and cynomolgus C5 (cC5).

TABLE 14
Kinetics of bi-specific binding to C5

| Sample | Antigen | pH | kₐ (1/Ms) | k_{d} (1/s) | K_{D} (M) | Chi² |
|---|---|---|---|---|---|---|
| CRL0500 | hC5 | 7.4 | 9.60e+06 | 4.91e−04 | 5.12e−11 | 0.24 |
| CRL0500 | cC5 | 7.4 | 3.74e+06 | 8.18e−04 | 2.19e−10 | 0.01 |
| CRL0952 | hC5 | 7.4 | 1.01e+07 | 5.39e−04 | 5.36e−11 | 0.27 |
| CRL0952 | cC5 | 7.4 | 3.53e+06 | 7.86e−04 | 2.23e−10 | 0.01 |
| CRL0500 | hC5 | 6.0 | 7.56e+06 | 1.04e−03 | 1.38e−10 | 0.54 |
| CRL0500 | cC5 | 6.0 | 5.51e+06 | 4.10e−03 | 7.44e−10 | 0.07 |
| CRL0952 | hC5 | 6.0 | 5.84e+06 | 9.07e−04 | 1.55e−10 | 0.58 |
| CRL0952 | cC5 | 6.0 | 5.55e+06 | 3.99e−03 | 7.20e−10 | 0.06 |

Table 15 shows binding kinetics for CRL0500 and CRL0952 binding to Plasbumin® and cynomolgus albumin.

TABLE 15
Albumin bi-specific kinetics

| Sample | Albumin | pH | kₐ (1/Ms) | k_{d} (1/s) | K_{D} (M) | Chi² |
|---|---|---|---|---|---|---|
| CRL0500 | Plasbumin | 7.4 | 3.70e06 | 3.46e−03 | 9.36e−10 | 0.30 |
| CRL0500 | Plasbumin | 6.0 | 3.55e06 | 2.0e−03 | 5.63e−10 | 0.17 |
| CRL0952 | Plasbumin | 7.4 | 3.98e06 | 3.59e−03 | 9.01e−10 | 0.21 |
| CRL0952 | Plasbumin | 6.0 | 3.23e06 | 2.10e−03 | 6.49e−10 | 0.10 |
| CRL0500 | cyno | 7.4 | 3.32e06 | 1.26e−02 | 3.78e−09 | 0.42 |
| CRL0500 | cyno | 6.0 | 3.27e06 | 6.93e−03 | 2.12e−09 | 0.43 |
| CRL0952 | cyno | 7.4 | 2.93e06 | 1.52e−02 | 5.19e−09 | 0.17 |
| CRL0952 | cyno | 6.0 | 3.03e06 | 7.55e−03 | 2.49e−09 | 0.22 |

### Example 15. pH-Dependent Binding of Anti-C5 VHH Domains

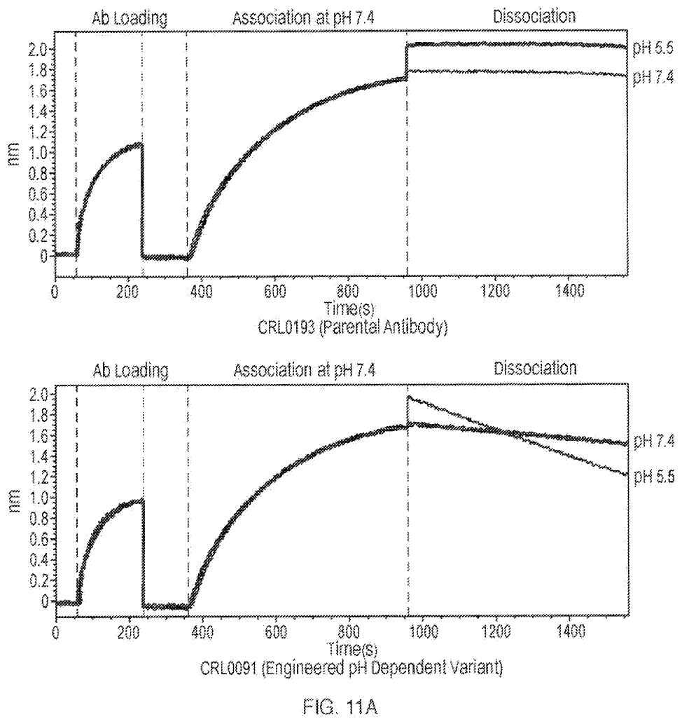

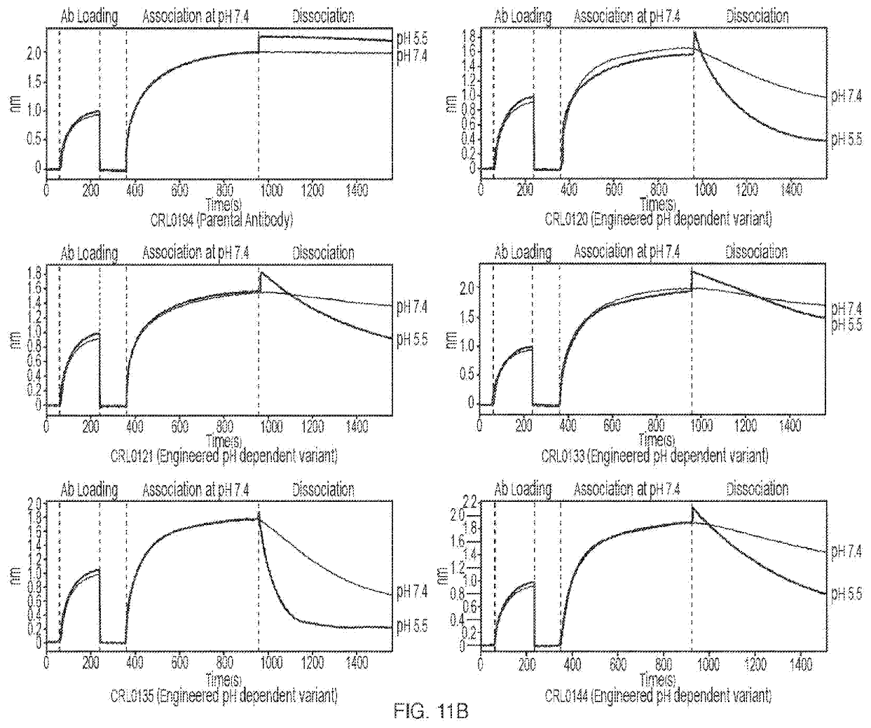

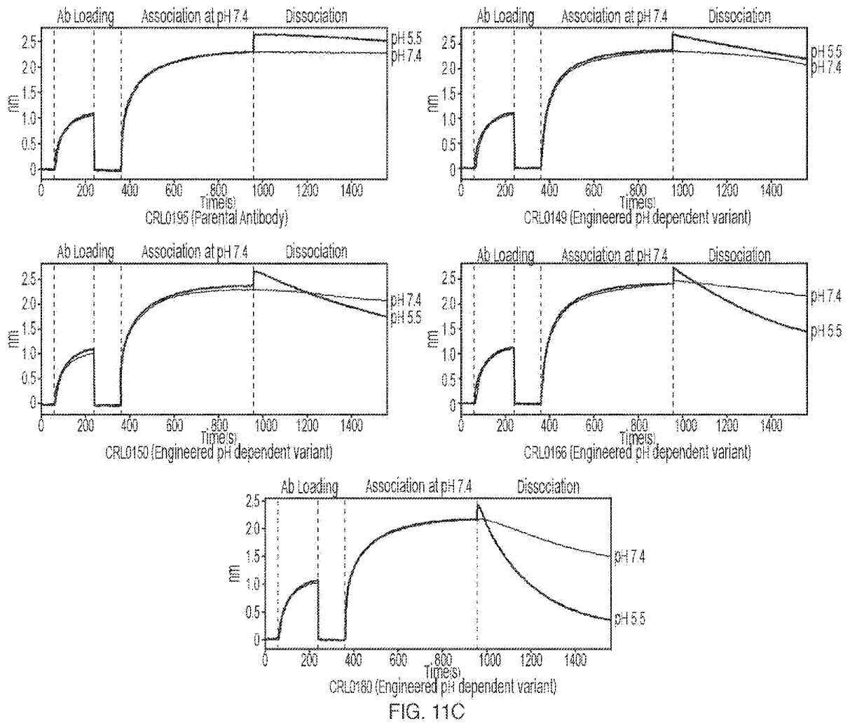

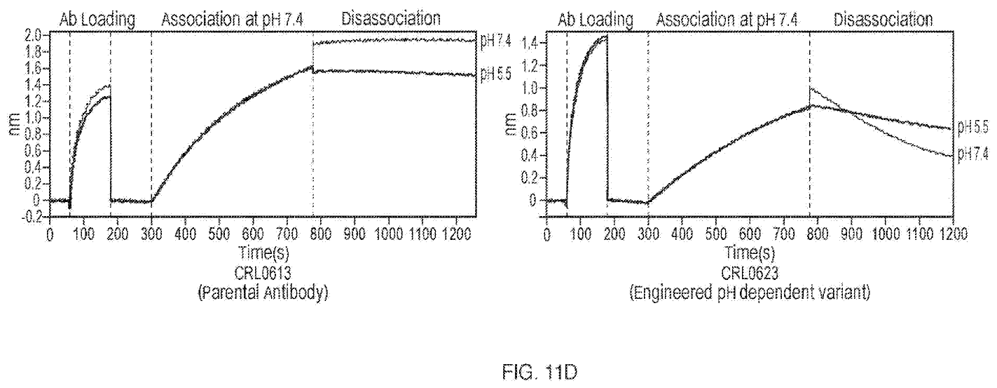

Histidine scanning was performed across all CDRs for anti-C5 VHH domains LCP0115, LCP0143, LCP0146 and LCP0302. Single histidine substitutions were generated at each position in the CDRs (shown in bold, underlined text). Variants were transfected in Expi293 cell culture and evaluated for pH-dependent binding at pH 7.4, 6.0 and 5.5. Several variants from each antibody exhibited pH-dependent binding. These variants are listed in Table 16 and their pH-dependent binding response is illustrated in FIGS. 11A-D.

TABLE 16
Pre-humanized histidine scanned variants of anti-C5 VHH domains.

| Variant name | Histidine variant sequence | SEQ ID NO |
|---|---|---|
| LCP0115 | | |
| variants | | |
| CRL0085 | EVQLVESGGGLVQAGGSLRLSCAASGRTFSGILSPYAVGWFRQ | 281 |
| | APGKGREFVSTITSGGSAIYTDSVKGRFTLSRDNAKDTVYLQM | |
| | NSLKPEDTAVYYCHVRTRRYGSNLGEVPQENEYGYWGQGTQVT | |
| | VSS | |
| CRL0091 | EVQLVESGGGLVQAGGSLRLSCAASGRTFSGILSPYAVGWFRQ | 282 |
| | APGKGREFVSTITSGGSAIYTDSVKGRFTLSRDNAKDTVYLQM | |
| | NSLKPEDTAVYYCAVRTRRHGSNLGEVPQENEYGYWGQGTQVT | |
| | VSS | |
| LCP0143 | | |
| variants | | |
| CRL0120 | EVQLVESGGGLVQAGGSLRLSCAAPEMGATINVMAWYRQAPGK | 283 |
| | QRELVARLPHDNNIDYGDFAKGRFTISRDITRNTVYLQMNNLK | |
| | PDDTAVYYCNVLLSRQINGAYVHWGQGTQVTVSS | |
| CRL0121 | EVQLVESGGGLVQAGGSLRLSCAAPEMGATINVMAWYRQAPGK | 284 |
| | QRELVARLPLHNNIDYGDFAKGRFTISRDITRNTVYLQMNNLK | |
| | PDDTAVYYCNVLLSRQINGAYVHWGQGTQVTVSS | |
| CRL0133 | EVQLVESGGGLVQAGGSLRLSCAAPEMGATINVMAWYRQAPGK | 285 |
| | QRELVARLPLDNNIDYGDFAKGRFTISRDITRNTVYLQMNNLK | |
| | PDDTAVYYCHVLLSRQINGAYVHWGQGTQVTVSS | |
| CRL0135 | EVQLVESGGGLVQAGGSLRLSCAAPEMGATINVMAWYRQAPGK | 286 |
| | QRELVARLPLDNNIDYGDFAKGRFTISRDITRNTVYLQMNNLK | |
| | PDDTAVYYCNVHLSRQINGAYVHWGQGTQVTVSS | |
| CRL0144 | EVQLVESGGGLVQAGGSLRLSCAAPEMGATINVMAWYRQAPGK | 287 |
| | QRELVARLPLDNNIDYGDFAKGRFTISRDITRNTVYLQMNNLK | |
| | PDDTAVYYCNVLLSRQINGAHVHWGQGTQVTVSS | |
| LCP0146 | | |
| variants | | |
| CRL0149 | EVQLVESGGGLVQAGGSLRLSCAASGRHFSDYAMAWFRQAPGK | 288 |
| | EREFVAGIGWSGGDTLYADSVRGRFTNSKDNAKNRMSLQMNSL | |
| | KPEDTAVYYCAARQGQYIYSSMRSDSYDYWGQGTQVTVSS | |
| CRL0150 | EVQLVESGGGLVQAGGSLRLSCAASGRAHSDYAMAWFRQAPGK | 289 |
| | EREFVAGIGWSGGDTLYADSVRGRFTNSKDNAKNRMSLQMNSL | |
| | KPEDTAVYYCAARQGQYIYSSMRSDSYDYWGQGTQVTVSS | |
| CRL0166 | EVQLVESGGGLVQAGGSLRLSCAASGRAFSDYAMAWFRQAPGK | 290 |
| | EREFVAGIGWSGGDTHYADSVRGRFTNSKDNAKNRMSLQMNSL | |
| | KPEDTAVYYCAARQGQYIYSSMRSDSYDYWGQGTQVTVSS | |
| CRL0180 | EVQLVESGGGLVQAGGSLRLSCAASGRAFSDYAMAWFRQAPGK | 291 |
| | EREFVAGIGWSGGDTLYADSVRGRFTNSKDNAKNRMSLQMNSL | |
| | KPEDTAVYYCAARQGQHIYSSMRSDSYDYWGQGTQVTVSS | |
| LCP0302 | | |
| variants | | |
| CRL0623 | EVQLVESGGGLVQAGGSLRLSCAASGRTFSGILSHYAVGWFRQ | 292 |
| | APGKEREFVSTITSGGSTLSADSVKGRFTLSRDNAKDTVYLQM | |
| | NSLKPEDTAVYYCAVRTWPYGSNRGEVPTENEYGHWGQGTQVT | |
| | VSS | |

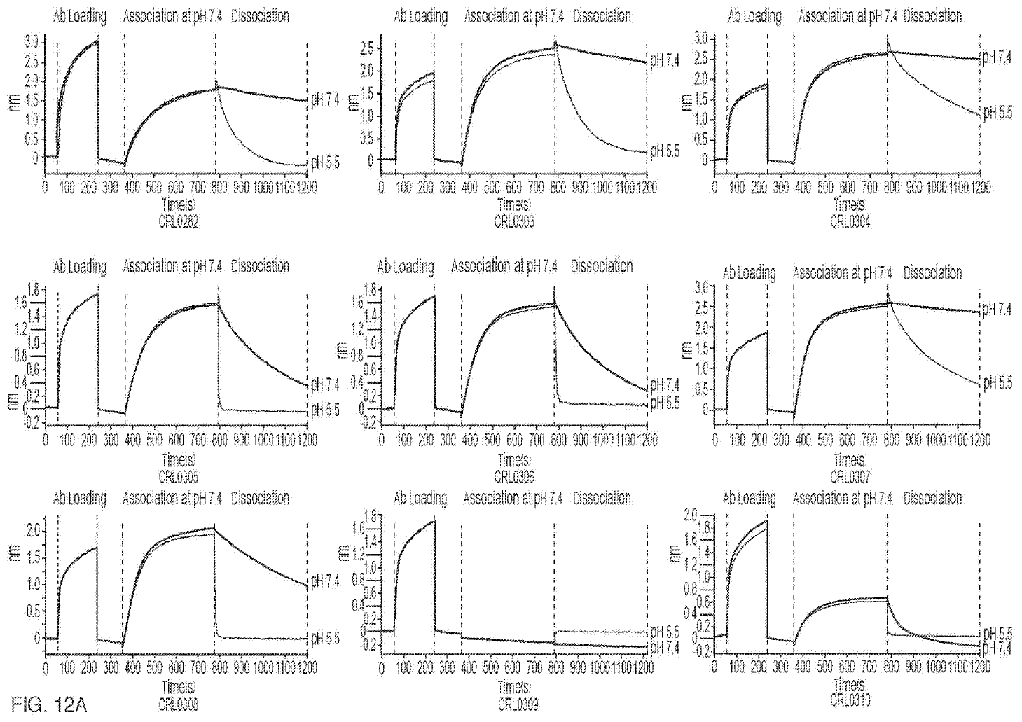

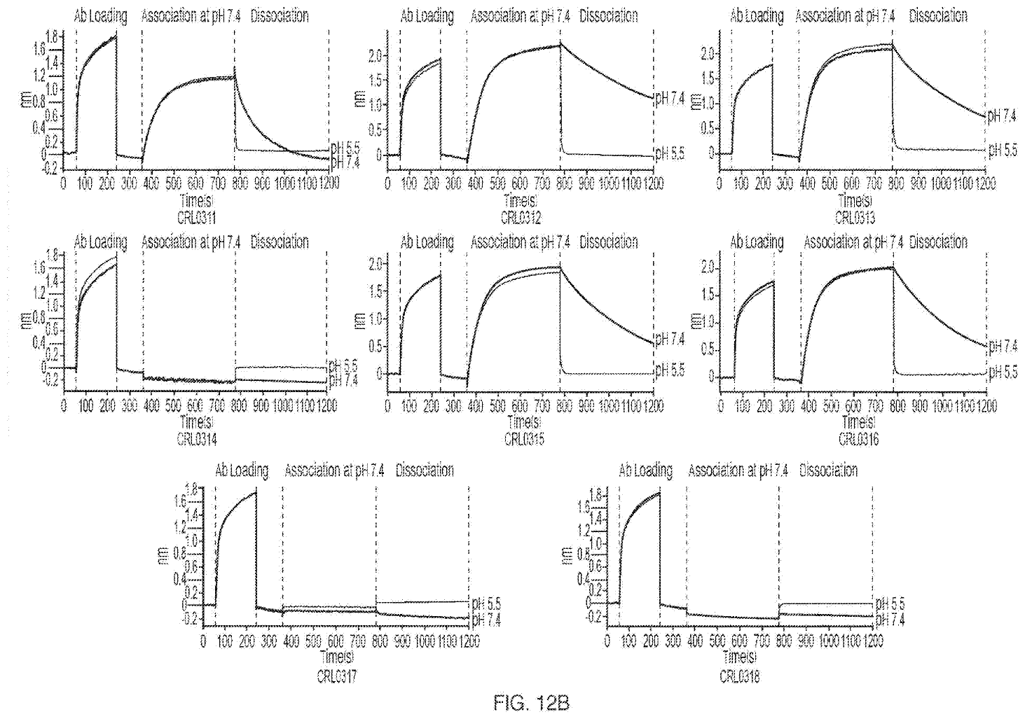

Single histidine mutations identified for pH-dependent binding were combined to enhance pH sensitivity. The sequences of these variants are shown in Table 17. These variants were evaluated in biolayer interferometry for pH-dependent binding and results are shown in FIGS. 12A and 12B.

TABLE 17
Histidine scanning combination variants of humanized anti-C5 VHH domains

| Variant name | Histidine variant sequence | SEQ ID NO |
|---|---|---|
| LCP0115 | | |
| combination | | |
| variants | | |
| CRL0282 | EVQLVESGGGLVQPGGSLRLSCAASGRTFSGILSPYAVGWFRQAPGKG | 293 |
| | LEFVSTITSGGSAIYTDSVKGRFTISRDNAKNSLYLQMNSLRAEDTAV | |
| | YYCAVRTRRHGSNLGEVPQENEYGYWGQGTLVTVSS | |
| LCP0146 | | |
| combination | | |
| variants | | |
| CRL0303 | EVQLVESGGGLVQPGGSLRLSCAASGRHFSDYAMAWFRQAPGQEREFV | 9 |
| | AGIGWSGGDTLYADSVRGRFTISRDNSKNTLYLQMNSLRAEDTAVYYC | |
| | AARQGQYIYSSMRSDSYDYWGQGTLVTVSS | |
| CRL0304 | EVQLVESGGGLVQPGGSLRLSCAASGRAHSDYAMAWFRQAPGQEREFV | 10 |
| | AGIGWSGGDTLYADSVRGRFTISRDNSKNTLYLQMNSLRAEDTAVYYC | |
| | AARQGQYIYSSMRSDSYDYWGQGTLVTVSS | |
| CRL0305 | EVQLVESGGGLVQPGGSLRLSCAASGRAFSDYAMAWFRQAPGQEREFV | 294 |
| | AGIGWSGGDTHYADSVRGRFTISRDNSKNTLYLQMNSLRAEDTAVYYC | |
| | AARQGQYIYSSMRSDSYDYWGQGTLVTVSS | |
| CRL0306 | EVQLVESGGGLVQPGGSLRLSCAASGRAFSDYAMAWFRQAPGQEREFV | 295 |
| | AGIGWSGGDTLYADSVRGRFTISRDNSKNTLYLQMNSLRAEDTAVYYC | |
| | AARQGQHIYSSMRSDSYDYWGQGTLVTVSS | |
| CRL0307 | EVQLVESGGGLVQPGGSLRLSCAASGRHHSDYAMAWFRQAPGQEREFV | 12 |
| | AGIGWSGGDTLYADSVRGRFTISRDNSKNTLYLQMNSLRAEDTAVYYC | |
| | AARQGQYIYSSMRSDSYDYWGQGTLVTVSS | |
| CRL0308 | EVQLVESGGGLVQPGGSLRLSCAASGRAHSDYAMAWFRQAPGQEREFV | 296 |
| | AGIGWSGGDTHYADSVRGRFTISRDNSKNTLYLQMNSLRAEDTAVYYC | |
| | AARQGQYIYSSMRSDSYDYWGQGTLVTVSS | |
| CRL0309 | EVQLVESGGGLVQPGGSLRLSCAASGRAFSDYAMAWFRQAPGQEREFV | 297 |
| | AGIGWSGGDTHYADSVRGRFTISRDNSKNTLYLQMNSLRAEDTAVYYC | |
| | AARQGQHIYSSMRSDSYDYWGQGTLVTVSS | |
| CRL0310 | EVQLVESGGGLVQPGGSLRLSCAASGRHFSDYAMAWFRQAPGQEREFV | 298 |
| | AGIGWSGGDTHYADSVRGRFTISRDNSKNTLYLQMNSLRAEDTAVYYC | |
| | AARQGQYIYSSMRSDSYDYWGQGTLVTVSS | |
| CRL0311 | EVQLVESGGGLVQPGGSLRLSCAASGRHFSDYAMAWFRQAPGQEREFV | 299 |
| | AGIGWSGGDTLYADSVRGRFTISRDNSKNTLYLQMNSLRAEDTAVYYC | |
| | AARQGQHIYSSMRSDSYDYWGQGTLVTVSS | |
| CRL0312 | EVQLVESGGGLVQPGGSLRLSCAASGRAHSDYAMAWFRQAPGQEREFV | 296 |
| | AGIGWSGGDTHYADSVRGRFTISRDNSKNTLYLQMNSLRAEDTAVYYC | |
| | AARQGQYIYSSMRSDSYDYWGQGTLVTVSS | |
| CRL0313 | EVQLVESGGGLVQPGGSLRLSCAASGRAHSDYAMAWFRQAPGQEREFV | 300 |
| | AGIGWSGGDTLYADSVRGRFTISRDNSKNTLYLQMNSLRAEDTAVYYC | |
| | AARQGQHIYSSMRSDSYDYWGQGTLVTVSS | |
| CRL0314 | EVQLVESGGGLVQPGGSLRLSCAASGRAFSDYAMAWFRQAPGQEREFV | 297 |
| | AGIGWSGGDTHYADSVRGRFTISRDNSKNTLYLQMNSLRAEDTAVYYC | |
| | AARQGQHIYSSMRSDSYDYWGQGTLVTVSS | |
| CRL0315 | EVQLVESGGGLVQPGGSLRLSCAASGRHHSDYAMAWFRQAPGQEREFV | 301 |
| | AGIGWSGGDTHYADSVRGRFTISRDNSKNTLYLQMNSLRAEDTAVYYC | |
| | AARQGQYIYSSMRSDSYDYWGQGTLVTVSS | |
| CRL0316 | EVQLVESGGGLVQPGGSLRLSCAASGRHHSDYAMAWFRQAPGQEREFV | 302 |
| | AGIGWSGGDTLYADSVRGRFTISRDNSKNTLYLQMNSLRAEDTAVYYC | |
| | AARQGQHIYSSMRSDSYDYWGQGTLVTVSS | |
| CRL0317 | EVQLVESGGGLVQPGGSLRLSCAASGRAHSDYAMAWFRQAPGQEREFV | 303 |
| | AGIGWSGGDTHYADSVRGRFTISRDNSKNTLYLQMNSLRAEDTAVYYC | |
| | AARQGQHIYSSMRSDSYDYWGQGTLVTVSS | |
| CRL0318 | EVQLVESGGGLVQPGGSLRLSCAASGRHFSDYAMAWFRQAPGQEREFV | 304 |
| | AGIGWSGGDTHYADSVRGRFTISRDNSKNTLYLQMNSLRAEDTAVYYC | |
| | AARQGQHIYSSMRSDSYDYWGQGTLVTVSS | |

### Example 16. Generation of Anti-C5 and Anti-Albumin Bispecific Fusions

Anti-C5 VHH domains were fused to an anti-albumin domain to generate bispecific molecules. Four different linker lengths (G₄S)₃ (SEQ ID NO: 106), (G₄S)₄ (SEQ ID NO: 107), (G₄S)₅ (SEQ ID NO: 108) and (G₄S)₆ (SEQ ID NO: 109) and two different orientations (N-terminal or C-terminal) of anti-albumin domain were evaluated. The sequences of the generated molecules are shown in Table 18. Constructs were expressed in HEK293F cells and purified using Protein A affinity chromatography. Purified fusion molecules were evaluated in Biacore experiments. Human C5 was biotinylated and immobilized on a biacore chip, purified bispecific molecules were injected to saturate the chip followed by three different concentrations of human serum albumin to obtain kinetics. Measured affinity to human serum albumin was used as a proxy to compare the different linker lengths. (G₄S)₃ (SEQ ID NO: 106) was chosen as the optimal linker length to generate bispecific fusions. N-or C-terminal anti-albumin fusion was also evaluated in the same experiment. Different orientations were found to be optimal for different anti-C5 VHH domains.

TABLE 8
Sequences of Linker length and Orientation Variants of
anti-C5/anti-albumin bi-specifics

| Name | Sequence | SEQ ID NO |
|---|---|---|
| CRL0248 | EVQLVESGGGLVQPGGSLRLSCAASGRTFSGILSPYAVGWFRQAPGKGLEFVST | 305 |
| | ITSGGSAIYTDSVKGRFTISRDNAKNSLYLQMNSLRAEDTAVYYCAVRTRRYGS | |
| | NLGEVPQENEYGYWGQGTLVTVSSGGGGSGGGGSGGGGSEVQLLESGGGLVQPG | |
| | GSLRLSCAASGFTFRSFGMSWVRQAPGKGPEWVSSISGSGSDTLYADSVKGRFT | |
| | ISRDNSKNTLYLQMNSLRPEDTAVYYCTIGGSLSRSSQGTLVTVSS | |
| CRL0249 | EVQLVESGGGLVQPGGSLRLSCAASGRTFSGILSPYAVGWFRQAPGKGLEFVST | 306 |
| | ITSGGSAIYTDSVKGRFTISRDNAKNSLYLQMNSLRAEDTAVYYCAVRTRRYGS | |
| | NLGEVPQENEYGYWGQGTLVTVSSGGGGSGGGGSGGGGSGGGGSEVQLLESGGG | |
| | LVQPGGSLRLSCAASGFTFRSFGMSWVRQAPGKGPEWVSSISGSGSDTLYADSV | |
| | KGRFTISRDNSKNTLYLQMNSLRPEDTAVYYCTIGGSLSRSSQGTLVTVSS | |
| CRL0250 | EVQLVESGGGLVQPGGSLRLSCAASGRTFSGILSPYAVGWFRQAPGKGLEFVST | 307 |
| | ITSGGSAIYTDSVKGRFTISRDNAKNSLYLQMNSLRAEDTAVYYCAVRTRRYGS | |
| | NLGEVPQENEYGYWGQGTLVTVSSGGGGSGGGGSGGGGSGGGGSGGGGSEVQLL | |
| | ESGGGLVQPGGSLRLSCAASGFTFRSFGMSWVRQAPGKGPEWVSSISGSGSDTL | |
| | YADSVKGRFTISRDNSKNTLYLQMNSLRPEDTAVYYCTIGGSLSRSSQGTLVTV | |
| | SS | |
| CRL0251 | EVQLVESGGGLVQPGGSLRLSCAASGRTFSGILSPYAVGWFRQAPGKGLEFVST | 308 |
| | ITSGGSAIYTDSVKGRFTISRDNAKNSLYLQMNSLRAEDTAVYYCAVRTRRYGS | |
| | NLGEVPQENEYGYWGQGTLVTVSSGGGGSGGGGSGGGGSGGGGSGGGGSGGGGS | |
| | EVQLLESGGGLVQPGGSLRLSCAASGFTFRSFGMSWVRQAPGKGPEWVSSISGS | |
| | GSDTLYADSVKGRFTISRDNSKNTLYLQMNSLRPEDTAVYYCTIGGSLSRSSQG | |
| | TLVTVSS | |
| CRL0254 | EVQLLESGGGLVQPGGSLRLSCAASGFTFRSFGMSWVRQAPGKGPEWVSSISGS | 309 |
| | GSDTLYADSVKGRFTISRDNSKNTLYLQMNSLRPEDTAVYYCTIGGSLSRSSQG | |
| | TLVTVSSGGGGSGGGGSGGGGSEVQLVESGGGLVQPGGSLRLSCAASGRTFSGI | |
| | LSPYAVGWFRQAPGKGLEFVSTITSGGSAIYTDSVKGRFTTSRDNAKNSLYLQM | |
| | NSLRAEDTAVYYCAVRTRRYGSNLGEVPQENEYGYWGQGTLVTVSS | |
| CRL0255 | EVQLLESGGGLVQPGGSLRLSCAASGFTFRSFGMSWVRQAPGKGPEWVSSISGS | 310 |
| | GSDTLYADSVKGRFTISRDNSKNTLYLQMNSLRPEDTAVYYCTIGGSLSRSSQG | |
| | TLVTVSSGGGGSGGGGSGGGGSGGGGSEVQLVESGGGLVQPGGSLRLSCAASGR | |
| | TFSGILSPYAVGWFRQAPGKGLEFVSTITSGGSAIYTDSVKGRFTISRDNAKNS | |
| | LYLQMNSLRAEDTAVYYCAVRTRRYGSNLGEVPQENEYGYWGQGTLVTVSS | |
| CRL0256 | EVQLLESGGGLVQPGGSLRLSCAASGFTFRSFGMSWVRQAPGKGPEWVSSISGS | 311 |
| | GSDTLYADSVKGRFTISRDNSKNTLYLQMNSLRPEDTAVYYCTIGGSLSRSSQG | |
| | TLVTVSSGGGGSGGGGSGGGGSGGGGSGGGGSEVQLVESGGGLVQPGGSLRLSC | |
| | AASGRTFSGILSPYAVGWFRQAPGKGLEFVSTITSGGSAIYTDSVKGRFTISRD | |
| | NAKNSLYLQMNSLRAEDTAVYYCAVRTRRYGSNLGEVPQENEYGYWGQGTLVTV | |
| | SS | |
| CRL0257 | EVQLLESGGGLVQPGGSLRLSCAASGFTFRSFGMSWVRQAPGKGPEWVSSISGS | 312 |
| | GSDTLYADSVKGRFTISRDNSKNTLYLQMNSLRPEDTAVYYCTIGGSLSRSSQG | |
| | TLVTVSSGGGGSGGGGSGGGGSGGGGSGGGGSGGGGSEVQLVESGGGLVQPGGS | |
| | LRLSCAASGRTFSGILSPYAVGWFRQAPGKGLEFVSTITSGGSAIYTDSVKGRF | |
| | TISRDNAKNSLYLQMNSLRAEDTAVYYCAVRTRRYGSNLGEVPQENEYGYWGQG | |
| | TLVTVSS | |
| CRL0272 | EVQLVESGGGLVQPGGSLRLSCAASGRAFSDYAMAWFRQAPGQEREFVAGIGWS | 313 |
| | GGDTLYADSVRGRFTISRDNSKNTLYLQMNSLRAEDTAVYYCAARQGQYIYSSM | |
| | RSDSYDYWGQGTLVTVSSGGGGSGGGGSGGGGSEVQLLESGGGLVQPGGSLRLS | |
| | CAASGFTFRSFGMSWVRQAPGKGPEWVSSISGSGSDTLYADSVKGRFTISRDNS | |
| | KNTLYLQMNSLRPEDTAVYYCTIGGSLSRSSQGTLVTVSS | |
| CRL0273 | EVQLVESGGGLVQPGGSLRLSCAASGRAFSDYAMAWFRQAPGQEREFVAGIGWS | 314 |
| | GGDTLYADSVRGRFTISRDNSKNTLYLQMNSLRAEDTAVYYCAARQGQYIYSSM | |
| | RSDSYDYWGQGTLVTVSSGGGGSGGGGSGGGGSGGGGSEVQLLESGGGLVQPGG | |
| | SLRLSCAASGFTFRSFGMSWVRQAPGKGPEWVSSISGSGSDTLYADSVKGRFTI | |
| | SRDNSKNTLYLQMNSLRPEDTAVYYCTIGGSLSRSSQGTLVTVSS | |
| CRL0274 | EVQLVESGGGLVQPGGSLRLSCAASGRAFSDYAMAWFRQAPGQEREFVAGIGWS | 315 |
| | GGDTLYADSVRGRFTISRDNSKNTLYLQMNSLRAEDTAVYYCAARQGQYIYSSM | |
| | RSDSYDYWGQGTLVTVSSGGGGSGGGGSGGGGSGGGGSGGGGSEVQLLESGGGL | |
| | VQPGGSLRLSCAASGFTFRSFGMSWVRQAPGKGPEWVSSISGSGSDTLYADSVK | |
| | GRFTISRDNSKNTLYLQMNSLRPEDTAVYYCTIGGSLSRSSQGTLVTVSS | |
| CRL0275 | EVQLVESGGGLVQPGGSLRLSCAASGRAFSDYAMAWFRQAPGQEREFVAGIGWS | 316 |
| | GGDTLYADSVRGRFTISRDNSKNTLYLQMNSLRAEDTAVYYCAARQGQYIYSSM | |
| | RSDSYDYWGQGTLVTVSSGGGGSGGGGSGGGGSGGGGSGGGGSGGGGSEVQLLE | |
| | SGGGLVQPGGSLRLSCAASGFTFRSFGMSWVRQAPGKGPEWVSSISGSGSDTLY | |
| | ADSVKGRFTISRDNSNTLYLQMNSLRPEDTAVYYCTIGGSLSRSSQGTLVTVSS | |
| CRL0278 | EVQLLESGGGLVQPGGSLRLSCAASGFTFRSFGMSWVRQAPGKGPEWVSSISGS | 317 |
| | GSDTLYADSVKGRFTISRDNSKNTLYLQMNSLRPEDTAVYYCTIGGSLSRSSQG | |
| | TLVTVSSGGGGSGGGGSGGGGSEVQLVESGGGLVQPGGSLRLSCAASGRAFSDY | |
| | AMAWFRQAPGQEREFVAGIGWSGGDTLYADSVRGRFTISRDNSKNTLYLQMNSL | |
| | RAEDTAVYYCAARQGQYIYSSMRSDSYDYWGQGTLVTVSS | |
| CRL0279 | EVQLLESGGGLVQPGGSLRLSCAASGFTFRSFGMSWVRQAPGKGPEWVSSISGS | 318 |
| | GSDTLYADSVKGRFTISRDNSKNTLYLQMNSLRPEDTAVYYCTIGGSLSRSSQG | |
| | TLVTVSSGGGGSGGGGSGGGGSGGGGSEVQLVESGGGLVQPGGSLRLSCAASGR | |
| | AFSDYAMAWFRQAPGQEREFVAGIGWSGGDTLYADSVRGRFTISRDNSKNTLYL | |
| | QMNSLRAEDTAVYYCAARQGQYIYSSMRSDSYDYWGQGTLVTVSS | |
| CRL0280 | EVQLLESGGGLVQPGGSLRLSCAASGFTFRSFGMSWVRQAPGKGPEWVSSISGS | 319 |
| | GSDTLYADSVKGRFTISRDNSKNTLYLQMNSLRPEDTAVYYCTIGGSLSRSSQG | |
| | TLVTVSSGGGGSGGGGSGGGGSGGGGSGGGGSEVQLVESGGGLVQPGGSLRLSC | |
| | AASGRAFSDYAMAWFRQAPGQEREFVAGIGWSGGDTLYADSVRGRFTISRDNSK | |
| | NTLYLQMNSLRAEDTAVYYCAARQGQYIYSSMRSDSYDYWGQGTLVTVSS | |
| CRL0281 | EVQLLESGGGLVQPGGSLRLSCAASGFTFRSFGMSWVRQAPGKGPEWVSSISGS | 320 |
| | GSDTLYADSVKGRFTISRDNSKNTLYLQMNSLRPEDTAVYYCTIGGSLSRSSQG | |
| | TLVTVSSGGGGSGGGGSGGGGSGGGGSGGGGSGGGGSEVQLVESGGGLVQPGGS | |
| | LRLSCAASGRAFSDYAMAWFRQAPGQEREFVAGIGWSGGDTLYADSVRGRFTIS | |
| | RDNSKNTLYLQMNSLRAEDTAVYYCAARQGQYIYSSMRSDSYDYWGQGTLVTVS | |
| | S | |

A series of different bi-specific fusion molecules were generated with humanized anti-C5 VHH domains with or without pH-dependent binding. The anti-C5 VHH domains were fused to two different anti-albumin domains to generate bi-specific molecules (shown in Table 9). These constructs were expressed in HEK293F cells and purified using Protein A chromatography. Purified bi-specifics were tested in hemolysis assays and the results are shown in FIGS. 3A-D.

Four bispecific molecules CRL0483, CRL0484, CRL0499 and CRL0500 were prioritized based on binding and functional assays. Biacore affinity measurements for binding to human C5 for CRL0483, CRL0484, CRL0499 and CRL0500 are shown in Table 10 and functional assessments in FIGS. 5, 6 and 7. These four bi-specific molecules were evaluated in in vivo pharmacokinetic studies in cynomolgus monkeys.

### Example 17. Pharmacokinetic Analysis of Bispecific Fusion Molecules

Purified proteins were dosed at 10 mg/kg either intravenously or subcutaneously in cynomolgus monkeys. Three monkeys per dose group per test article were used.

Pharmacokinetics of bispecific molecules was measured by a LC-MS based quantitation assay using signature peptides specific to each construct. The PK profiles are shown in FIGS. 6A and 6B and the parameters are described in Table 20.

TABLE 20
PK parameters after 10 mg/kg of
test articles in cynomolgus monkeys

| | | | | AUC | C_{L} | | |
|---|---|---|---|---|---|---|---|
| | t_{1/2} | tₘₐₓ | Cₘₐₓ | h * | mL/ | V | F |
| Test article | h | h | μg/mL | μg/mL | h/kg | mL/kg | % |
| CRL0483 IV | 139 | 1.33 | 324 | 47900 | 0.211 | 42.0 | |
| CRL0484 IV | 125 | 1 | 382 | 43700 | 0.238 | 43.0 | |
| CRL0483 SC | 103 | 20 | 238 | 46412 | 0.218 | 32.5 | 97 |
| CRL0484 SC | 75.9 | 24 | 161 | 32610 | 0.315 | 34.9 | 75 |
| CRL0499 IV | 170 | 2.11 | 299 | 53773 | 0.184 | 46.9 | |
| CRL0500 IV | 239 | 0.167 | 351 | 51929 | 0.205 | 62.5 | |
| CRL0499 SC | 220 | 32 | 146 | 58666 | 0.173 | 54.2 | 109 |
| CRL0500 SC | 209 | 32 | 161 | 61475 | 0.163 | 49.0 | 118 |

While the disclosure describes various embodiments, it is understood that variations and modifications will occur to those skilled in the art. Therefore, it is intended that the appended claims cover all such equivalent variations. In addition, the section headings used herein are for organizational purposes only and are not to be construed as limiting the subject matter described.

Each embodiment herein described may be combined with any other embodiment or embodiments unless clearly indicated to the contrary. In particular, any feature or embodiment indicated as being preferred or advantageous may be combined with any other feature or features or embodiment or embodiments indicated as being preferred or advantageous, unless clearly indicated to the contrary.

All references cited in this application are expressly incorporated by reference herein.

What is claimed is:

1. A fusion protein comprising an engineered polypeptide that specifically binds to human complement component C5 and an engineered polypeptide that specifically binds to human serum albumin, wherein the engineered polypeptide that specifically binds to human complement component C5 is fused to the polypeptide that specifically binds to human serum albumin either directly or via a peptide linker.

2. The fusion protein of claim 1, wherein the C-terminal residue of the polypeptide that specifically binds to human serum albumin is fused either directly or via a linker to the N-terminal residue of the polypeptide that specifically binds to human complement component C5.

3. The fusion protein of claim 1, wherein the C-terminal residue of the polypeptide that specifically binds to human complement component C5 is fused either directly or via a linker to the N-terminal residue of the polypeptide that specifically binds to human serum albumin.

4. The fusion protein of claim 1, wherein the polypeptide that specifically binds to human complement component C5 comprises an amino acid sequence selected from the group consisting of SEQ ID NOS: 1-12 and fragments thereof; and the polypeptide that specifically binds to human serum albumin comprises an amino acid selected from the group consisting of SEQ ID NOs: 22-34 and fragments thereof.

5. The fusion protein of claim 4, wherein the polypeptide that specifically binds to human complement component C5 comprises the amino acid sequence of SEQ ID NO: 11 and the polypeptide that specifically binds to human serum albumin comprises the amino acid sequence of SEQ ID NO:26.

6. The fusion protein of claim 5, further comprising a peptide linker having an amino acid sequence of SEQ ID NO:102 or 103.

7. The fusion protein of claim 6, wherein the peptide linker comprises the amino acid sequence of SEQ ID NO:102.

8. The fusion protein of claim 1, wherein the fusion protein comprises a sequence that is at least 95% identical to a sequence selected from the group consisting of SEQ ID NOS: 96-101.

9. The fusion protein of claim 8, wherein the fusion protein consists of a sequence selected from the group consisting of SEQ ID NOS: 96-101.

10. The fusion protein of claim 9, wherein the fusion protein consists of a polypeptide sequence of SEQ ID NO:96.

11. The fusion protein of claim 1, wherein the polypeptide that specifically binds to human complement component C5 comprises three complementarity determining regions, CDR1, CDR2 and CDR3, wherein CDR1 comprises any one of the amino acid sequences of SEQ ID NOS: 13-17, CDR2 comprises an amino acid sequences of SEQ ID NO:18 or 19, and CDR3 comprises an amino acid sequences of SEQ ID NO:20 or 21.

12. The fusion protein of claim 1, wherein the polypeptide that specifically binds to human serum albumin comprises three complementarity determining regions, CDR1, CDR2 and CDR3, wherein CDR1 comprises any one of the amino acid sequences of SEQ ID NOS: 35-43, CDR2 comprises any one of the amino acid sequences of SEQ ID NOS: 44-51, and CDR3 comprises any one of the amino acid sequences of SEQ ID NOS: 52-63.

13. The fusion protein of claim 1, wherein either or both of the polypeptides that bind to human complement component C5 or albumin bind in a pH-dependent manner.

14. A pharmaceutical composition comprising a therapeutically effective amount of a fusion protein of any one of claims 1-13 and a pharmaceutically acceptable carrier

15. The pharmaceutical composition of claim 14, further comprising hyaluronidase.

16. An isolated nucleic acid molecule comprising a nucleotide sequence encoding a fusion protein any one of claims 1-13.

17. An expression vector comprising the nucleic acid molecule of claim 16.

18. An isolated host cell comprising the nucleic acid molecule of claim 16.

19. An isolated host cell comprising the expression vector of claim 17.

20. The isolated host cell of claim 19, wherein the host cell is a mammalian cell or a yeast cell.

21. An engineered polypeptide that binds to human complement component C5, wherein the engineered polypeptide comprises an amino acid sequence that is at least 90% identical to a sequence selected from the group consisting of SEQ ID NOS: 1-12.

22. The engineered polypeptide of claim 21, wherein the engineered polypeptide comprises an amino acid sequence selected from the group consisting of SEQ ID NOS: 1-12 and fragments thereof.

23. An engineered polypeptide that specifically binds to human serum albumin, wherein the polypeptide comprises an amino acid sequence that is at least 90% identical to any one of the amino acid sequences of SEQ ID NOS: 22-34.

24. The engineered polypeptide of claim 23, wherein the engineered polypeptide comprises an amino acid sequence selected from the group consisting of SEQ ID NOS: 22-34 and fragments thereof.

25. The engineered polypeptide of claim 24, wherein the polypeptide comprises three complementarity determining regions, CDR1, CDR2 and CDR3, wherein CDR1 comprises an amino acid sequence selected from the group consisting of SEQ ID NOs: 35-43, CDR2 comprises an amino acid sequence selected from the group consisting of SEQ ID NOs: 44-51, and CDR3 comprises an amino acid sequence selected from the group consisting of SEQ ID NOs: 52-63.

26. The engineered polypeptide of claim 22, wherein the polypeptide specifically binds to the same epitope on human serum albumin as Alb1.

27. A method for making a fusion protein of any one of claims 1-13, comprising expressing in a host cell at least one nucleic acid molecule comprising a nucleotide sequence encoding the fusion protein.

28. A therapeutic kit comprising:
(a) a container comprising a label; and
(b). a composition comprising the fusion protein of any one of claims 1-13;
wherein the label indicates that the composition is to be administered to a patient having, or that is suspected of having, a complement-mediated disorder.

29. The kit of claim 28, further comprising hyaluronidase.

30. A method for treating a patient having a complement-mediated disorder, the method comprising administering to the patient a therapeutically effective amount of a fusion protein of any one of claims 1-13.

31. The method of claim 30, wherein the complement-mediated disorder is selected from the group consisting of: rheumatoid arthritis; lupus nephritis; asthma; ischemia-reperfusion injury; atypical hemolytic uremic syndrome; dense deposit disease; paroxysmal nocturnal hemoglobinuria; macular degeneration; hemolysis, elevated liver enzymes, and low platelets (HELLP) syndrome; Guillain-Barre Syndrome; CHAPLE syndrome; myasthenia gravis; neuromyelitis optica; post-hematopoietic stem cell transplant thrombotic microangiopathy (post-HSCT-TMA); post-bone marrow transplant TMA (post-BMT TMA); Degos disease: Gaucher's disease: glomerulonephritis; thrombotic thrombocytopenia purpura (TTP); spontaneous fetal loss; Pauci-immune vasculitis; epidermolysis bullosa; recurrent fetal loss; multiple sclerosis (MS); traumatic brain injury; and injury resulting from myocardial infarction, cardiopulmonary bypass and hemodialysis.
